(19) United States
(12) Patent Application Publication
(10) Pub. No.: US 2025/0161289 A1
(43) Pub. Date: May 22, 2025

(54) KAT6 Targeting Compounds

(71) Applicant: Prelude Therapeutics Incorporated, Wilmington, DE (US)

(72) Inventors: Chun Chen, Claymont, DE (US); Sarah Pawley, Landenberg, PA (US); Andrew Buesking, Wilmington, DE (US); Andrew Combs, Kennett Square, PA (US)

(21) Appl. No.: 18/942,146

(22) Filed: Nov. 8, 2024

Related U.S. Application Data
(60) Provisional application No. 63/597,710, filed on Nov. 10, 2023.

(51) Int. Cl. A61K 31/4545; A61K 31/454; A61P 35/04; C07D 498/04; C07D 519/00

(52) U.S. Cl. CPC A61K 31/4545; A61K 31/454; A61P 35/04; C07D 498/04; C07D 519/00

(57) ABSTRACT

The present disclosure provides bifunctional compounds comprising a target protein binding moiety and a E3 ubiquitin ligase binding moiety, and associated methods of use.

### CROSS REFERENCE TO RELATED APPLICATION

This application which claims benefit under 35 U.S.C. § 119(e) of Provisional U.S. Patent Application No. 63/597,710, filed Nov. 10, 2023, the contents of which are hereby incorporated by reference in its entirety.

### TECHNICAL FIELD

The description provides bifunctional compounds comprising a target protein binding moiety and a E3 ubiquitin ligase binding moiety, and associated methods of use. The bifunctional compounds are useful as modulators of targeted KAT6 proteins, which are degraded and/or otherwise inhibited by bifunctional compounds according to the present disclosure.

### BACKGROUND OF THE INVENTION

Epigenetic control of transcription via chromatin remodeling is a substantial regulatory mechanism that ultimately impacts the functional expression of many proteins. Aberrations to epigenetic regulators are contributors to human diseases and are considered major oncogenic drivers in many cancers. Regulation of histones play an essential role in this process and epigenetic regulators modify histone proteins through assorted post-translational modifications such as acetylation and methylation to alter, for example, chromatin accessibility. Histone acetylation is catalyzed by histone acetyltransferases (HATs), and dysregulation of this class of epigenetic regulators is linked to transcriptional alterations and cancer (1, 2).

The MYST family of acetyltransferases have been linked to several types of cancer and contain a conserved catalytic domain (MYST domain). Lysine acetyltransferase 6a (KAT6A, MOZ, MYST3) is a member of this family, promotes histone acetylation, and is paralogous to lysine acetyltransferase 6b (KAT6B) (3, 4). KAT6A reportedly acetylates lysine 9 (H3K9ac), lysine 14 (H3K14ac)(4), and lysine 23 (H3K23ac) (5) of histone H3, although the functional dependency on these sites, and other potential substrates, remains poorly understood. In addition to histone proteins, KAT6A reportedly acetylates non-histone proteins such as p53 (6), but its role in regulating non-histone substrates is not clear. KAT6A can form a protein complex with inhibitor of growth family member 5 (ING5), bromodomain and PHD finger containing 1, 2 or 3 (BRPF1, BRPF2, BRPF3), and myst/Esa1 associated factor 6 (EAF6), which may enhance its gene regulatory capacity (4, 7). Mechanistically, KAT6A is linked to regulation of cellular processes such as senescence, cell cycle progression, and hematopoietic stem cell maintenance (4, 7, 8).

More recently, KAT6A was shown to epigenetically regulate estrogen receptor alpha (ER alpha) expression via its HAT domain in KAT6A amplified breast cancer cell lines. Loss of KAT6A was associated with reduced proliferation in vitro and tumor growth in vivo and its overexpression correlates with worse clinical outcome in estrogen receptor positive (ER+) breast cancers (9). Inhibitors of KAT6A reportedly show greater sensitivity in the ER+ luminal subtype (ER/PR+/HER2−) (10). KAT6A is suggested to be an oncogenic in breast, brain, hematological, gynecological, and other cancers (5, 9, 11, 12).

KAT6A is frequently altered in many types of cancers, most often by copy number amplification or recurrent chromosomal translocations. Located within the recurrently amplified chromosomal 8p11-12 amplicon, KAT6A amplification is observed in numerous cancers including breast, lung, prostate, blood, bladder, uterine, endometrial, ovarian, esophageal, head and neck, stomach, colon, and other cancers (9, 13, 14). Especially in hematological cancers, recurrent rearrangements lead to several fusion proteins like KAT6A-CBP, KAT6A-p300, KAT6A-TIF2, KAT6A-NcoA3, and KAT6A-LEUTX (7). Recently, KAT6A has been implicated as important for the growth of MLL-rearranged AML (11).

To date, only one KAT6A targeted small molecule inhibitor is currently in clinical trials (PF-07248144, Phase 1, Pfizer) to study effects in locally advanced or metastatic ER+/HER2− breast, castration-resistant prostate, and non-small cell lung cancer as a single agent or in combination with either fulvestrant or letrozole+palbociclib (NCT04606446) (15). It remains unclear whether traditional small molecule inhibitors of KAT6A can unlock the full potential of targeting these or other cancers in a selective manner. Therefore, exploring a targeted protein degradation (TPD) approach to develop potent and selective KAT6A degraders could enhance the therapeutic window in cancers dependent on KAT6A for growth, proliferation, or survival.

In light of the established role of KATs in diseases such as cancer, a need exists for new modulators of these proteins.

### REFERENCES

1. Nair S S, Kumar R. Chromatin remodeling in cancer: a gateway to regulate gene transcription. Mol Oncol. 2012; 6(6):611-9.
2. Farria A, Li W, Dent S Y. KATs in cancer: functions and therapies. Oncogene. 2015; 34(38):4901-13.
3. Avvakumov N, Côté J. The MYST family of histone acetyltransferases and their intimate links to cancer. Oncogene. 2007; 26(37):5395-407.
4. Su J, Wang X, Bai Y, Sun M, Yao Y, Duan Y. The role of MOZ/KAT6A in hematological malignancies and advances in MOZ/KAT6A inhibitors. Pharmacol Res. 2021; 174:105930.
5. Lv D, Jia F, Hou Y, Sang Y, Alvarez A A, Zhang W, et al. Histone Acetyltransferase KAT6A Upregulates PI3K/AKT Signaling through TRIM24 Binding. Cancer Res. 2017; 77(22):6190-201.
6. Rokudai S, Laptenko O, Arnal S M, Taya Y, Kitabayashi I, Prives C. MOZ increases p53 acetylation and premature senescence through its complex formation with PML. Proc Natl Acad Sci USA. 2013; 110(10):3895-900.
7. Huang F, Abmayr S M, Workman J L. Regulation of KAT6 Acetyltransferases and Their Roles in Cell Cycle Progression, Stem Cell Maintenance, and Human Disease. Mol Cell Biol. 2016; 36(14):1900-7.
8. Sheikh B N, Phipson B, El-Saafin F, Vanyai H K, Downer N L, Bird M J, et al. MOZ (MYST3, KAT6A) inhibits senescence via the INK4A-ARF pathway. Oncogene. 2015; 34(47):5807-20.
9. Yu L, Liang Y, Cao X, Wang X, Gao H, Lin S Y, et al. Identification of MYST3 as a novel epigenetic activator of ERα frequently amplified in breast cancer. Oncogene. 2017; 36(20):2910-8.
10. Sharma S, Chung J, Uryu S, Rickard A, Nady N, Khan S, et al. Abstract 1130: First-in-class KAT6A/KAT6B inhibitor CTx-648 (PF-9363) demonstrates potent anti-tumor activity in ER+ breast cancer with KAT6A dysregulation. Cancer Research. 2021; 81(13_Supplement):1130-.
11. Yan F, Li J, Milosevic J, Petroni R, Liu S, Shi Z, et al. KAT6A and ENL Form an Epigenetic Transcriptional Control Module to Drive Critical Leukemogenic Gene-Expression Programs. Cancer Discov. 2022; 12(3):792-811.
12. Liu W, Zhan Z, Zhang M, Sun B, Shi Q, Luo F, et al. KAT6A, a novel regulator of β-catenin, promotes tumorigenicity and chemoresistance in ovarian cancer by acetylating COP1. Theranostics. 2021; 11(13):6278-92.
13. Saglam O, Tang Z, Tang G, Medeiros L J, Toruner G A. KAT6A amplifications are associated with shorter progression-free survival and overall survival in patients with endometrial serous carcinoma. PLoS One. 2020; 15(9):e0238477.
14. Cerami E, Gao J, Dogrusoz U, Gross B E, Sumer S O, Aksoy B A, et al. The cBio cancer genomics portal: an open platform for exploring multidimensional cancer genomics data. Cancer Discov. 2012; 2(5):401-4.
15. ClinicalTrials.gov. NCT04606446: Study of PF-07248144 in Advanced or Metastatic Solid Tumors (KAT6) Bethesda (MD): National Library of Medicine (US); 2020 [Available from: https://clinicaltrials.gov/ct2/show/NCT04606446.

### SUMMARY OF THE INVENTION

The present disclosure is directed to compounds of Formula (I):

ULM-Y-PTM (I)
or a pharmaceutically acceptable salt or solvate thereof,
wherein PTM is a moiety of Formula IA:

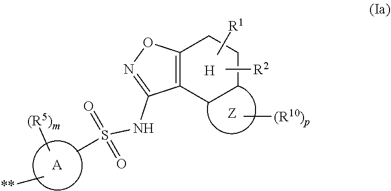

wherein
   ring H is a 6-membered aromatic ring or a 6-membered heteroaromatic ring;
   ring Z is a 5- to 7-membered cycloalkenyl ring, a 5- to 7-membered heterocycloalkenyl ring, or 5- to 7-membered heteroaromatic ring;
   Y is a covalent bond, or chemical moiety that links PTM to ULM;
   ** is a point of attachment to Y;
   Ring A is a C₆-C₁₀ aryl ring or a 5-10 membered heteroaryl ring;
   R¹ is H or 5-6 membered heteroaryl ring optionally substituted by C₁-C₃ alkyl;
   R² is H or —(C(R′)₂)ₙ-(5-9 membered heteroaryl ring) optionally substituted by halogen, C₁-C₃ alkyl, —CH₂OH, or —OH;
   each R⁵ is independently D, halogen, oxo, —OH, —CN, —NO₂, —C₁-C₆alkyl, —C₂-C₆alkenyl, —C₂-C₆alkynyl, haloalkyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(=NR^{b})R^{b}, —SF₅, —P(O)R^{b}R^{b}, —P(O)(OR^{b})(OR^{b}), —B(OR^{d})(OR^{c}) or —S(O)₂R^{b};
   m is 0, 1, 2, 3, or 4;
   each R⁸ is independently H, halogen, or C₁-C₄ alkyl;
   n is 0 or 1;
   each R¹⁰ is independently H, halogen, C₁₋₄alk-O—C₁₋₄alkyl, C₁-C₄ alkoxy or C₁-C₄ alkyl;
   p is 1, 2, 3, 4, 5, 6, 7 or 8;
   each R^{a} is independently H, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)R^{c}R^{b}, —P(O)OR^{c}OR^{b}, —S(O)R^{b}, —S(O)NR^{c}R^{d}, —S(O)₂R^{b}, —S(O)₂NR^{c}R^{d}, SiR^{b}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl;
   each R^{b}, is independently H, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl;
   each R^{c} or R^{d} is independently H, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; or
   R^{c} and R^{d}, together with the atom to which they are both attached, form a monocyclic or multicyclic heterocycloalkyl, or a monocyclic or multicyclic heterocyclo-alkenyl group; and
   ULM is a small molecule E3 Ubiquitin Ligase binding moiety that binds a Cereblon E3 Ubiquitin Ligase.

Stereoisomers of the compounds of Formula I, and the pharmaceutical salts and stereoisomers thereof, are also contemplated, described, and encompassed herein. Methods of using compounds of Formula I are described, as well as pharmaceutical compositions including the compounds of Formula I.

### DETAILED DESCRIPTION OF ILLUSTRATIVE EMBODIMENTS

Unless otherwise defined, all technical and scientific terms used herein have the same meaning as commonly understood by one of ordinary skill in the art to which this disclosure pertains. The terminology used in the description is for describing particular embodiments only and is not intended to be limiting of the disclosure.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either both of those included limits are also included in the disclosure.

The following terms are used to describe the present disclosure. In instances where a term is not specifically defined herein, that term is given an art-recognized meaning by those of ordinary skill applying that term in context to its use in describing the present disclosure.

The articles “a” and “an” as used herein and in the appended claims are used herein to refer to one or to more than one (e.g., to at least one) of the grammatical object of the article unless the context clearly indicates otherwise. By way of example, “an element” means one element or more than one element.

The terms “co-administration” and “co-administering” or “combination therapy” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain preferred aspects, one or more of the present compounds described herein, are co-administered in combination with at least one additional bioactive agent, especially including an anticancer agent. In particularly preferred aspects, the co-administration of compounds results in synergistic activity and/or therapy, including anticancer activity.

The term “compound”, as used herein, unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives, including prodrug and/or deuterated forms thereof where applicable, in context. Deuterated small molecules contemplated are those in which one or more of the hydrogen atoms contained in the drug molecule have been replaced by deuterium.

Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. It is noted that in describing the present compounds, numerous substituents and variables associated with same, among others, are described. It is understood by those of ordinary skill that molecules which are described herein are stable compounds as generally described hereunder.

The term “ubiquitin ligase” refers to a family of proteins that facilitate the transfer of ubiquitin to a specific substrate protein, targeting the substrate protein for degradation. For example, an E3 ubiquitin ligase protein that alone or in combination with an E2 ubiquitin-conjugating enzyme causes the attachment of ubiquitin to a lysine on a target protein, and subsequently targets the specific protein substrates for degradation by the proteasome. Thus, E3 ubiquitin ligase alone or in complex with an E2 ubiquitin conjugating enzyme is responsible for the transfer of ubiquitin to targeted proteins. In general, the ubiquitin ligase is involved in polyubiquitination such that a second ubiquitin is attached to the first; a third is attached to the second, and so forth. Polyubiquitination marks proteins for degradation by the proteasome. However, there are some ubiquitination events that are limited to mono-ubiquitination, in which only a single ubiquitin is added by the ubiquitin ligase to a substrate molecule. Mono-ubiquitinated proteins are not targeted to the proteasome for degradation but may instead be altered in their cellular location or function, for example, via binding other proteins that have domains capable of binding ubiquitin. Further complicating matters, different lysines on ubiquitin can be targeted by an E3 to make chains. The most common lysine is Lys48 on the ubiquitin chain. This is the lysine used to make polyubiquitin, which is recognized by the proteasome.

As used herein, the term “alkyl”, by itself or as part of another substituent, means, unless otherwise stated, a straight or branched chain hydrocarbon radical having up to twelve carbon atoms. In some embodiments, the number of carbon atoms is designated (i.e., C₁-C₈ means one to eight carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. Alkyl groups may be optionally substituted as provided herein. In some embodiments, the alkyl group is a C₁-C₆ alkyl; in some embodiments, it is a C₁-C₄ alkyl.

When a range of carbon atoms is used herein, for example, C₁-C₆, all ranges, as well as individual numbers of carbon atoms are encompassed. For example, “C₁-C₃” includes C₁-C₃, C₁-C₂, C₂-C₃, C₁, C₂, and C₃.

The term “optionally substituted”, as used in combination with a substituent defined herein, means that the substituent may, but is not required to be, substituted with one or more suitable functional groups or other substituents as provided herein. For example, a substituent may be optionally substituted with one or more of: halo, cyano, C₁₋₆ alkyl, C₃₋₆ cycloalkyl, C₂₋₆ alkenyl, C₂₋₆ alkynyl, halo(C₁₋₆)alkyl, C₁₋₆ alkoxy, halo(C₁₋₆ alkoxy), C₁₋₆ alkylthio, C₁₋₆ alkylamino, NH₂, NH(C₁₋₆ alkyl), N(C₁₋₆ alkyl)₂, NH(C₁₋₆alkoxy), N(C₁₋₆ alkoxy)₂, C(O)NHC₁₋₆ alkyl, C(O)N(C₁₋₆ alkyl)₂, C(O)NH₂, C(O)C₁₋₆ alkyl, C(O)₂C₁₋₆ alkyl, —NHCO(C₁₋₆ alkyl), N(C₁₋₆ alkyl)CO(C₁₋₆ alkyl), S(O)C₁₋₆ alkyl, S(O)₂C₁₋₆ alkyl, oxo, 6-12 membered aryl, benzyl, pyridinyl, pyrazolyl, thiazolyl, isothiazolyl, or other 5 to 12 membered heteroaryl groups. In some embodiments, each of the above optional substituents are themselves optionally substituted by one or two groups.

The term “cycloalkyl” as used herein refers to a 3-12 membered cyclic alkyl group, and includes bridged (e.g., adamantine) and spirocycles (e.g., spiro[3.5]nonane). Cycloalkyl groups may be fully saturated or partially unsaturated. The term “cycloalkyl” also includes multiple condensed ring systems (e.g., ring systems comprising 2, 3 or 4 rings) wherein a single cycloalkyl ring (as defined above) can be condensed with one or more groups selected from heterocycles, carbocycles, aryls, or heteroaryls to form the multiple condensed ring system. Such multiple condensed ring systems may be optionally substituted with one or more (e.g., 1, 2, 3 or 4) oxo groups on the carbocycle or heterocycle portions of the multiple condensed ring. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a cycloalkyl) can be at any position of the cycloalkylic ring. Examples of cycloalkyl groups include cyclopropyl, cyclobutyl, cyclopentyl, cycloheptyl, cyclohexyl, cycloheptyl, cyclooctyl, indenyl, bicyclo[2.2.1]heptanyl, bicyclo[3.1.1]heptanyl, bicyclo[4.1.0]heptanyl, spiro[3.3]heptanyl, and spiro[3.4]octanyl. In some embodiments, the cycloalkyl group is a 3-7 membered cycloalkyl.

The term “alkenyl” as used herein refers to C₂-C₁₂ alkyl group that contains at least one carbon-carbon double bond. In some embodiments, the alkenyl group is optionally substituted. In some embodiments, the alkenyl group is a C₂-C₆ alkenyl.

The term “cycloalkenyl” when used alone or as part of a substituent group refers to monocyclic or multicyclic, partially saturated ring structure having from 3 to 10 carbon atoms (“C₃-C₁₀”), preferably from 3 to 6 carbon atoms (“C₃-C₆”). Cycloalkenyl groups of the disclosure include monocyclic groups, as well as multicyclic groups such as bicyclic and tricyclic groups. In those embodiments having at least one multicyclic cycloalkenyl group, the cyclic groups can share one common atom (i.e., spirocyclic). In other embodiments having at least one multicyclic cycloalkenyl group, the cyclic groups share two common atoms (e.g., fused or bridged). The term —C₃-C₆ cycloalkenyl refers to a cycloalkenyl group having between three and six carbon atoms. The cycloalkenyl group may be attached at any carbon atom of the partially saturated ring such that the result is a stable structure. Cycloalkenyl groups include groups in which the partially saturated ring is fused to an aryl group. Examples of cycloalkenyl groups include, for example, cyclopropenyl (C₃), cyclobutenyl (C₄), cyclopropenylmethyl (C₄), cyclopentenyl (C₅), cyclohexenyl (C₆), 1-methylcyclopropenyl (C₄), 2-methylcyclopentenyl (C₄), adamantenyl (C₁₀), spiro[3.3]heptenyl, bicyclo[3.3.0]octenyl, indanyl, and the like. Cycloalkenyl groups of the disclosure are optionally substituted. Unless otherwise specified, in those embodiments wherein the cycloalkenyl group is substituted, the cycloalkenyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the cycloalkenyl group is optionally substituted by 1-6 R groups selected from H, D, halogen, —OH, —CN, —OR^{a}, —SR^{a}, —NR^{a}R^{d}, or NR^{c}R^{d}.

The term “alkynyl” as used herein refers to C₂-C₁₂ alkyl group that contains at least one carbon-carbon triple bond. In some embodiments, the alkenyl group is optionally substituted. In some embodiments, the alkynyl group is a C₂-C₆ alkynyl.

The terms “alkoxy,” “alkylamino” and “alkylthio”, are used in their conventional sense, and refer to those alkyl groups attached to the remainder of the molecule via an oxygen atom (“oxy”), an amino group (“amino”) or thio group. The term “alkylamino” includes mono-di-alkylamino groups, the alkyl portions can be the same or different.

The terms “halo” or “halogen”, by itself or as part of another substituent, means a fluorine, chlorine, bromine, or iodine atom.

The term “heteroalkyl” refers to an alkyl group in which one or more carbon atom has been replaced by a heteroatom selected from S, O, P and N. Exemplary heteroalkyls include alkyl ethers, secondary and tertiary alkyl amines, alkyl amides, alkyl sulfides, and the like. The group may be a terminal group or a bridging group. As used herein reference to the normal chain when used in the context of a bridging group refers to the direct chain of atoms linking the two terminal positions of the bridging group.

The term “aryl” as used herein refers to a single, all carbon aromatic ring or a multiple condensed all carbon ring system wherein at least one of the rings is aromatic. For example, in certain embodiments, an aryl group has 6 to 12 carbon atoms. Aryl includes a phenyl radical. Aryl also includes multiple condensed ring systems (e.g., ring systems comprising 2, 3 or 4 rings) having about 9 to 12 carbon atoms in which at least one ring is aromatic and wherein the other rings may be aromatic or not aromatic. Such multiple condensed ring systems are optionally substituted with one or more (e.g., 1, 2 or 3) oxo groups on any carbocycle portion of the multiple condensed ring system. The rings of the multiple condensed ring system can be connected to each other via fused, spiro and bridged bonds when allowed by valency requirements. It is to be understood that the point of attachment of a multiple condensed ring system, as defined above, can be at any position of the aromatic ring. Non-limiting examples of aryl groups include, but are not limited to, phenyl, indenyl, naphthyl, 1, 2, 3, 4-tetrahydro-naphthyl, and the like.

The term “heteroaryl” as used herein refers to a single aromatic ring that has at least one atom other than carbon in the ring, wherein the atoms are selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from heteroaryls (to form for example a naphthyridinyl such as 1,8-naphthyridinyl), heterocycles, (to form for example a 1, 2, 3, 4-tetrahydronaphthyridinyl such as 1,2,3,4-tetrahydro-1,8-naphthyridinyl), carbocycles (to form for example 5,6,7,8-tetrahydroquinolyl) and aryls (to form for example indazolyl) to form the multiple condensed ring system. Thus, a heteroaryl (a single aromatic ring or multiple condensed ring system) has about 1-20 carbon atoms and about 1-6 heteroatoms within the heteroaryl ring. A heteroaryl (a single aromatic ring or multiple condensed ring system) can also have about 5 to 12 or about 5 to 10 members within the heteroaryl ring. Multiple condensed ring systems may be optionally substituted with one or more (e.g., 1, 2, 3 or 4) oxo groups on the carbocycle or heterocycle portions of the condensed ring. The rings of a multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heteroaryl) can be at any position of the heteroaryl ring. It is also to be understood that the point of attachment for a heteroaryl or heteroaryl multiple condensed ring system can be at any suitable atom of the heteroaryl ring including a carbon atom and a heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl benzofuranyl, benzimidazolyl, thianaphthenyl, pyrrolo[2,3-b]pyridinyl, quinazolinyl-4(3H)-one, triazolyl, 4,5,6,7-tetrahydro-1H-indazole and 3b,4,4a,5-tetrahydro-1H-cyclopropa[3,4]cyclo-penta[1,2-c]pyrazole. In one embodiment the term “heteroaryl” refers to a single aromatic ring containing at least one heteroatom. For example, the term includes 5-membered and 6-membered monocyclic aromatic rings that include one or more heteroatoms. Non-limiting examples of heteroaryl include but are not limited to pyridyl, furyl, thiazole, pyrimidine, oxazole, and thiadiazole.

The term “heterocyclyl” or “heterocycle” as used herein refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The ring may be substituted with one or more (e.g., 1, 2 or 3) oxo groups and the sulfur and nitrogen atoms may also be present in their oxidized forms. Exemplary heterocycles include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more groups selected from heterocycles (to form for example a 1,8-decahydronapthyridinyl), carbocycles (to form for example a decahydroquinolyl) and aryls to form the multiple condensed ring system. Thus, a heterocycle (a single saturated or single partially unsaturated ring or multiple condensed ring system) has about 2-20 carbon atoms and 1-6 heteroatoms within the heterocycle ring. Such multiple condensed ring systems may be optionally substituted with one or more (e.g., 1, 2, 3 or 4) oxo groups on the carbocycle or heterocycle portions of the multiple condensed ring. The rings of the multiple condensed ring system can be connected to each other via fused, spiro and bridged bonds when allowed by valency requirements. It is to be understood that the individual rings of the multiple condensed ring system may be connected in any order relative to one another. Accordingly, a heterocycle (a single saturated or single partially unsaturated ring or multiple condensed ring system) has about 3-20 atoms including about 1-6 heteroatoms within the heterocycle ring system. It is also to be understood that the point of attachment of a multiple condensed ring system (as defined above for a heterocyclyl) can be at any position of the heterocyclic ring. It is also to be understood that the point of attachment for a heterocycle or heterocycle multiple condensed ring system can be at any suitable atom of the heterocyclic ring including a carbon atom and a heteroatom (e.g., a nitrogen). In one embodiment the term heterocycle includes a C₂₋₂₀ heterocycle. In one embodiment the term heterocycle includes a C₂₋₇ heterocycle. In one embodiment the term heterocycle includes a C₂₋₅ heterocycle. In one embodiment the term heterocycle includes a C₂₋₄ heterocycle. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one N-methylpiperidine, imidazolidine, pyrazolidine, butyrolactam, valerolactam, imidazolidinone, hydantoin, dioxolane, phthalimide, 1,4-dioxane, thiomorpholine, thiomorpholine-S-oxide, thiomorpholine-S,S-oxide, pyran, 3-pyrroline, thiopyran, pyrone, tetrhydrothiophene, quinuclidine, tropane, 2-azaspiro[3.3]heptane, 2,7-diazaspiro[3.5]nonane, (1R,5S)-3-azabicyclo[3.2.1]octane, (1s,4s)-2-azabicyclo[2.2.2]octane, (1R,4R)-2-oxa-5-azabicyclo[2.2.2]octane and pyrrolidin-2-one. In one embodiment the term “heterocycle” refers to a monocyclic, saturated or partially unsaturated, 3-8 membered ring having at least one heteroatom. For example, the term includes a monocyclic, saturated or partially unsaturated, 4, 5, 6, or 7 membered ring having at least one heteroatom. Non-limiting examples of heterocycle include aziridine, azetidine, pyrrolidine, piperidine, piperidine, piperazine, oxirane, morpholine, and thiomorpholine. The term “9- or 10-membered heterobicycle” as used herein refers to a partially unsaturated or aromatic fused bicyclic ring system having at least one heteroatom. For example, the term 9- or 10-membered heterobicycle includes a bicyclic ring system having a benzo ring fused to a 5-membered or 6-membered saturated, partially unsaturated, or aromatic ring that contains one or more heteroatoms.

The term “heterocycloalkyl” when used alone or as part of a substituent group refers to any three to twelve membered monocyclic or multicyclic, saturated ring structure containing at least one heteroatom selected from the group consisting of O, N and S. Heterocycloalkyl groups of the disclosure include monocyclic groups, as well as multicyclic groups such as bicyclic and tricyclic groups. In those embodiments having at least one multicyclic heterocycloalkyl group, the cyclic groups can share one common atom (i.e., spirocyclic). In other embodiments having at least one multicyclic heterocycloalkyl group, the cyclic groups share two common atoms (e.g., fused or bridged). The term —C₃-C₆ heterocycloalkyl refers to a heterocycloalkyl group having between three and six carbon ring atoms. The heterocycloalkyl group may be attached at any heteroatom or carbon atom of the group such that the result is a stable structure. Examples of heterocycloalkyl groups include, but are not limited to, azepanyl, aziridinyl, azetidinyl, pyrrolidinyl, dioxolanyl, imidazolidinyl, pyrazolidinyl, piperazinyl, piperidinyl, dioxanyl, morpholinyl, dithianyl, thiomorpholinyl, oxazepanyl, oxiranyl, oxetanyl, quinuclidinyl, tetrahydrofuranyl, tetrahydropyranyl, piperazinyl, azepanyl, diazepanyl, oxepanyl, dioxepanyl, azocanyl diazocanyl, oxocanyl, dioxocanyl, azaspiro[2.2]pentanyl, oxaazaspiro[3.3]heptanyl, oxaspiro[3.3]heptanyl, dioxaspiro[3.3]heptanyl, 3-azabicyclo[3.1.0]hexanyl,

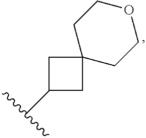 [SMILES: *C1CC2(CCOCC2)C1]

and the like. Heteroycloalkyl groups of the disclosure are optionally substituted. Unless otherwise specified, in those embodiments wherein the heterocycloalkyl group is substituted, the heterocycloalkyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the heterocycloalkyl group is optionally substituted by 1-6 R groups selected from H, D, halogen, —OH, —CN, —OR^{a}, —SR^{a}, —NR^{a}R^{d}, or NR^{c}R^{d}.

The term “heterocycloalkenyl” when used alone or as part of a substituent group refers to any three to twelve membered monocyclic or multicyclic, partially saturated ring structure containing at least one heteroatom selected from the group consisting of O, N and S. Heterocycloalkenyl groups of the disclosure include monocyclic groups, as well as multicyclic groups such as bicyclic and tricyclic groups. In those embodiments having at least one multicyclic heterocycloalkyenyl group, the cyclic groups can share one common atom (i.e., spirocyclic). In other embodiments having at least one multicyclic heterocycloalkenyl group, the cyclic groups share two common atoms (e.g., fused or bridged). The term —C₃-C₆ heterocycloalkenyl refers to a heterocycloalkenyl group having between three and six carbon atoms. The heterocycloalkenyl group may be attached at any heteroatom or carbon atom of the partially saturated ring such that the result is a stable structure. Heterocycloalkenyl groups include groups in which the partially saturated ring is fused to an aryl group, such as, for example isoindoline,

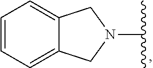 [SMILES: *N1Cc2ccccc2C1]

or in which the partially saturated ring is fused to a heteroaryl group, such as, for example, 6,7-dihydro-5H-pyrrolo[3,4-b]pyridine,

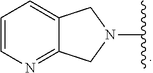 [SMILES: *N1Cc2cccnc2C1]

Heteroycloalkenyl groups of the disclosure are optionally substituted. Unless otherwise specified, in those embodiments wherein the heterocycloalkenyl group is substituted, the heterocycloalkenyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the heterocycloalkenyl group is optionally substituted by 1-6 R groups selected from H, D, halogen, —OH, —CN, —OR^{a}, —SR^{a}, —NR^{a}R^{d}, or NR^{c}R^{d}.

As used herein, the term “heteroatom” is meant to include oxygen (O), nitrogen (N), sulfur (S) and silicon (Si). The nitrogen and sulfur can be in an oxidized form when feasible.

As used herein, the term “chiral” refers to molecules which have the property of non-superimposability of the mirror image partner, while the term “achiral” refers to molecules which are superimposable on their mirror image partner.

As used herein, the term “stereoisomers” refers to compounds which have identical chemical constitution but differ with regard to the arrangement of the atoms or groups in space, e.g., enantiomers, diastereomers, tautomers.

The term “patient” or “subject” is used throughout the specification to describe an animal, preferably a human or a domesticated animal, to whom treatment, including prophylactic treatment, with the compositions according to the present disclosure is provided. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

The term “effective” is used to describe an amount of a compound, composition or component which, when used within the context of its intended use, effects an intended result. The term effective subsumes all other effective amount or effective concentration terms, which are otherwise described or used in the present application.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, e.g., in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

A “solvate” refers to a physical association of a compound of Formula I with one or more solvent molecules.

“Treating” or “treatment” of any disease or disorder refers, in one embodiment, to ameliorating the disease or disorder (e.g., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

In one embodiment, the disclosure is directed to a compound of Formula (I):

ULM-Y-PTM (I)
or a pharmaceutically acceptable salt or solvate thereof,
wherein PTM is a moiety of Formula IA:

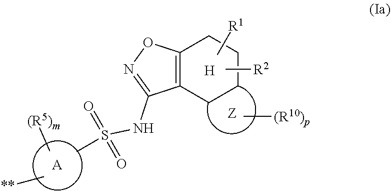

wherein
   ring H is a 6-membered aromatic ring or a 6-membered heteroaromatic ring;
   ring Z is a 5- to 7-membered cycloalkenyl ring, a 5- to 7-membered heterocycloalkenyl ring, or 5- to 7-membered heteroaromatic ring;
   Y is a covalent bond, or chemical moiety that links PTM to ULM;
   ** is a point of attachment to Y;
   Ring A is a C₆-C₁₀ aryl ring or a 5-10 membered heteroaryl ring;
   R¹ is H or 5-6 membered heteroaryl ring optionally substituted by C₁-C₃ alkyl;
   R² is H or —(C(R⁸)₂)ₙ-(5-9 membered heteroaryl ring) optionally substituted by halogen, C₁-C₃ alkyl, —CH₂OH, or —OH;
   each R⁵ is independently D, halogen, oxo, —OH, —CN, —NO₂, —C₁-C₆alkyl, —C₂-C₆alkenyl, —C₂-C₆alkynyl, haloalkyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(=NR^{b})R^{b}, —SF₅, —P(O)R^{b}R^{b}, —P(O)(OR^{b})(OR^{b}), —B(OR^{d})(OR^{c}) or —S(O)₂R^{b};
   m is 0, 1, 2, 3, or 4;
   each R⁸ is independently H, halogen, or C₁-C₄ alkyl;
   n is 0 or 1;
   each R¹⁰ is independently H, halogen, C₁₋₄alk-O—C₁₋₄alkyl, C₁-C₄ alkoxy or C₁-C₄ alkyl;
   p is 1, 2, 3, 4, 5, 6, 7 or 8;
   each R^{a} is independently H, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)R^{c}R^{b}, —P(O)OR^{c}OR^{b}, —S(O)R^{b}, —S(O)NR^{c}R^{d}, —S(O)₂R^{b}, —S(O)₂NR^{c}R^{d}, SiR^{b}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl;
   each R^{b}, is independently H, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl;
   each R^{c} or R^{d} is independently H, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; or
   R^{c} and R^{d}, together with the atom to which they are both attached, form a monocyclic or multicyclic heterocycloalkyl, or a monocyclic or multicyclic heterocyclo-alkenyl group; and
   ULM is a small molecule E3 Ubiquitin Ligase binding moiety that binds a Cereblon E3 Ubiquitin Ligase.

According to the disclosure, Ring H in Formula IA is a 6-membered aromatic ring or a 6-membered heteroaromatic ring. In some embodiments, Ring H in Formula IA is 6-membered aromatic ring. In some embodiments, Ring H in Formula IA is a phenyl group. In other embodiments, Ring H in Formula IA is a 6-membered heteroaryl group.

According to the disclosure, Ring Z in Formula IA is ring Z is a 5- to 7-membered cycloalkenyl ring, a 5- to 7-membered heterocycloalkenyl ring, or a 5- to 7-membered heteroaromatic ring. It will be understood by those of skill in the art, that because ring Z is fused to ring H, and ring H is aromatic or heteroaromatic, the bond that is shared by ring Z and ring H is, in essence, a double bond. Ring Z, therefore, is either a cycloalkenyl or heterocyclo-alkenyl ring.

In some embodiments, Ring Z in Formula IA is a 5- to 7-membered cycloalkenyl ring. In some embodiments, Ring Z in Formula IA is a cyclopentenyl ring. In other embodiments, Ring Z in Formula IA is a cyclohexenyl ring.

In some embodiments, Ring Z in Formula IA is a 5- to 7-membered heterocyclo-alkenyl ring. In some embodiments, Ring Z in Formula IA is a dihydrofuran ring, a dihydro-2H-pyran ring, a dihydro-dioxepine ring, a dioxole ring, or a dihydrothiophene ring.

In some embodiments, Ring Z in Formula IA is a dihydrofuran ring. In some embodiments, Ring Z in Formula IA is a dihydro-2H-pyran ring. In other embodiments, Ring Z in Formula IA is a dihydro-dioxepine ring. In other embodiments, Ring Z in Formula IA is a dioxole ring. In other embodiments, Ring Z in Formula IA is a dihydrothiophene ring.

According to the disclosure, Ring Z in Formula IA is a 5- to 7-membered hetero-aromatic ring. In some embodiments, Ring Z in Formula IA is a furan ring or an imidazole ring. In some embodiments, Ring Z in Formula IA is a furan ring. In other embodiments, Ring Z in Formula IA is an imidazole ring.

According to the disclosure, Ring A in Formula IA is a C₆-C₁₀ membered aryl group or a 5-10 membered heteroaryl group. In some embodiments, Ring A in Formula IA is a C₆-C₁₀ membered aryl group. In some embodiments, Ring A in Formula IA is a phenyl group. In other embodiments, Ring A in Formula IA is a 5-10 membered heteroaryl group.

According to the disclosure, R¹ in Formula IA is H, D, or 5-6 membered heteroaryl optionally substituted by methyl. In some embodiments, R¹ in Formula IA is H. In some embodiments, R¹ in Formula IA is D. In other embodiments, R¹ in Formula IA is 5-6 membered heteroaryl optionally substituted by methyl.

According to the disclosure, R² in Formula IA is H, D, or —(C(R⁸)₂)ₙ-(5-9 membered heteroaryl) optionally substituted by halogen, C₁-C₃ alkyl, —CH₂OH, or —OH. In some embodiments, R² in Formula IA is H. In some embodiments, R² in Formula IA is D. In other embodiments, R² in Formula IA is —(C(R⁸)₂)ₙ-(5-9 membered heteroaryl) optionally substituted by halogen, C₁-C₃ alkyl, —CH₂OH, or —OH.

In yet other embodiments, R² in Formula IA is —(C(R′)₂)ₙ-(5-6 membered heteroaryl) optionally substituted by halogen, C₁-C₃ alkyl, —CH₂OH, or —OH. In some embodiments, when R² in Formula IA is —(C(R′)₂)ₙ-(5-6 membered heteroaryl), the 5-6 membered heteroaryl is a pyrazole, a pyrrole, a pyridine or a pyridazine. In some embodiments, when R² in Formula IA is —(C(R′)₂)ₙ-(5-6 membered heteroaryl), the 5-6 membered heteroaryl is a pyrazole.

According to the disclosure, R⁸ in Formula IA is H, D, halogen, or C₁-C₄ alkyl. In some embodiments, R⁸ in Formula IA is H. In some embodiments, R⁸ in Formula IA is D. In other embodiments, R⁸ in Formula IA is halogen. In other embodiments, R⁸ in Formula IA is C₁-C₄ alkyl.

According to the disclosure, n in Formula IA is 0 or 1. In some embodiments, n in Formula IA=0. In other embodiments, n in Formula IA=1.

According to the disclosure, each R⁵ in Formula IA is independently H, halogen, oxo, —OH, —CN, —NO₂, —C₁-C₆alkyl, —C₂-C₆alkenyl, —C₂-C₆alkynyl, haloalkyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(=NR^{b})R^{b}, —SF₅, —P(O)R^{b}R^{b}, —P(O)(OR^{b})(OR^{b}), —B(OR^{d})(OR^{c}) or —S(O)₂R^{b};
each R^{a} is independently H, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)R^{c}R^{b}, —P(O)OR^{c}OR^{b}, —S(O)R^{b}, —S(O)NR^{c}R^{d}, —S(O)₂R^{b}, —S(O)₂NR^{c}R^{d}, SiR^{b}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl;
each R^{b}, is independently H, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl;
each R^{c} or R^{d} is independently H, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; or
R^{c} and R^{d}, together with the atom to which they are both attached, form a monocyclic or multicyclic heterocycloalkyl, or a monocyclic or multicyclic heterocyclo-alkenyl group;

According to the disclosure, R⁵ in Formula IA is independently D, halogen, cyano, C₁-C₄ alkyl, haloalkyl, cyclopropyl, C₁-C₄ alkoxy, haloalkoxy, —O-cyclopropyl, —CH₂—O—CH₃, —C(O)OCH₃, or —C(O)N(H)CH₃. In some embodiments, each R⁵ in Formula IA is D. In some embodiments, at least one R⁵ in Formula IA is halogen. In some embodiments, each R₅ in Formula IA is C₁-C₄ alkoxy. In some embodiments, at least one R⁵ in Formula IA is C₁-C₄ alkoxy.

In other embodiments, at least one R⁵ in Formula IA is fluoro. In other embodiments, at least one R⁵ in Formula IA is cyano. In other embodiments, at least one R⁵ in Formula IA is C₁-C₄ alkyl. In other embodiments, at least one R⁵ in Formula IA is haloalkyl. In other embodiments, at least one R⁵ in Formula IA is cyclopropyl. In other embodiments, at least one R⁵ in Formula IA is haloalkoxy. In other embodiments, at least one R⁵ in Formula IA is —O-cyclopropyl. In other embodiments, at least one R⁵ in Formula IA is —CH₂—O—CH₃. In other embodiments, at least one R⁵ in Formula IA is —C(O)OCH₃. In other embodiments, at least one R⁵ in Formula IA is —C(O)N(H)CH₃.

According to the disclosure, m in Formula IA is 0, 1, 2, 3, or 4. In some embodiments, m is 0. In some embodiments, m in Formula IA=1. In other embodiments, m in Formula IA=2. In other embodiments, m in Formula IA=3. In other embodiments, m in Formula IA=4.

According to the disclosure, each R¹⁰ in Formula IA is independently H, D, halogen, C₁₋₄alk-O—C₁₋₄alkyl, C₁-C₄ alkoxy or C₁-C₄ alkyl. In some embodiments, each R¹⁰ in Formula IA is H. In some embodiments, at least one R¹⁰ in Formula IA is H. In some embodiments, each R¹⁰ in Formula IA is D. In some embodiments, at least one R¹⁰ in Formula IA is D. In some embodiments, each R¹⁰ in Formula IA is halogen. In some embodiments, at least one R¹⁰ in Formula IA is C₁₋₄alk-O—C₁₋₄alkyl. In some embodiments, at least one R¹⁰ in Formula IA is CH₂OCH₃. In some embodiments, at least one R¹⁰ in Formula IA is halogen. In some embodiments, each R¹⁰ in Formula IA is C₁-C₄ alkoxy. In some embodiments, at least one R¹⁰ in Formula IA is C₁-C₄ alkoxy. In some embodiments, each R¹⁰ in Formula IA is C₁-C₄ alkyl. In some embodiments, at least one R¹⁰ in Formula IA is C₁-C₄ alkyl.

According to the disclosure, p in Formula IA is 1, 2, 3, 4, 5, 6, 7 or 8. In some embodiments, p in Formula IA=1. In other embodiments, p in Formula IA=2. In other embodiments, p in Formula IA=3. In other embodiments, p in Formula IA=4. In other embodiments, p in Formula IA=5. In other embodiments, p in Formula IA=6. In other embodiments, p in Formula IA=7. In other embodiments, p in Formula IA=8.

According to the disclosure, PTM is a moiety of Formula IIa:

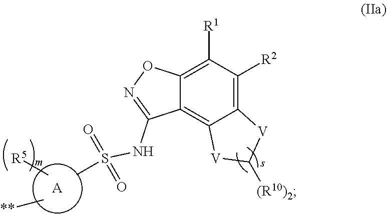

or a pharmaceutically acceptable salt or solvate thereof; wherein R¹, R², R⁵, R⁸, R¹⁰, m, n, p and ring A are as defined herein; and wherein:
each V is independently O, S, NR¹⁰ or C(R¹⁰)₂; and
s is 1, 2, 3 or 4.

According to the disclosure, V in Formula IIa or Formula IIb is O, S, NR¹⁰ or C(R¹⁰)₂. In some embodiments, each V is O. In some embodiments, at least one V is O. In some embodiments, each V is S. In other embodiments, at least one V is S. In other embodiments, each V is NR₁₀. In other embodiments, at least one V is NR¹⁰. In yet other embodiments, each V is C(R¹⁰)₂. In yet other embodiments, at least one V is C(R¹⁰)₂.

In some embodiments, one V in Formula IIa is O and the other V is C(R¹⁰)₂. In some embodiments, both V in Formula IIa are O. In other embodiments, one V in Formula IIa is S and the other V is C(R¹⁰)₂. In other embodiments, both V in Formula IIa are C(R¹⁰)₂.

In some embodiments, V in Formula IIb is O. In some embodiments, V in Formula IIb is S.

According to the disclosure, s in Formula IA is 1, 2, 3 or 4. In some embodiments, s is 1. In some embodiments, s in Formula IA=2. In other embodiments, s in Formula IA=3. In other embodiments, s in Formula IA=4.

According to the disclosure, ULM is a small molecule E3 Ubiquitin Ligase binding moiety that binds a Cereblon E3 Ubiquitin Ligase. In some embodiments, ULM is a moiety as described herein.

Chemical moieties that are used to link PTM and ULM moieties are known in the art. These moieties are sometimes referred to as “linkers” in the art. In some embodiments, Y in Formula IA is a chemical moiety that is used to link a PTM and ULM that is known in the art.

According to the disclosure, Y in Formula IA is a covalent bond, or chemical moiety that links PTM and ULM. In some embodiments, Y in Formula IA is a chemical moiety that links PTM and ULM. In other embodiments, Y in Formula IA is a covalent bond.

In some embodiments, Y in Formula IA is a chemical moiety that is used to link a PTM and ULM as described in U.S. Patent Application Publication No. 2019/0300521, the entirety of which is incorporated by reference herein.

In other embodiments, Y in Formula IA is a chemical moiety that is used to link a PTM and ULM as described in U.S. Patent Application Publication No. 2019/0255066, the entirety of which is incorporated by reference herein.

In other embodiments, Y in Formula IA is a chemical moiety that is used to link a PTM and ULM as described in WO 2019/084030, the entirety of which is incorporated by reference herein.

In other embodiments, Y in Formula IA is a chemical moiety that is used to link a PTM and ULM as described in WO 2019/084026, the entirety of which is incorporated by reference herein.

In some embodiments, Y in Formula IA is a chemical structural unit represented by the formula:

-(G)_{q}-,
wherein:
   q is an integer from 1 to 14;
   each G is independently selected from the group consisting of CR^{1a}R^{1b}, O, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{1c}(CR^{1a}R^{1b})₁₋₄, NR^{1c}C(═NCN)NR^{1d}NR^{1c}C(═NCN), NR^{1c}C(═CNO₂)NR^{1d}, 3-15 membered cycloalkyl, optionally substituted with 1-6 R^{1a} or R^{1b} groups, 3-15 membered heterocyclyl optionally substituted with 1-6 R^{1a} or R^{1b} groups, aryl optionally substituted with 1-6 R^{1a} or R^{1b} groups, heteroaryl optionally substituted with 1-6 R^{1a} or R^{1b} groups,
   and R^{1a}, R^{1b}, R^{1c}, R^{1d} and R^{1e} are each independently, —H, D, -halo, —C₁-C₈alkyl, —C₁-C₆haloalkyl, —O—C₁-C₈alkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; or where the context permits, R^{1a} or R^{1b}, are linked to other groups, or to each other, to form a cycloalkyl and/or a heterocyclyl moiety, optionally substituted with 0-4 R^{1e} groups.

In these embodiments, q represents the number of connected A groups. For example, when q=1, -(G)_{q}- is -G₁-; when q=2, -(G)_{q}- is -G₁-G₂-; when q=3, -(G)_{q}- is -G₁-G₂-G₃-; when q=4, -(G)_{q}- is -G₁-G₂-G₃-G₄-; when q=5, -(G)_{q}- is -G₁-G₂-G₃-G₄-G₅-; when q=6, -(G)_{q}- is -G₁-G₂-G₃-G₄-G₅-G₆-; when q=7, -(G)_{q}- is -G₁-G₂-G₃-G₄-G₅-G₆-G₇-; when q=8, -(G)_{q}- is -G₁-G₂-G₃-G₄-G₅-G₆-G₇-G₅-; when q=9, -(G)_{q}- is -G₁-G₂-G₃-G₄-G₅-G₆-G₇-G₈-G₉-; when q=10, -(G)_{q}- is -G₁-G₂-G₃-G₄-G₅-G₆-G₇-G₅-G₉-G₁₀-; when q=11, -(G)_{q}- is -G₁-G₂-G₃-G₄-G₅-G₆-G₇-G₅-G₉-G₁₀-G₁₁-; when q=12, -(G)_{q}- is -G₁-G₂-G₃-G₄-G₅-G₆-G₇-G₈-G₉-G₁₀-G₁₁-G₁₂-; when q=13, -(G)_{q}- is -G₁-G₂-G₃-G₄-G₅-G₆-G₇-G₅-G₉-G₁₀-G₁₁-G₁₂-G₁₃-; and when q=14, -(G)_{q}- is -G₁-G₂-G₃-G₄-G₅-G₆-G₇-G₈-G₉-G₁₀-G₁₁-G₁₂-G₁₃-G₁₄-.

In some embodiments, q=5 and Y in Formula IA is a chemical moiety represented by the formula: -G₁-G₂-G₃-G₄-G₅-, wherein each of G₁₋₅ is independently selected from the group consisting of O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₄, —(CR^{1a}R^{1b})₁₋₄, —(CR^{1a}R^{1b})₁₋₄NR^{1c}(CR^{1a}R^{1b})₁₋₄, optionally substituted 3-15 membered cycloalkyl, 3-15 membered heterocyclyl, aryl, and heteroaryl;
wherein R^{1a} and R^{1b} are each independently selected from the group consisting of —H, D, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and
R^{1c} and R^{1d} are each independently selected from the group consisting of H, D, optionally substituted C₁₋₄ alkyl, C₃₋₈ cyclcoalkyl, C₃₋₈ heterocyclcoalkyl, aryl, or heteroaryl.

In some embodiments, q=4 and Y in Formula IA is a chemical moiety represented by the formula: -G₁-G₂-G₃-G₄-, wherein each of G₁₋₄ is independently selected from the group consisting of O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR¹a-CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{1c}(CR^{1a}R^{1b})₁₋₄, optionally substituted 3-15 membered cycloalkyl, 3-15 membered heterocyclyl, aryl, and heteroaryl;
wherein R^{1a} and R^{1b} are each independently selected from the group consisting of —H, D, -halo, —C₁-C₈alkyl, —O—C₁-C₈alkyl, —C₁-C₆haloalkyl, —S—C₁-C₈alkyl,—NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and
R^{1c} and R^{1d} are each independently selected from the group consisting of H, D, optionally substituted C₁₋₄ alkyl, C₃₋₈ cyclcoalkyl, C₃₋₈ heterocyclcoalkyl, aryl, or heteroaryl.

In other embodiments, q=3 and Y in Formula IA is a chemical moiety represented by the formula: -G₁-G₂-G₃-, wherein each of G₁₋₃ is independently selected from the group consisting of O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{1c}(CR^{1a}R^{1b})₁₋₄, optionally substituted 3-15 membered cycloalkyl, 3-15 membered heterocyclyl, aryl, and heteroaryl;
wherein R^{1a} and R^{1b} are each independently selected from the group consisting of —H, D, -halo, —C₁-C₈alkyl, —O—C₁-C₈alkyl, —C₁-C₆haloalkyl, —S—C₁-C₈alkyl,—NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and
R^{1c} and R^{1d} are each independently selected from the group consisting of H, D, optionally substituted C₁₋₄ alkyl, C₃₋₈ cyclcoalkyl, C₃₋₈ heterocyclcoalkyl, aryl, or heteroaryl.

In other embodiments, q=2 and Y in Formula IA is a chemical moiety represented by the formula: -G₁-G₂-, wherein each of G₁₋₂ is independently selected from the group consisting of O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{1c}(CR^{1a}R^{1b})₁₋₄, optionally substituted 3-15 membered cycloalkyl, 3-15 membered heterocyclyl, aryl, and heteroaryl;
wherein R^{1a} and R^{1b} are each independently selected from the group consisting of —H, D, -halo, —C₁-C₈alkyl, —O—C₁-C₈alkyl, —C₁-C₆haloalkyl, —S—C₁-C₈alkyl,—NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and
R^{1c} and R^{1d} are each independently selected from the group consisting of H, D, optionally substituted C₁₋₄ alkyl, C₃₋₈ cyclcoalkyl, C₃₋₈ heterocyclcoalkyl, aryl, or heteroaryl.

In other embodiments, q=1 and Y in Formula IA is a chemical moiety represented by the formula: -G₁-, wherein G₁ is selected from the group consisting of O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}—CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{1c}(CR^{1a}R^{1b})₁₋₄, optionally substituted 3-15 membered cycloalkyl, 3-15 membered heterocyclyl, aryl, and heteroaryl;
wherein R^{1a} and R^{1b} are each independently selected from the group consisting of —H, D, -halo, —C₁-C₈alkyl, —O—C₁-C₈alkyl, —C₁-C₆haloalkyl, —S—C₁-C₈alkyl,—NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and
R^{1c} and R^{1d} are each independently selected from the group consisting of H, D, optionally substituted C₁₋₄ alkyl, C₃₋₈ cyclcoalkyl, C₃₋₈ heterocyclcoalkyl, aryl, or heteroaryl.

In some embodiments, Y in Formula IA is a covalent bond, 3-15 membered cycloalkyl optionally substituted with 1-6 R^{1a} or R^{1b} groups, 3-15 membered heterocyclyl optionally substituted with 1-6 R^{1a} or R^{1b} groups, —(CR^{1a}R^{1b})₁₋₅, —(CR^{1a}—CR^{1b})—, —(CR^{1a}R^{1b})₁₋₅-G- wherein G is O, S, or NR^{1c}, —(CR^{1a}R^{1b})₁₋₅-G-(CR^{1a}R^{1b})₁₋₅- wherein G is O, S, or NR^{1c}, —(CR^{1a}R^{1b})₁₋₅-G-(CR^{1a}R^{1b})₁₋₅-G- wherein G is O, S, or NR^{1c}, —(CR^{1a}R^{1b})₁₋₅—(CR^{1a}=CR^{1b})—(CR^{1a}R^{1b})₁₋₅—, —(CR^{1a}R^{1b})₁₋₅—(CR^{1a}—CR^{1b})—(CR^{1a}R^{1b})₁₋₅-G- wherein G is O, S, or NR^{1c}, —(CR^{1a}R^{1b})₁₋₅—(C≡C)—(CR^{1a}R^{1b})₁₋₅—, —(CR^{1a}R^{1b})₁₋₅—(C—C)—(CR^{1a}R^{1b})₁-s-G- wherein G is O, S, or NR^{1c}, —(C≡C)—(CR^{1a}R^{1b})₁₋₅-G-(CR^{1a}R^{1b})₁₋₅- wherein G is O, S, or NR^{1c}, —(C—C)—(CR^{1a}R^{1b})₁₋₅, —(CR^{1a}R^{1b})₁₋₅-(3-15 membered cycloalkyl optionally substituted with 1-6 R^{1a} or R^{1b} groups)-, —(CR^{1a}R^{1b})₁₋₅-(3-15 membered heterocyclyl optionally substituted with 1-6 R^{1a} or R^{1b} groups)-, -(3-15 membered cycloalkyl optionally substituted with 1-6 R^{1a} or R^{1b} groups)-(CR^{1a}R^{1b})₁₋₅—, -(3-15 membered heterocyclyl optionally substituted with 1-6 R^{1a} or R^{1b} groups)-(CR^{1a}R^{1b})₁₅—, —(CR^{1a}R^{1b})₁₋₅-(3-15 membered cycloalkyl optionally substituted with 1-6 R^{1a} or R^{1b} groups)-G-, —(CR^{1a}R^{1b})₁₋₅-(3-15 membered heterocyclyl optionally substituted with 1-6 R^{1a} or R^{1b} groups)-G-, —(CR^{1a}R^{1b})₁₋₅-(3-15 membered cycloalkyl optionally substituted with 1-6 R^{1a} or R^{1b} groups)-(CR^{1a}R^{1b})₁₅, —(CR^{1a}R^{1b})₁₋₅-(3-15 membered cycloalkyl optionally substituted with 1-6 R^{1a} or R^{1b} groups)-G- wherein G is O, S, or NR^{1c}, —(CR^{1a}R^{1b})₁₋₅-(3-15 membered cycloalkyl optionally substituted with 1-6 R^{1a} or R^{1b} groups)-(CR^{1a}R^{1b})₁₋₅-G- wherein G is O, S, or NR^{1c}, —(CR^{1a}R^{1b})₁₋₅-G-(3-15 membered cycloalkyl optionally substituted with 1-6 R^{1a} or R^{1b} groups)- wherein G is O, S, or NR^{1c}, —(CR^{1a}R^{1b})₁₋₅-(3-15 membered heterocyclyl optionally substituted with 1-6 R^{1a} or R^{1b} groups)-(CR^{1a}R^{1b})₁₋₅, —(CR^{1a}R^{1b})₁₋₅-(3-15 membered heterocyclyl optionally substituted with 1-6 R^{1a} or R^{1b} groups)-(CR^{1a}R^{1b})₁₋₅-G- wherein G is O, S, or NR^{1c}, —(CR^{1a}R^{1b})₁₋₅-(3-15 membered heterocyclyl optionally substituted with 1-6 R^{1a} or R^{1b} groups)-G- wherein G is O, S, or NR^{1c}, —(CR^{1a}R^{1b})₁₋₅-G-(3-15 membered heterocyclyl optionally substituted with 1-6 R^{1a} or R^{1b} groups)- wherein G is O, S, or NR^{1c}, —(CR^{1a}R^{1b})₁₋₅-(3-15 membered cycloalkyl optionally substituted with 1-6 R^{1a} or R^{1b} groups)-(CR^{1a}R^{1b})i-s-G- wherein G is O, S, or NR^{1c}, —(CR^{1a}R^{1b})₁₋₅-G-(3-15 membered cycloalkyl optionally substituted with 1-6 R^{1a} or R^{1b} groups)- wherein G is O, S, or NR^{1c}, —(CR^{1a}R^{1b})₁₋₅-G-(3-15 membered cycloalkyl optionally substituted with 1-6 R^{1a} or R^{1b} groups)-(CR^{1a}R^{1b})₁₋₅-G- wherein each G is independently O, S, or NR^{1c}, —(CR^{1a}R^{1b})₁₋₅-G-(3-15 membered heterocyclyl optionally substituted with 1-6 R^{1a} or R^{1b} groups)-(CR^{1a}R^{1b})₁₋₅-G- wherein each G is independently O, S, or NR^{1c}, —(CR^{1a}R^{1b})₁₋₅-G-(CR^{1a}R^{1b})₁₋₅-G- wherein G is O, S, or NR^{1c}, —(CR^{1a}R^{1b})₁₋₅-G-(CR^{1a}R^{1b})₁₋₅-G-(CR^{1a}R^{1b})₁₋₅-G-(CR^{1a}R^{1b})₁₋₅-G- wherein G is O, S, or NR^{1c}, —(CR^{1a}R^{1b})₁₋₅-G-(CO) wherein G is O, S, or NR^{1c}, —(CR^{1a}R^{1b})₁₋₅—(CR^{1a}=CR^{1b})—(CR^{1a}R^{1b})₁₋₅-G-(CO)— wherein G is O, S, or NR^{1c}, —(CR^{1a}R^{1b})₁₋₅—(C≡C)—(CR^{1a}R^{1b})₁₋₅-G-(CO)— wherein G is O, S, or NR^{1c}, —(CR^{1a}R^{1b})₁₋₅-(3-15 membered cycloalkyl optionally substituted with 1-6 R^{1a} or R^{1b} groups)-(CR^{1a}R^{1b})₁₋₅-G-(CO)— wherein G is O, S, or NR^{1c}, —(CR^{1a}R^{1b})₁₋₅-G-(CO)-(3-15 membered cycloalkyl optionally substituted with 1-6 R^{1a} or R^{1b} groups)- wherein G is O, S, or NR^{1c}, —(CR^{1a}R^{1b})₁₋₅-(3-15 membered heterocyclyl optionally substituted with 1-6 R^{1a} or R^{1b} groups)-(CR^{1a}R^{1b})₁₋₅-G-(CO)— wherein G is O, S, or NR^{1c}, —(CR^{1a}R^{1b})₁₋₅-G-(CO)-(3-15 membered heterocyclyl optionally substituted with 1-6 R^{1a} or R^{1b} groups)- wherein G is O, S, or NR^{1c}, —(CR^{1a}R^{1b})₁₋₅-G-(3-15 membered cycloalkyl optionally substituted with 1-6 R^{1a} or R^{1b} groups)-G-(CO)— wherein each G is independently O, S, or NR^{1c}, -(3-15 membered cycloalkyl optionally substituted with 1-6 R^{1a} or R^{1b} groups)-CO—(CR^{1a}R^{1b})₁₋₅-G- wherein G is O, S, or NR^{1c}, —(CR^{1a}R^{1b})₁₋₅-(3-15 membered cycloalkyl optionally substituted with 1-6 R^{1a} or R^{1b} groups)-(CR^{1a}R^{1b})₁₋₅-G-(CO)— wherein G is O, S, or NR^{1c}, —(CR^{1a}R^{1b})₁₋₅-(3-15 membered heterocyclyl optionally substituted with 1-6 R^{1a} or R^{1b} groups)-(CR^{1a}R^{1b})₁₋₅-G-(CO)— wherein G is O, S, or NR^{1c}, -(3-15 membered cycloalkyl optionally substituted with 1-6 R^{1a} or R^{1b} groups)-(CR^{1a}R^{1b})₁₋₅—, or -(3-15 membered heterocyclyl optionally substituted with 1-6 R^{1a} or R^{1b} groups)-(CR^{1a}R^{1b})₁—.

In some embodiments, Y in Formula IA is —CR^{1a}—CR^{1b}—, such as, for example, —CH═CH—.

In some embodiments, Y in Formula IA is —(CR^{1a}R^{1b})₁₋₅, for example —(CH₂)₁₋₅—, —CH₂—, —CH₂CH₂CH₂— and the like.

In some embodiments, Y in Formula IA is —(CR^{1a}R^{1b})₁₋₅-G- wherein G is O, S, or NR^{1c}, such as for example, —(CH₂)₁₋₅—O—, —(CH₂)₁₋₅—S—, —(CH₂)₁₋₅—NH—, or —(CH₂)₀₋₂—(C(CH₃)₂)—(CH₂)₀₋₂—O—.

In other embodiments, Y in Formula IA is —(CR^{1a}R^{1b})₁₋₅-G-(CR^{1a}R^{1b})₁₋₅- wherein G is O, S, or NR^{1c}, such as, for example, —(CH₂)₁₋₅—O—(CH₂)₁₋₅—, —(CH₂)₁₋₅—S—(CH₂)₁₋₅—, —(CH₂)₁₋₅—NH—(CH₂)₁₋₅—.

In some embodiments, Y in Formula IA is —(C≡C)—(CR^{1a}R^{1b})₁₋₅, such as, for example, —(C≡C)—(CH₂)₂—, and the like.

In some embodiments, Y in Formula IA is —(CR^{1a}R^{1b})₁₋₅-(3-15 membered cycloalkyl optionally substituted with 1-6 R^{1a} or R^{1b} groups)-, such as, for example, —CH₂-cyclobutyl-.

In some embodiments, Y in Formula IA is —(CR^{1a}R^{1b})₁₋₅-(3-15 membered cycloalkyl optionally substituted with 1-6 R^{1a} or R^{1b} groups)-(CR^{1a}R^{1b})₁₅, such as, for example, —CH₂— cyclobutyl-CH₂— and the like.

In some embodiments, Y in Formula IA is —(CR^{1a}R^{1b})₁₋₅-(3-15 membered heterocyclyl optionally substituted with 1-6 R^{1a} or R^{1b} groups)-(CR^{1a}R^{1b})₁₋₅, such as, for example, —CH₂-azetidinyl-CH₂—.

In some embodiments, Y in Formula IA is —(CR^{1a}R^{1b})₁₋₅-(3-15 membered heterocyclyl optionally substituted with 1-6 R^{1a} or R^{1b} groups)-, such as, for example, —CH₂— azetidinyl-.

In some embodiments, Y in Formula IA is -(3-15 membered heterocyclyl optionally substituted with 1-6 R^{1a} or R^{1b} groups) —(CR^{1a}R^{1b})₁₋₅, such as, for example, -azetidinyl-CH₂—, -pyrolidinyl-CH₂—, -piperidinyl-CH₂—, and the like.

In some embodiments, Y in Formula IA is —(CR^{1a}R^{1b})₁₋₅-(3-15 membered cycloalkyl optionally substituted with 1-6 R^{1a} or R^{1b} groups)-(CR^{1a}R^{1b})₁₋₅-G- wherein G is O, S, or NR^{1c}, such as, for example, —CH₂-cyclopropyl-CH₂—O—, and the like.

In some embodiments, Y in Formula IA is —(CR^{1a}R^{1b})₁₋₅-(3-15 membered heterocyclyl optionally substituted with 1-6 R^{1a} or R^{1b} groups)-(CR^{1a}R^{1b})₁₋₅-G- wherein G is O, S, or NR^{1c}, such as, for example, —CH₂-piperidinyl-CH₂CH₂—O—, and the like.

In some embodiments, Y in Formula IA is —(CR^{1a}R^{1b})₁₋₅-(3-15 membered heterocyclyl optionally substituted with 1-6 R^{1a} or R^{1b} groups)-G- wherein G is O, S, or NR^{1c}, such as, for example, —CH₂-azetidinyl-O—, and the like.

In some embodiments, Y in Formula IA is —(CR^{1a}R^{1b})₁₋₅-G-(3-15 membered heterocyclyl optionally substituted with 1-6 R^{1a} or R^{1b} groups)- wherein G is O, S, or NR^{1c}, such as, for example, —CH₂—O-azetidinyl-, —CH₂—NH-azetidinyl-, and the like.

In other embodiments, Y in Formula IA is —(CR^{1a}R^{1b})₁₋₅-G-(3-15 membered cycloalkyl optionally substituted with 1-6 R^{1a} or R^{1b} groups)- wherein G is O, S, or NR^{1c}, such as —CH₂—O-cyclobutylene-, —CH₂—NH-cyclobutylene-, and the like.

In some embodiments, Y in Formula IA is —(CR^{1a}R^{1b})₁₋₅-G-(CR^{1a}R^{1b})₁₋₅-G- wherein G is O, S, or NR^{1c}, such as, for example, —CH₂—O—CH₂CH₂—O—.

In some embodiments, Y in Formula IA is

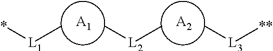

wherein
** is a point of attachment to Ring A of the PTM;
* is a point of attachment to ULM;
L₁, L₂, and L₃ are each independently a bond, CR^{1a}R^{1b}, O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}=CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{1c}(CR^{1a}R^{1b})₁₋₄, NR^{1c}C(═NCN)NR^{1d}, NR^{1c}C(═NCN), NR^{1c}C(═CNO₂)NR^{1d};
ring A₁ and ring A₂ are each independently 3-15 membered cycloalkyl, optionally substituted with 1-6 R^{1a} or R^{1b} groups, 3-15 membered heterocyclyl optionally substituted with 1-6 R^{1a} or R^{1b} groups, aryl optionally substituted with 1-6 R^{1a} or R^{1b} groups, or heteroaryl optionally substituted with 1-6 R^{1a} or R^{1b} groups,
wherein R^{1a}, R^{1b}, R^{1c}, R^{1d} and R^{1e} are each independently, —H, D, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and where R^{1a} or R^{1b}, each independently may be optionally linked to other groups to form cycloalkyl and/or heterocyclyl moiety, optionally substituted with 0-4 R^{1e} groups.

In some embodiments, Y in Formula IA is

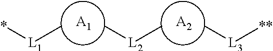

wherein
** is a point of attachment to PTM;
L₁ is a bond, (C(R¹⁰)₂)ₚ or CO;
L₂ is a bond, (C(R¹⁰)₂)ₚ or CO;
L₃ is a bond, (C(R¹⁰)₂)ₚ or CO;
each p is independently 1, 2, 3, or 4;
each R¹⁰ is independently H, D, or C₁-C₄ alkyl;
ring A₁ is a 3-7 membered cycloalkyl group, a 4-10-membered heterocycloalkyl group, an aryl group, or a heteroaryl group; and
ring A₂ is a 4-15 membered cycloalkyl group, a 4-15-membered heterocycloalkyl group, an aryl group, or a heteroaryl group.

According to the disclosure, L₁ is a bond, (C(R¹⁰)₂)ₚ or CO. In some embodiments, L₁ is a bond. In some embodiments, L₁ is (C(R¹⁰)₂)ₚ. In other embodiments, L₁ is CO.

According to the disclosure, L₂ is a bond, (C(R¹⁰)₂)ₚ or CO. In some embodiments, L₂ is a bond. In some embodiments, L₂ is (C(R¹⁰)₂)ₚ. In other embodiments, L₂ is CO.

According to the disclosure, L₃ is a bond, (C(R¹⁰)₂)ₚ or CO. In some embodiments, L₃ is a bond. In some embodiments, L₃ is (C(R¹⁰)₂)ₚ. In other embodiments, L₃ is CO.

According to the disclosure, each p is independently 1, 2, 3, or 4. In some embodiments, each p is 1. In some embodiments, at least one p is 1. In some embodiments, each p is 2. In some embodiments, at least one p is 2. In other embodiments, each p is 3. In other embodiments, at least one p is 3. In other embodiments, each p is 4. In other embodiments, at least one p is 4.

According to the disclosure, each R¹⁰ is independently H, D, or C₁-C₄ alkyl. In some embodiments, each R¹⁰ is H. In some embodiments, at least one R¹⁰ is H. In some embodiments, each R¹⁰ is D. In some embodiments, at least one R¹⁰ is D. In other embodiments, each R¹⁰ is C₁-C₄ alkyl. In other embodiments, at least one R¹⁰ is C₁-C₄ alkyl. In other embodiments, each R¹⁰ is methyl or ethyl. In other embodiments, at least one R¹⁰ is methyl or ethyl.

According to the disclosure, ring A₁ is a 3-15 membered cycloalkyl group, a 4-15 membered heterocycloalkyl group, an aryl group, or a heteroaryl group. In some embodiments, ring A₁ is a 3-15 membered cycloalkyl group. In some embodiments, ring A₁ is a 4-15 membered heterocycloalkyl group. In other embodiments, ring A₁ is an aryl group. In other embodiments, ring A₁ is a heteroaryl group.

In some embodiments, ring A₁ is a piperazine group, a morpholine group, a piperidine group, a pyrrolidine group, an azetidine group or an azabicyclo-alkyl group. In other embodiments, ring A₁ is a piperidine or pyrrolidine group.

According to the disclosure, ring A₂ is a 3-15 membered cycloalkyl group, a 4-15 membered heterocycloalkyl group, an aryl group, or a heteroaryl group. In some embodiments, ring A₂ is a 3-15 membered cycloalkyl group. In some embodiments, ring A₂ is a 4-15 membered heterocycloalkyl group. In other embodiments, ring A₂ is an aryl group. In other embodiments, ring A₂ is a heteroaryl group.

In some embodiments, ring A₂ is a piperazine group, a morpholine group, a piperidine group, a pyrrolidine group, an azetidine group, a diazaspiroalkyl group or an azabicycloalkyl group. In other embodiments, ring A₂ is a piperazine group or a diazaspirononane group.

In some embodiments, Y in Formula IA is

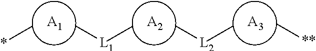

wherein
** is a point of attachment to Ring A of the PTM;
*is a point of attachment to ULM;
L₁ and L₂ are each independently a bond, CR^{1a}R^{1b}, O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{1c}(CR^{1a}R^{1b})₁₋₄, NR^{1c}C(═NCN)NR^{1d}, NR^{1c}C(═NCN), NR^{1c}C(═CNO₂)NR^{1d};
ring A₁, ring A₂ and ring A₃ are each independently 3-15 membered cycloalkyl, optionally substituted with 1-6 R^{1a} or R^{1b} groups, 3-15 membered heterocyclyl optionally substituted with 1-6 R^{1a} or R^{1b} groups, aryl optionally substituted with 1-6 R^{1a} or R^{1b} groups, or heteroaryl optionally substituted with 1-6 R^{1a} or R^{1b} groups,
wherein R^{1a}, R^{1b}, R^{1c}, R^{1d} and R^{1e} are each independently, —H, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂.

In some embodiments, Y in Formula IA is

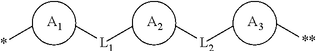

wherein
** is a point of attachment to Ring A of the PTM;
* is a point of attachment to ULM;
L₁ is a bond, (C(R¹⁰)₂)ₚ, or CO;
L₂ is a bond, (C(R¹⁰)₂)ₚ, or CO;
p is 1, 2, 3 or 4;
each R¹⁰ is independently H or C₁-C₄ alkyl;
ring A₁ is a 3-7 membered cycloalkyl group, a 4-10-membered heterocycloalkyl group, an aryl group, or a heteroaryl group; and
ring A₂ is a 3-15 membered cycloalkyl group, a 4-15 membered heterocycloalkyl group, an aryl group, or a heteroaryl group; and
ring A₃ is a 3-15 membered cycloalkyl group, a 4-15 membered heterocycloalkyl group, an aryl group, or a heteroaryl group.

According to the disclosure, L₁ is a bond, (C(R¹⁰)₂)ₚ or CO. In some embodiments, L₁ is a bond. In some embodiments, L₁ is (C(R¹⁰)₂)ₚ. In other embodiments, L₁ is CO.

According to the disclosure, L₂ is a bond, (C(R¹⁰)₂)ₚ or CO. In some embodiments, L₂ is a bond. In some embodiments, L₂ is (C(R¹⁰)₂)ₚ. In other embodiments, L₂ is CO.

According to the disclosure, each p is independently 1, 2, 3, or 4. In some embodiments, each p is 1. In some embodiments, at least one p is 1. In some embodiments, each p is 2. In some embodiments, at least one p is 2. In other embodiments, each p is 3. In other embodiments, at least one p is 3. In other embodiments, each p is 4. In other embodiments, at least one p is 4.

According to the disclosure, each R¹⁰ is independently H, D, or C₁-C₄ alkyl. In some embodiments, each R¹⁰ is H. In some embodiments, at least one R¹⁰ is H. In some embodiments, each R¹⁰ is D. In some embodiments, at least one R¹⁰ is D. In other embodiments, each R¹⁰ is C₁-C₄ alkyl. In other embodiments, at least one R¹⁰ is C₁-C₄ alkyl. In other embodiments, each R¹⁰ is methyl or ethyl. In other embodiments, at least one R¹⁰ is methyl or ethyl.

According to the disclosure, ring A₁ is a 3-15 membered cycloalkyl group, a 4-15 membered heterocycloalkyl group, an aryl group, or a heteroaryl group. In some embodiments, ring A₁ is a 3-15 membered cycloalkyl group. In some embodiments, ring A₁ is a 4-15 membered heterocycloalkyl group. In other embodiments, ring A₁ is an aryl group. In other embodiments, ring A₁ is a heteroaryl group.

In some embodiments, ring A₁ is a piperazine group, a morpholine group, a piperidine group, a pyrrolidine group, an azetidine group or an azabicyclo-alkyl group. In other embodiments, ring A₁ is a piperidine or pyrrolidine group.

According to the disclosure, ring A₂ is a 3-15 membered cycloalkyl group, a 4-15 membered heterocycloalkyl group, an aryl group, or a heteroaryl group. In some embodiments, ring A₂ is a 3-15 membered cycloalkyl group. In some embodiments, ring A₂ is a 4-15 membered heterocycloalkyl group. In other embodiments, ring A₂ is an aryl group. In other embodiments, ring A₂ is a heteroaryl group.

In some embodiments, ring A₂ is a piperazine group, a morpholine group, a piperidine group, a pyrrolidine group, an azetidine group, a diazaspiroalkyl group or an azabicycloalkyl group. In other embodiments, ring A₂ is a piperazine group or a diazaspirononane group.

According to the disclosure, ring A₃ is a 3-15 membered cycloalkyl group, a 4-15 membered heterocycloalkyl group, an aryl group, or a heteroaryl group. In some embodiments, ring A₃ is a 3-15 membered cycloalkyl group. In some embodiments, ring A₃ is a 4-15 membered heterocycloalkyl group. In other embodiments, ring A₃ is an aryl group. In other embodiments, ring A₃ is a heteroaryl group.

In some embodiments, ring A₃ is a piperazine group or a piperidine group. In some embodiments, ring A₃ is a piperazine group. In some embodiments, ring A₃ is a piperidine group.

In some embodiments, Y in Formula IA is

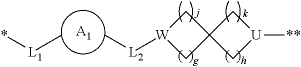

wherein
W is C(R¹⁵) or N;
U is C(R¹⁵) or N;
R¹⁵ is H, F, C₁₋₄alkyl or C₁₋₄alkoxide;
each g and h is independently 1, 2 or 3; and
each j and k is independently 0, 1, 2 or 3.

In some embodiments, W is C(R¹⁵). In some embodiments, W is CH. In other embodiments, W is N. In some embodiments, U is CR₁₅. In other embodiments, U is N. In some embodiments, both U and W are N.

In some embodiments, R¹⁵ is H. In some embodiments, R₁₅ is halogen. In other embodiments, R¹⁵ is F. In some embodiments, R¹⁵ is C₁₋₆alkyl. In other embodiments, R¹⁵ is methyl or ethyl. In some embodiments, R¹⁵ is C₁₋₆alkoxide.

In some embodiments, g is 1, 2 or 3. In some embodiments, g is 1. In other embodiments, g is 2. In other embodiments, g is 3.

In some embodiments, h is 1, 2 or 3. In some embodiments, h is 1. In other embodiments, h is 2. In other embodiments, h is 3.

In some embodiments, j is 0, 1, 2 or 3. In some embodiments, j is 0. In some embodiments, j is 1. In other embodiments, j is 2. In other embodiments, j is 3.

In some embodiments, k is 0, 1, 2 or 3. In some embodiments, k is 0. In some embodiments, k is 1. In other embodiments, k is 2. In other embodiments, k is 3.

In some embodiments, j and g are each 2. In some embodiments, k and h are each 1.

According to the disclosure, ULM in Formula I is

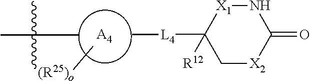

wherein:
[symbol] is a point of attachment to Y or PTM;
Ring A₄ is a monocyclic, bicyclic or tricyclic aryl, heteroaryl or heterocycle group,
L₄ is a bond, —O—, —S—, —NR^{a}—, —C(R^{a})₂— —C(O)NR^{a}—;
X₁ is CH₂, CO, CH═CH (when X₂═CO), or N═CH (when X₂═CO);
X₂ is CH₂, CO, CH═CH (when X₁═CO), or N═CH (when X₁═CO);
R¹² is H, D, optionally substituted C₁₋₄ alkyl, C₁₋₄ alkoxyl, C₁₋₄haloalkyl, —CN, —OR^{a}, —OR^{b} or —SR^{b};
each R²⁵ is independently H, D, halogen, oxo, —OH, —CN, —NO₂, —C₁-C₆alkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{d})(OR^{c}) or —S(O)₂R^{b};
each R^{a} is independently H, D, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)R—R^{b}, —P(O)OR—OR^{b}, —S(O)R^{b}, —S(O)NR^{c}R^{d}, —S(O)₂R^{b}, —S(O)₂NR^{c}R^{d}, SiR^{b}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl;
each R^{b}, is independently H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl;
each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; or
R^{c} and R^{d}, together with the atom to which they are both attached, form a monocyclic or multicyclic heterocycloalkyl, or a monocyclic or multicyclic heterocyclo-alkenyl group; and
is 1, 2, 3, 4, or 5.

According to the disclosure, Ring A₄ is a monocyclic, bicyclic or tricyclic aryl, heteroaryl or heterocycle group. In some embodiments, Ring A₄ is a monocyclic, bicyclic or tricyclic aryl group. In other embodiments, Ring A₄ is a monocyclic, bicyclic or tricyclic heteroaryl group. In other embodiments, Ring A₄ is a monocyclic, bicyclic or tricyclic heterocycle group.

In some embodiments, Ring A₄ is a bicyclic heterocycle group. In some embodiments, Ring A₄ is an isoindoline group. In other embodiments, Ring A₄ is an isoindolin-1-one group. In other embodiments, Ring A₄ is an isoindolin-3-one group. In other embodiments, Ring A₄ is an isoindoline-1,3-dione group.

According to the disclosure, each R²⁵ is independently H, D, halogen, oxo, —OH, —CN, —NO₂, —C₁-C₆alkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR—R^{d}, —S(O)R^{b}, —S(O)₂NR—R^{d}, —S(O)(═NR^{b})R^{b}, —SF₅, —P(O)R^{b}R^{b}, —P(O)(OR^{b})(OR^{b}), —B(OR^{d})(OR^{c}) or —S(O)₂R^{b}.

In some embodiments, each R²⁵ is H. In some embodiments, at least one R²⁵ is H. In some embodiments, each R²⁵ is D. In some embodiments, at least one R²⁵ is D. In some embodiments, each R²⁵ is C₁-C₆alkyl. In some embodiments, at least one R²⁵ is C₁-C₆alkyl. In some embodiments, each R²⁵ is methyl or ethyl. In some embodiments, at least one R²⁵ is methyl or ethyl.

In other embodiments, each R²⁵ is independently selected from halogen, oxo, —OH, —CN, —NO₂, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{C}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{d})(OR^{c}) or —S(O)₂R^{b}.

According to the disclosure, o is 1, 2, 3, 4, or 5. In some embodiments, o is 1. In some embodiments, o is 2. In other embodiments, o is 3. In other embodiments, o is 4. In other embodiments, o is 5.

According to the disclosure, L₄ is a bond, —O—, —S—, —NR^{a}—, —C(R^{a})₂— —C(O)NR^{a}—. In some embodiments, L₄ is a bond. In some embodiments, L₄ is —O—. In other embodiments, L₄ is a —S—. In other embodiments, L₄ is —NR^{a}—. In other embodiments, L₄ is —C(R^{a})₂—. In other embodiments, L₄ is —C(O)NR^{a}—.

According to the disclosure, X₁ is CH₂, CO, CH═CH (when X₂═CO), or N═CH (when X₂═CO). In some embodiments, X₁ is CH₂. In some embodiments, X₁ is CO. In other embodiments, X₁ is CH═CH (when X₂═CO). In other embodiments, X₁ is N═CH (when X₂═CO).

According to the disclosure, X₂ is CH₂, CO, CH═CH (when X₁═CO), or N═CH (when X₁═CO). In some embodiments, X₂ is CH₂. In some embodiments, X₂ is CO. In other embodiments, X₂ is CH═CH (when X₁═CO). In other embodiments, X₂ is N═CH (when X₁═CO).

According to the disclosure, R¹² is H, D, optionally substituted C₁₋₄ alkyl, C₁₋₄ alkoxyl, C₁₋₄haloalkyl, —CN, —OR^{a}, —OR^{b} or —SR^{b}. In some embodiments, R¹² is H. In some embodiments, R¹² is D. In some embodiments, R¹² is optionally substituted C₁₋₄ alkyl. In other embodiments, R¹² is C₁₋₄ alkoxyl. In other embodiments, R¹² is C₁₋₄haloalkyl. In other embodiments, R¹² is —CN. In other embodiments, R¹² is —OR^{a}. In other embodiments, R¹² is —OR^{b}. In other embodiments, R¹² is —SR^{b}.

According to the disclosure, ULM in Formula I is

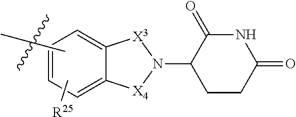

wherein:
[symbol] is a point of attachment to Y or PTM;
R²⁵ is as defined herein;
X₃ is CH₂, CO, CH═CH (when X₄═CO), or N═CH (when X₄═CO); and
X₄ is CH₂, CO, CH═CH (when X₃═CO), or N═CH (when X₃═CO).

According to the disclosure, X₃ is CH₂, CO, CH═CH (when X₄═CO), or N═CH (when X₄═CO). In some embodiments, X₃ is CH₂. In some embodiments, X₃ is CO. In other embodiments, X₃ is CH═CH (when X₄═CO). In other embodiments, X₃ is N═CH (when X₄═CO).

According to the disclosure, X₄ is CH₂, CO, CH═CH (when X₃═CO), or N═CH (when X₃═CO). In some embodiments, X₄ is CH₂. In some embodiments, X₄ is CO. In other embodiments, X₄ is CH═CH (when X₃═CO). In other embodiments, X₄ is N═CH (when X₃═CO).

According to the disclosure, Y-PTM is a compound of Formula IIIa or Formula IIIb:

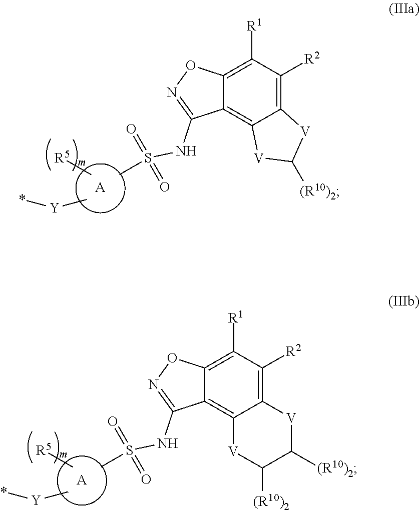

or a pharmaceutically acceptable salt thereof; wherein * is a point of attachment to ULM;
and wherein V, R¹, R², R⁵, R⁸, R¹⁰, m, and ring A are as defined herein.

According to the disclosure, Y-PTM is a compound of Formula IVa or Formula IVb:

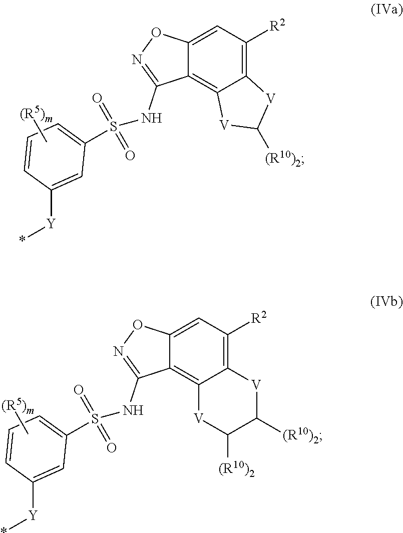

or a pharmaceutically acceptable salt thereof; wherein * is a point of attachment to ULM;
and wherein V, R¹, R², R⁵, R⁸, R¹⁰ and m are as defined herein.

According to the disclosure, Y-PTM is a compound of Formula Va or Formula Vb:

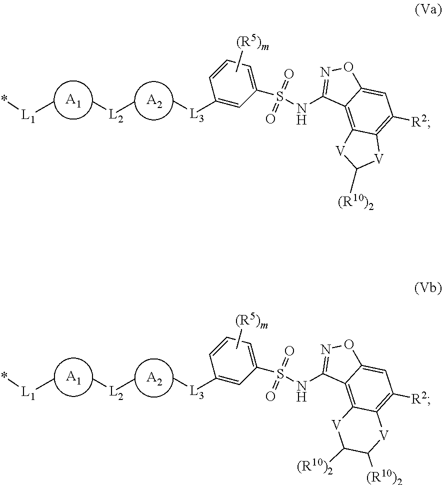

or a pharmaceutically acceptable salt thereof; wherein
   * is a point of attachment to ULM;
   L₁ is a bond, (C(R¹⁰)₂)ₚ, or CO;
   L₂ is a bond, (C(R¹⁰)₂)ₚ, or CO;
   L₃ is a bond, (C(R¹⁰)₂)ₚ, or CO;
   p is 1, 2, 3 or 4;
   each R¹⁰ is independently H or C₁-C₄ alkyl;
   ring A₁ is a 3-15 membered cycloalkyl group, a 4-15-membered heterocycloalkyl group, an aryl group, or a heteroaryl group; and
   ring A₂ is a 3-15 membered cycloalkyl group, a 4-15-membered heterocycloalkyl group, an aryl group, or a heteroaryl group.

In some embodiments, at least one of ring A₁ and ring A₂ in Formula Va or Formula Vb is a 4-15-membered heterocycloalkyl group. In some embodiments, at least one of ring A₁ and ring A₂ in Formula Va or Formula Vb is

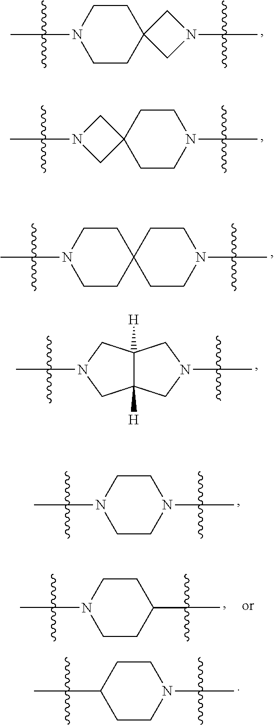

In some embodiments, at least one of ring A₁ and ring A₂ in Formula Va or Formula Vb is

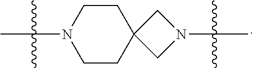 [SMILES: *N1CCC2(CC1)CN(*)C2]

In some embodiments, at least one of ring A₁ and ring A₂ in Formula Va or Formula Vb is

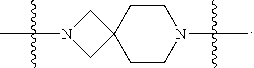 [SMILES: *N1CCC2(CC1)CN(*)C2]

In other embodiments, at least one of ring A₁ and ring A₂ in Formula Va or Formula Vb is

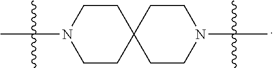 [SMILES: *N1CCC2(CC1)CCN(*)CC2]

In other embodiments, at least one of ring A₁ and ring A₂ in Formula Va or Formula Vb is

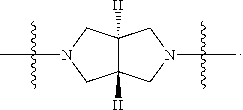 [SMILES: *N1C[C@H]2CN(*)C[C@@H]2C1]

In other embodiments, at least one of ring A₁ and ring A₂ in Formula Va or Formula Vb is

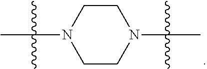 [SMILES: *N1CCN(*)CC1]

In yet other embodiments, at least one of ring A₁ and ring A₂ in Formula Va or Formula Vb is

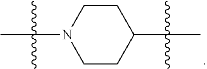 [SMILES: *C1CCN(*)CC1]

In yet other embodiments, at least one of ring A₁ and ring A₂ in Formula Va or Formula Vb is

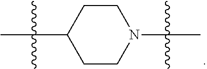 [SMILES: *C1CCN(*)CC1]

According to the disclosure, a compound of Formula Va is a compound of Formula Va-1:

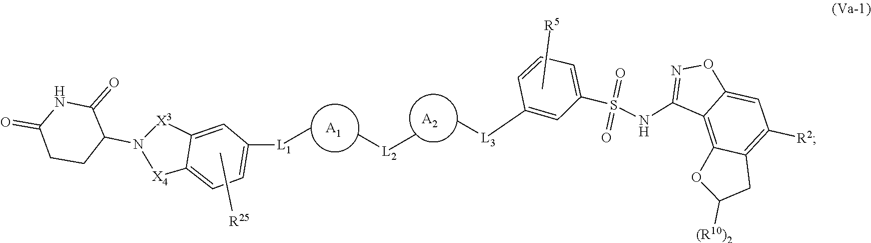

wherein
   ring A₁ and ring A₂ are each independently selected from:

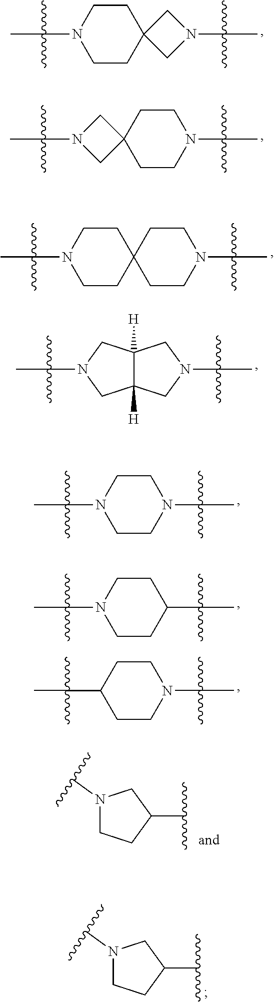

L₁ is a bond or (CH₂)ₚ;
L₂ is a bond or (CH₂)ₚ;
L₃ is a bond or (CH₂)ₚ;
p is 1, 2, 3 or 4;
each R¹⁰ is independently H or C₁-C₄ alkyl;
R² is —(CH₂)ₙ-(5-9 membered heteroaryl ring);
n=0 or 1;
X₃ is CH₂, CO, CH═CH (when X₄═CO), or N═CH (when X₄═CO); and
X₄ is CH₂, CO, CH═CH (when X₃═CO), or N═CH (when X₃═CO).

In some embodiments, at least one of ring A₁ and ring A₂ in Formula Va-1 is a 4-15-membered heterocycloalkyl group. In some embodiments, at least one of ring A₁ and ring A₂ Formula Va-1 is

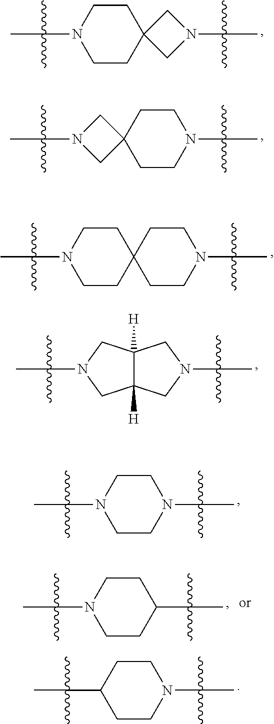

In some embodiments, at least one of ring A₁ and ring A₂ in Formula Va-1 is

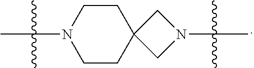 [SMILES: *N1CCC2(CC1)CN(*)C2]

In some embodiments, at least one of ring A₁ and ring A₂ in Formula Va-1 is

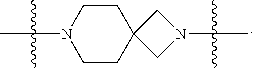 [SMILES: *N1CCC2(CC1)CN(*)C2]

In other embodiments, at least one of ring A₁ and ring A₂ in Formula Va-1 is

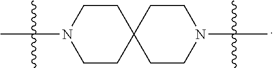 [SMILES: *N1CCC2(CC1)CCN(*)CC2]

In other embodiments, at least one of ring A₁ and ring A₂ in Formula Va-1 is

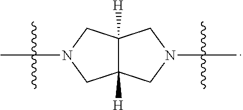 [SMILES: *N1C[C@H]2CN(*)C[C@@H]2C1]

In other embodiments, at least one of ring A₁ and ring A₂ in Formula Va-1 is

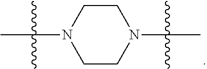 [SMILES: *N1CCN(*)CC1]

In yet other embodiments, at least one of ring A₁ and ring A₂ in Formula Va-1 is

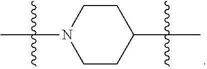 [SMILES: *C1CCN(*)CC1]

In yet other embodiments, at least one of ring A₁ and ring A₂ in Formula Va-1 is

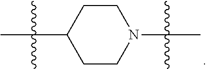 [SMILES: *C1CCN(*)CC1]

According to the disclosure, Y-PTM is a compound of Formula VIa or Formula VIb:

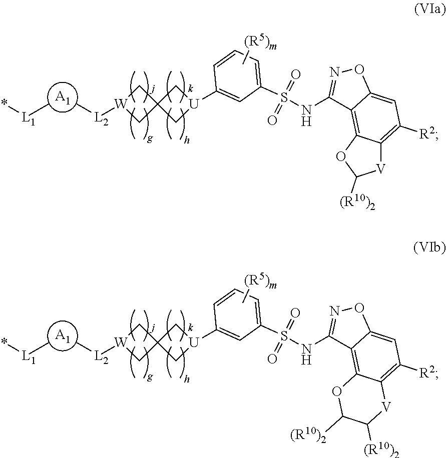

or a pharmaceutically acceptable salt thereof; wherein
   * is a point of attachment to ULM;
   L₁ is a bond, (C(R¹⁰)₂)ₚ, or CO;
   L₂ is a bond, (C(R¹⁰)₂)ₚ, or CO;
   L₃ is a bond, (C(R¹⁰)₂)ₚ, or CO;
   p is 1, 2, 3 or 4;
   each R¹⁰ is independently H or C₁-C₄ alkyl;
   ring A₁ is a 3-15 membered cycloalkyl group, a 4-15-membered heterocycloalkyl group, an aryl group, or a heteroaryl group;
   W is C(R¹⁵) or N;
   U is C(R¹⁵) or N;
   V is O or C(R¹⁰)₂;
   R¹⁵ is H, fluoro, C₁₋₄alkyl or C₁₋₄alkoxide;
   each g and h is independently 1, 2 or 3; and
   each j and k is independently 0, 1, 2 or 3.

In some embodiments, W is C(R¹⁵). In some embodiments, W is CH. In other embodiments, W is N. In some embodiments, U is CR¹⁵. In other embodiments, U is N. In some embodiments, both U and W are N.

In some embodiments, R¹⁵ is H. In some embodiments, R¹⁵ is halogen. In other embodiments, R¹⁵ is F. In some embodiments, R¹⁵ is C₁₋₆alkyl. In other embodiments, R¹⁵ is methyl or ethyl. In some embodiments, R¹⁵ is C₁₋₆alkoxide.

In some embodiments, g is 1, 2 or 3. In some embodiments, g is 1. In other embodiments, g is 2. In other embodiments, g is 3.

In some embodiments, h is 1, 2 or 3. In some embodiments, h is 1. In other embodiments, h is 2. In other embodiments, h is 3.

In some embodiments, j is 0, 1, 2 or 3. In some embodiments, j is 0. In some embodiments, j is 1. In other embodiments, j is 2. In other embodiments, j is 3.

In some embodiments, k is 0, 1, 2 or 3. In some embodiments, k is 0. In some embodiments, k is 1. In other embodiments, k is 2. In other embodiments, k is 3.

According to the disclosure, ULM-Y-PTM is a compound of Formula VIIa or Formula VIIb:

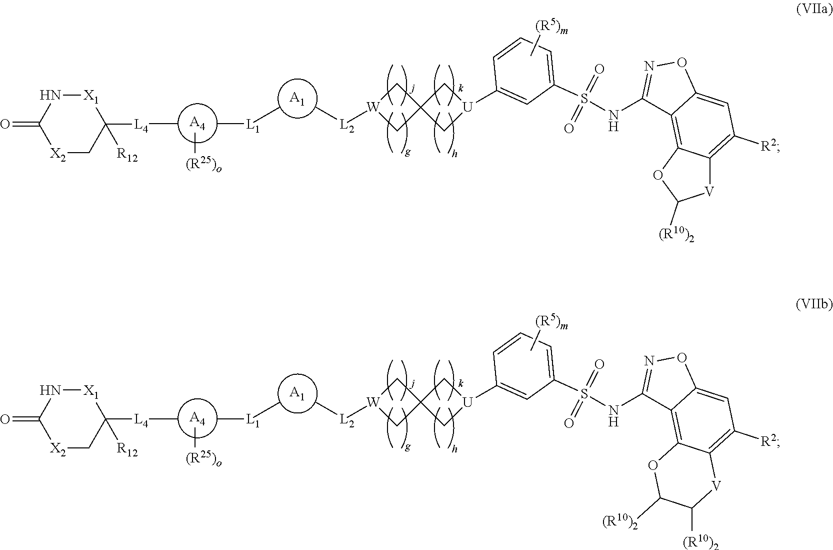

or a pharmaceutically acceptable salt thereof; wherein
   L₁ is a bond, (C(R¹⁰)₂)ₚ, or CO;
   L₂ is a bond, (C(R¹⁰)₂)ₚ, or CO;
   p is 1, 2, 3 or 4;
   each R¹⁰ is independently H or C₁-C₄ alkyl;
   W is C(R¹⁵) or N;
   U is C(R¹⁵) or N;
   V is O or C(R¹⁰)₂;
   R¹⁵ is H, fluoro, C₁₋₄alkyl or C₁₋₄alkoxide;
   each g and h is independently 1, 2 or 3;
   each j and k is independently 0, 1, 2 or 3;
   ring A₁ is a 3-7 membered cycloalkyl group, a 4-10-membered heterocycloalkyl group, an aryl group, or a heteroaryl group;
   Ring A₄ is a monocyclic, bicyclic or tricyclic aryl, heteroaryl or heterocycle group;
   L₄ is a bond, —O—, —S—, —NR^{a}—, —C(R^{a})₂— —C(O)NR^{a}—;
   X₁ is CH₂, CO, CH═CH (when X₂═CO), or N═CH (when X₂═CO);
   X₂ is CH₂, CO, CH═CH (when X₁═CO), or N═CH (when X₁═CO);
   R¹² is H, D, optionally substituted C₁₋₄ alkyl, C₁₋₄ alkoxyl, C₁₋₄haloalkyl, —CN, —OR^{a}, —OR^{b} or —SR^{b};
   each R²⁵ is independently H, halogen, oxo, —OH, —CN, —NO₂, —C₁-C₆alkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{d})(OR^{c}) or —S(O)₂R^{b};
   each R^{a} is independently H, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)R^{c}R^{b}, —P(O)OR^{c}OR^{b}, —S(O)R^{b}, —S(O)NR^{c}R^{d}, —S(O)₂R^{b}, —S(O)₂NR—R^{d}, SiR^{b}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl;
   each R^{b}, is independently H, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl;
   each R^{c} or R^{d} is independently H, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; or
   R^{c} and R^{d}, together with the atom to which they are both attached, form a monocyclic or multicyclic heterocycloalkyl, or a monocyclic or multicyclic heterocyclo-alkenyl group; and
   is 1, 2, 3, 4, or 5.

According to the disclosure, ULM-Y-PTM is a compound of Formula Villa or Formula VIIIb:

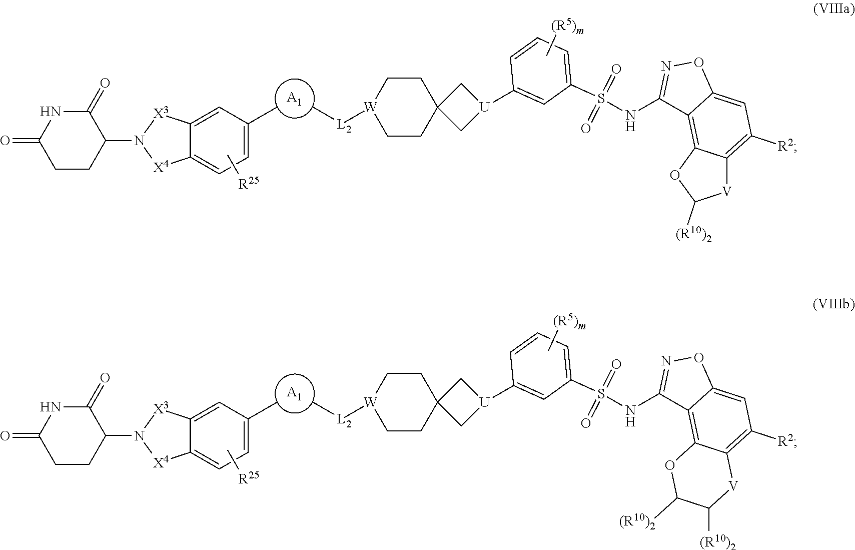

or a pharmaceutically acceptable salt thereof; wherein
   X₃ is CH₂, CO, CH═CH (when X₄═CO), or N═CH (when X₄═CO);
   X₄ is CH₂, CO, CH═CH (when X₃═CO), or N═CH (when X₃═CO); and
wherein V, W, U, L₂, ring A₁, R², R⁵, R¹⁰, R²⁵ and m are as defined herein.

According to the disclosure, ULM-Y-PTM is a compound of Formula IXa or Formula IXb:

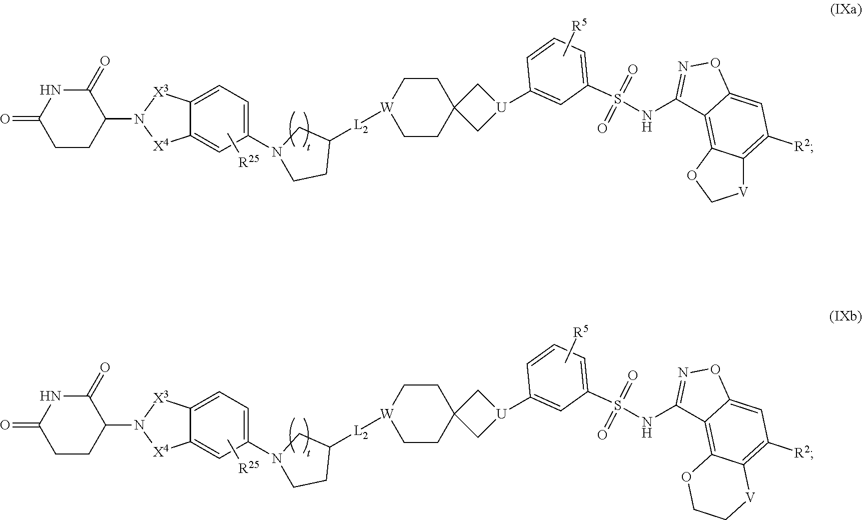

or a pharmaceutically acceptable salt thereof; wherein
   t is 1 or 2; and
   wherein X₃, X₄, V, W, U, L₂, R², R⁵, and R²⁵ are as defined herein.

In some embodiments, t is 1. In other embodiments, t is 2.

According to the disclosure, ULM-Y-PTM is a compound of Formula Xa:

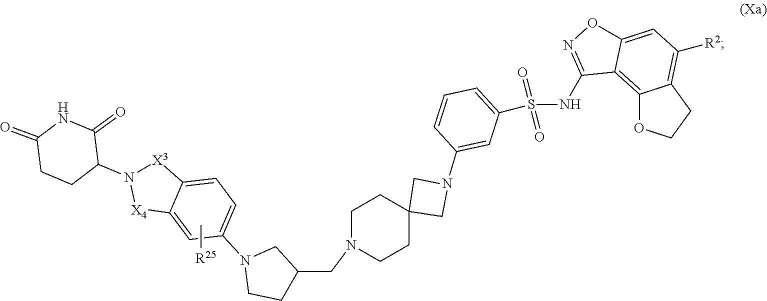

or a pharmaceutically acceptable salt thereof; wherein X₃, X₄, R² and R²⁵ are as defined herein.

In some embodiments, the compounds are:
N-(4-((1H-Pyrazol-1-yl)methyl)-2,3-dihydrobenzofuro[7,6-d]isoxazol-8-yl)-3-(7-(((S)-1-(2-((S)-2,6-dioxopiperidin-3-yl)-1-oxoisoindolin-5-yl)pyrrolidin-3-yl)methyl)-2,7-diazaspiro [3.5]nonan-2-yl)benzenesulfonamide;
N-(4-((1H-Pyrazol-1-yl)methyl)-2,3-dihydrobenzofuro[7,6-d]isoxazol-8-yl)-3-((3aS,6aS)-5-(((S)-1-(2-((S)-2,6-dioxopiperidin-3-yl)-1-oxoisoindolin-5-yl)pyrrolidin-3-yl)methyl)hexahydropyrrolo[3,4-c]pyrrol-2(1H)-yl)benzenesulfonamide;
N-(4-((1H-Pyrazol-1-yl)methyl)-2,3-dihydrobenzofuro[7,6-d]isoxazol-8-yl)-3-(9-(((S)-1-(2-((S)-2,6-dioxopiperidin-3-yl)-1-oxoisoindolin-5-yl)pyrrolidin-3-yl)methyl)-3,9-diazaspiro [5.5]undecan-3-yl)benzenesulfonamide;
N-(4-((1H-Pyrazol-1-yl)methyl)-2,3-dihydrobenzofuro[7,6-d]isoxazol-8-yl)-3-(2-(((S)-1-(2-((S)-2,6-dioxopiperidin-3-yl)-1-oxoisoindolin-5-yl)pyrrolidin-3-yl)methyl)-2,7-diazaspiro [3.5]nonan-7-yl)benzenesulfonamide;
N-(4-((1H-Pyrazol-1-yl)methyl)-2,3-dihydrobenzofuro[7,6-d]isoxazol-8-yl)-3-(4-((((S)-1-(2-((S)-2,6-dioxopiperidin-3-yl)-1-oxoisoindolin-5-yl)pyrrolidin-3-yl)methyl)amino) piperidin-1-yl)benzenesulfonamide;
N-(4-((1H-Pyrazol-1-yl)methyl)-2,3-dihydrobenzofuro[7,6-d]isoxazol-8-yl)-3-(4-(7-(2-(2,6-dioxopiperidin-3-yl)-1,3-dioxoisoindolin-5-yl)-7-azaspiro[3.5]nonan-2-yl)piperazin-1-yl)benzenesulfonamide;
N-(4-((1H-Pyrazol-1-yl)methyl)-2,3-dihydrobenzofuro[7,6-d]isoxazol-8-yl)-3-(4-((1-(2-(2,6-dioxopiperidin-3-yl)-1,3-dioxoisoindolin-4-yl)piperidin-4-yl)methyl)piperazin-1-yl)benzenesulfonamide;
N-(4-((1H-Pyrazol-1-yl)methyl)-2,3-dihydrobenzofuro[7,6-d]isoxazol-8-yl)-3-(4-((1-((1-(2-(2,6-dioxopiperidin-3-yl)-1,3-dioxoisoindolin-5-yl)pyrrolidin-3-yl)methyl)piperidin-4-yl)methyl)piperazin-1-yl)benzenesulfonamide;
N-(4-((1H-Pyrazol-1-yl)methyl)-2,3-dihydrobenzofuro[7,6-d]isoxazol-8-yl)-3-(4-((1-((1-(2-(2,6-dioxopiperidin-3-yl)-1,3-dioxoisoindolin-5-yl)piperidin-4-yl)methyl)piperidin-4-yl)methyl)piperazin-1-yl)benzenesulfonamide;
   or a pharmaceutically acceptable salt thereof.

In some embodiments, the compounds are:
N-(4-((1H-Pyrazol-1-yl)methyl)-2,3-dihydrobenzofuro[7,6-d]isoxazol-8-yl)-3-(7-(((S)-1-(2-((S)-2,6-dioxopiperidin-3-yl)-1-oxoisoindolin-5-yl)pyrrolidin-3-yl)methyl)-2,7-diazaspiro[3.5]nonan-2-yl)-2,6-dimethoxybenzenesulfonamide;
N-(4-((1H-Pyrazol-1-yl)methyl)-2,3-dihydrobenzofuro[7,6-d]isoxazol-8-yl)-3-(7-((1-(2-(2,6-dioxopiperidin-3-yl)-1,3-dioxoisoindolin-5-yl)piperidin-4-yl)methyl)-2,7-diazaspiro[3.5]nonan-2-yl)-2,6-dimethoxybenzenesulfonamide;
N-(4-((1H-Pyrazol-1-yl)methyl)-2,3-dihydrobenzofuro[7,6-d]isoxazol-8-yl)-3-(7-((1-(2-(2,6-dioxopiperidin-3-yl)-1,3-dioxoisoindolin-5-yl)piperidin-4-yl)methyl)-2,7-diazaspiro[3.5]nonan-2-yl)-2-methoxybenzenesulfonamide;
N-(4-((1H-Pyrazol-1-yl)methyl)-2,3-dihydrobenzofuro[7,6-d]isoxazol-8-yl)-3-(7-(((S)-1-(2-((S)-2,6-dioxopiperidin-3-yl)-1-oxoisoindolin-5-yl)pyrrolidin-3-yl)methyl)-2,7-diazaspiro[3.5]nonan-2-yl)-2-methoxybenzenesulfonamide;
N-(4-((1H-Pyrazol-1-yl)methyl)-2,3-dihydrobenzofuro[7,6-d]isoxazol-8-yl)-3-(7-((1-(2-(2,6-dioxopiperidin-3-yl)-1,3-dioxoisoindolin-4-yl)piperidin-4-yl)methyl)-2,7-diazaspiro[3.5]nonan-2-yl)-2-methoxybenzenesulfonamide;
N-(4-((1H-Pyrazol-1-yl)methyl)-2,3-dihydrobenzofuro[7,6-d]isoxazol-8-yl)-4-(7-(((S)-1-(2-((S)-2,6-dioxopiperidin-3-yl)-1-oxoisoindolin-5-yl)pyrrolidin-3-yl)methyl)-2,7-diazaspiro[3.5]nonan-2-yl)benzenesulfonamide;
   or a pharmaceutically acceptable salt thereof.

It will be apparent that the compounds of the invention, including all subgenera described herein, may have multiple stereogenic centers. As a result, there exist multiple stereoisomers (enantiomers and diastereomers) of the compounds (and subgenera described herein). The present disclosure contemplates and encompasses each stereoisomer of any compound of encompassed by the disclosure as well as mixtures of said stereoisomers.

Pharmaceutically acceptable salts and solvates of the compounds of the disclosure (including all subgenera described herein) are also within the scope of the disclosure.

Isotopic variants of the compounds of the disclosure (including all subgenera described herein) are also contemplated by the present disclosure. Isotopes include those atoms have the same atomic number but different mass numbers. For example, isotopes of hydrogen are tritium and deuterium.

### Pharmaceutical Compositions and Methods of Administration

The subject pharmaceutical compositions are typically formulated to provide a therapeutically effective amount of a compound of the present disclosure as the active ingredient, or a pharmaceutically acceptable salt, ester, prodrug, solvate, hydrate or derivative thereof. Where desired, the pharmaceutical compositions contain pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, the one or more compounds of the invention and other agent(s) may be mixed into a preparation or both components may be formulated into separate preparations to use them in combination separately or at the same time.

In some embodiments, the concentration of one or more compounds provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v or v/v.

In some embodiments, the concentration of one or more compounds of the invention is greater than 90%, 80%, 70%, 60%, 50%, 40%, 30%, 20%, 19.75%, 19.50%, 19.25%, 19%, 18.75%, 18.50%, 18.25% 18%, 17.75%, 17.50%, 17.25% 17%, 16.75%, 16.50%, 16.25%, 16%, 15.75%, 15.50%, 15.25% 15%, 14.75%, 14.50%, 14.25% 14%, 13.75%, 13.50%, 13.25%, 13%, 12.75%, 12.50%, 12.25%, 12%, 11.75%, 11.50%, 11.25% 11%, 10.75%, 10.50%, 10.25% 10%, 9.75%, 9.50%, 9.25%, 9%, 8.75%, 8.50%, 8.25% 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v, or v/v.

In some embodiments, the concentration of one or more compounds of the invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds of the invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g (or a number in the range defined by and including any two numbers above).

In some embodiments, the amount of one or more compounds of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g (or a number in the range defined by and including any two numbers above).

In some embodiments, the amount of one or more compounds of the invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

The compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the route of administration, the form in which the compound is administered, the subject to be treated, the body weight of the subject to be treated, and the preference and experience of the attending physician.

A pharmaceutical composition of the invention typically contains an active ingredient (e.g., a compound of the disclosure) of the present invention or a pharmaceutically acceptable salt and/or coordination complex thereof, and one or more pharmaceutically acceptable excipients, carriers, including but not limited to inert solid diluents and fillers, diluents, sterile aqueous solution and various organic solvents, permeation enhancers, solubilizers and adjuvants.

Described below are non-limiting exemplary pharmaceutical compositions and methods for preparing the same.

### Pharmaceutical Compositions for Oral Administration.

In some embodiments, the invention provides a pharmaceutical composition for oral administration containing a compound of the invention, and a pharmaceutical excipient suitable for oral administration.

In some embodiments, the invention provides a solid pharmaceutical composition for oral administration containing: (i) an effective amount of a compound of the invention; optionally (ii) an effective amount of a second agent; and (iii) a pharmaceutical excipient suitable for oral administration. In some embodiments, the composition further contains: (iv) an effective amount of a third agent.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (e.g., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidyl-choline, phosphatidylethanolamine, phosphatidylglycerol, phosphatidic acid, phosphatidyl-serine, lysophosphatidylcholine, lysophosphatidylethanolamine, lysophosphatidylglycerol, lysophosphatidic acid, lysophosphatidylserine, PEG-phosphatidylethanolamine, PVP -phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG- 15 oleate, PEG-20 oleate, PEG-20 dioleate, PEG-32 oleate, PEG-200 oleate, PEG-400 oleate, PEG- 15 stearate, PEG-32 distearate, PEG-40 stearate, PEG- 100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG- 100 succinate, PEG-24 cholesterol, polyglyceryl-lOoleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25% o, 50%), 100% o, or up to about 200%> by weight, based on the combined weight of the drug, and other excipients. If desired, very small amounts of solubilizer may also be used, such as 5%>, 2%>, 1%) or even less. Typically, the solubilizer may be present in an amount of about 1%> to about 100%, more typically about 5%> to about 25%> by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Injection.

In some embodiments, the invention provides a pharmaceutical composition for injection containing a compound of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical (e.g., Transdermal) Delivery.

In some embodiments, the invention provides a pharmaceutical composition for transdermal delivery containing a compound of the present invention and a pharmaceutical excipient suitable for transdermal delivery.

Compositions of the present invention can be formulated into preparations in solid, semisolid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation.

Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of a compound of the present invention in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

### Pharmaceutical Compositions for Inhalation.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

### Other Pharmaceutical Compositions.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the compounds or pharmaceutical composition of the present invention can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g. transdermal application), rectal administration, via local delivery by catheter or stent or through inhalation. Compounds can also be administered intraadiposally or intrathecally.

In some embodiments, the compounds or pharmaceutical composition of the present invention are administered by intravenous injection.

The amount of the compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g. by dividing such larger doses into several small doses for administration throughout the day.

In some embodiments, a compound of the invention is administered in a single dose.

Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of the invention may continue as long as necessary. In some embodiments, a compound of the invention is administered for more than 1, 2, 3, 4, 5, 6, 7, 14, or 28 days. In some embodiments, a compound of the invention is administered for less than 28, 14, 7, 6, 5, 4, 3, 2, or 1 day. In some embodiments, a compound of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Compounds of the invention may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. Compounds of the invention may additionally be administered intravascularly from a balloon used during angioplasty. Extravascular administration of the compounds via the pericard or via advential application of formulations of the invention may also be performed to decrease restenosis.

A variety of stent devices which may be used as described are disclosed, for example, in the following references, all of which are hereby incorporated by reference: U.S. Pat. Nos. 5,451,233; 5,040,548; 5,061,273; 5,496,346; 5,292,331; 5,674,278; 3,657,744; 4,739,762; 5,195,984; 5,292,331; 5,674,278; 5,879,382; 6,344,053.

The compounds of the invention may be administered in dosages. It is known in the art that due to intersubject variability in compound pharmacokinetics, individualization of dosing regimen is necessary for optimal therapy. Dosing for a compound of the invention may be found by routine experimentation in light of the instant disclosure.

When a compound of the invention is administered in a composition that comprises one or more agents, and the agent has a shorter half-life than the compound of the invention unit dose forms of the agent and the compound of the invention may be adjusted accordingly.

The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.
Exemplary parenteral administration forms include solutions or suspensions of active compound in sterile aqueous solutions, for example, aqueous propylene glycol or dextrose solutions. Such dosage forms can be suitably buffered, if desired.

### Methods of Use

The method typically comprises administering to a subject a therapeutically effective amount of a compound of the invention. The therapeutically effective amount of the subject combination of compounds may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or down-regulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

In certain embodiment, the present invention provides a pharmaceutical composition comprising a compound of bispecific formula, or pharmaceutically acceptable salt thereof.

In certain embodiment, the present invention provides a pharmaceutical composition comprising a compound of bispecific formula for use in degrading a target protein in a cell.

In certain embodiment, a method of degrading a target protein comprising administering to a cell therapeutically effective amount of a bispecific compound, or pharmaceutically acceptable salt, wherein the compound is effective for degrading the target protein.

“Abnormal cell growth” or “cancer” as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (3) any tumors that proliferate by receptor tyrosine kinases; (4) any tumors that proliferate by aberrant serine/threonine kinase activation; (5) benign and malignant cells of other proliferative diseases in which aberrant serine/threonine kinase activation occurs; (6) any tumors that proliferate by aberrant signaling, metabolic, epigenetic and transcriptional mechanism; and (7) benign and malignant cells of other proliferative diseases in which aberrant signaling, metabolic, epigenetic and transcriptional mechanism.

For convenience, certain well-known abbreviations, may be used herein, including: estrogen receptor positive (ER+), human epidermal growth factor receptor 2 negative (HER2-), non-small cell lung cancer (NSCLC) and castration resistant prostate cancer (CRPC).

Further embodiments relate to methods of treating abnormal cell growth in a patient. Additional embodiments relate to a method of treating abnormal cell growth in a patient comprising administering to the patient an amount of a compound described herein that is effective in treating abnormal cell growth.

In other embodiments, the abnormal cell growth is cancer.

In some embodiments, the cancer is selected from the group consisting of lung cancer, mesothelioma, bone cancer, pancreatic cancer, skin cancer, cancer of the head or neck, cutaneous or intraocular melanoma, uterine cancer, ovarian cancer, rectal cancer, cancer of the anal region, stomach cancer, hepatic carcinoma, colon cancer, breast cancer, uterine cancer, carcinoma of the fallopian tubes, carcinoma of the endometrium, carcinoma of the cervix, carcinoma of the vagina, carcinoma of the vulva, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, hematology malignancy, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, glioblastoma, brain stem glioma, pituitary adenoma, or a combination of two or more of the foregoing cancers.

Additional embodiments relate to methods of treating solid tumors in a patient. Some embodiments relate to the treatment of solid tumors in a patient comprising administering to the patient an amount of a compound described herein that is effective in treating the solid tumor.

In one embodiment, the solid tumor is breast, lung, colon, brain, prostate, stomach, pancreatic, ovarian, melanoma, endocrine, uterine, testicular, or bladder.

In one embodiment, the solid tumor is breast, lung, prostate, pancreatic, or ovarian.

In one embodiment, the cancer is breast cancer.

In one embodiment, the breast cancer is ER+ breast cancer.

In one embodiment, the breast cancer is ER+ HER2− breast cancer.

In one embodiment, the breast cancer is locally advanced or metastatic ER+ HER2− breast cancer.

In one embodiment, the lung cancer is non-small cell lung cancer.

In one embodiment, the lung cancer is locally advanced or metastatic non-small cell lung cancer.

In one embodiment, the prostate cancer is castration resistant prostate cancer. In one embodiment, the prostate cancer is locally advanced or metastatic castration resistant prostate cancer.

Additional embodiments relate to methods of treating hematologic tumors in a patient. Some embodiments relate to the treatment of hematologic tumors in a patient comprising administering to the patient an amount of a compound described herein that is effective in treating the hematologic tumor.

In one embodiment, the hematologic tumor is leukemia, lymphoma or multiple myeloma.

In one embodiment, the hematologic tumor is leukemia or lymphoma.

Additional embodiments relate to methods of treating cancer in a patient comprising administering to the patient an amount of a compound described herein that is effective in treating cancer. In one embodiment, the cancer is breast, lung, colon, brain, prostate, stomach, pancreatic, ovarian, melanoma, endocrine, uterine, testicular, bladder, or hematologic.

In one embodiment, the cancer is breast, lung, prostate, pancreatic, ovarian, or hematologic.

In one embodiment, the cancer is breast, lung, prostate, pancreatic, or ovarian. In one embodiment, the cancer is breast cancer.

In one embodiment, the breast cancer is ER+ breast cancer.

In one embodiment, the breast cancer is ER+ HER2− breast cancer.

In one embodiment, the breast cancer is locally advanced or metastatic ER+ HER2− breast cancer.

In one embodiment, the lung cancer is non-small cell lung cancer.

In one embodiment, the lung cancer is locally advanced or metastatic non-small cell lung cancer.

In one embodiment, the prostate cancer is castration resistant prostate cancer. In one embodiment, the prostate cancer is locally advanced or metastatic castration resistant prostate cancer.

In one embodiment, the cancer is hematologic.

In one embodiment, the hematologic tumor is leukemia or lymphoma.

Further embodiments relate to methods of treating cancer in a patient which comprises administering to the patient an amount of a compound described herein that is effective in treating cancer in combination with an anti-tumor agent selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, and anti-androgens.

More embodiments relate to pharmaceutical compositions for treating cancer in a patient comprising an amount of a compound described herein that is effective in treating cancer, and a pharmaceutically acceptable carrier.

Additional embodiments relate to a method of treating cancer in a patient, and in particular a human, comprising administering to the patient an amount of a compound described herein, or a pharmaceutically acceptable salt, solvate, hydrate or prodrug thereof, that is effective in treating cancer. In one embodiment of this method, the cancer, includes, but is not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers.

In one embodiment the method comprises comprising administering to a patient an amount of a compound described herein that is effective in treating said cancer solid tumor. In one preferred embodiment the solid tumor is breast, lung, colon, brain, prostate, stomach, pancreatic, ovarian, skin (melanoma), endocrine, uterine, testicular, and bladder cancer.

In another embodiment of said method, said cancer is a benign proliferative disease, including, but not limited to, psoriasis, benign prostatic hypertrophy or restenosis.

Some embodiments relate to a method of treating cancer in a patient which comprises administering to said patient an amount of a compound described herein, or a pharmaceutically acceptable salt, solvate, hydrate or prodrug thereof, that is effective in treating cancer in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

Additional embodiments relate to a pharmaceutical composition for treating cancer in a patient, and in particular a human, comprising an amount of a compound described herein, or a pharmaceutically acceptable salt, solvate, hydrate or prodrug thereof, that is effective in treating cancer, and a pharmaceutically acceptable carrier. In one embodiment of said composition, the cancer, includes, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said pharmaceutical composition, said abnormal cell growth is a benign proliferative disease, including, but not limited to, psoriasis, benign prostatic hypertrophy or restinosis.

Further embodiments relate to a method of treating cancer in a patient which comprises administering to said patient an amount of a compound described herein, or a pharmaceutically acceptable salt, solvate, or hydrate thereof, that is effective in treating cancer in combination with another anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens. Some embodiments contemplate a pharmaceutical composition for treating abnormal cell growth wherein the composition includes a compound described herein, or a pharmaceutically acceptable salt, solvate, or hydrate thereof, that is effective in treating abnormal cell growth, and another anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

Yet more embodiments relate to a method of treating a disorder associated with angiogenesis in a patient, including a human, comprising administering to said patient an amount of a compound described herein, as defined above, or a pharmaceutically acceptable salt, solvate, hydrate or prodrug thereof, that is effective in treating said disorder in combination with one or more anti-tumor agents listed above. Such disorders include cancerous tumors such as melanoma; ocular disorders such as age-related macular degeneration, presumed ocular histoplasmosis syndrome, and retinal neovascularization from proliferative diabetic retinopathy; rheumatoid arthritis; bone loss disorders such as osteoporosis, Paget's disease, humoral hypercalcemia of malignancy, hypercalcemia from tumors metastatic to bone, and osteoporosis induced by glucocorticoid treatment; coronary restenosis; and certain microbial infections including those associated with microbial pathogens selected from adenovirus, hantaviruses, Borrelia burgdorferi, Yersinia spp., Bordetella pertussis, and group A Streptococcus.

Compounds of the disclosure, as well as pharmaceutical compositions comprising them, can be administered to treat any of the described diseases, alone or in combination with a medical therapy. Medical therapies include, for example, surgery and radiotherapy (e.g., gamma-radiation, neutron beam radiotherapy, electron beam radiotherapy, proton therapy, brachytherapy, systemic radioactive isotopes).

In other aspects, compounds of the disclosure, as well as pharmaceutical compositions comprising them, can be administered to treat any of the described diseases, alone or in combination with one or more other agents.

In other methods, the compounds of the disclosure, as well as pharmaceutical compositions comprising them, can be administered in combination with agonists of nuclear receptors agents.

In other methods, the compounds of the disclosure, as well as pharmaceutical compositions comprising them, can be administered in combination with antagonists of nuclear receptors agents.

In other methods, the compounds of the disclosure, as well as pharmaceutical compositions comprising them, can be administered in combination with an anti-proliferative agent.

### Combination Therapies

For treating cancer and other proliferative diseases, the compounds of the invention can be used in combination with chemotherapeutic agents, agonists or antagonists of nuclear receptors, or other anti-proliferative agents. The compounds of the invention can also be used in combination with a medical therapy such as surgery or radiotherapy, e.g., gamma-radiation, neutron beam radiotherapy, electron beam radiotherapy, proton therapy, brachytherapy, and systemic radioactive isotopes. Examples of suitable chemotherapeutic agents include any of: abarelix, aldesleukin, alemtuzumab, alitretinoin, allopurinol, all-trans retinoic acid, altretamine, anastrozole, arsenic trioxide, asparaginase, azacitidine, bendamustine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panobinostat, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinstat and zoledronate.

Some embodiments relate to a method of (and to a pharmaceutical composition for) treating cancer in a patient which comprise an amount of a compound described herein, or a pharmaceutically acceptable salt, solvate, or hydrate thereof, in combination with an amount of one or more substances selected from anti-angiogenesis agents, signal transduction inhibitors (e.g., inhibiting the means by which regulatory molecules that govern the fundamental processes of cell growth, differentiation, and survival communicated within the cell), and antiproliferative agents, which amounts are together effective in treating said abnormal cell growth.

Anti-angiogenesis agents, such as MMP-2 (matrix-metalloprotienase 2) inhibitors, MMP-9 (matrix-metalloprotienase 9) inhibitors, and COX-II (cyclooxygenase II) inhibitors, can be used in conjunction with a compound described herein in the methods and pharmaceutical compositions described herein.

Tyrosine kinase inhibitors can also be combined with a compound described herein.

VEGF inhibitors, for example, sutent and axitinib, can also be combined with a compound described herein.

ErbB2 receptor inhibitors may be administered in combination with a compound described herein. Various other compounds, such as styrene derivatives, have also been shown to possess tyrosine kinase inhibitory properties, and some of tyrosine kinase inhibitors have been identified as erbB2 receptor inhibitors.

Epidermal growth factor receptor (EGFR) inhibitors may be administered in combination with a compound of the present invention. PI3K inhibitors, such as PI3K alpha or PI3K beta inhibitors, may be administered in combination with a compound of the present invention.

Mammalian target of rapamycin (mTOR) inhibitors may be administered in combination with a compound of the present invention.

c-Met inhibitors may be administered in combination with a compound of the present invention.

CDK inhibitors may be administered in combination with a compound of the present invention.

MEK inhibitors may be administered in combination with a compound of the present invention.

PARP inhibitors may be administered in combination with a compound of the present invention.

JAK inhibitors may be administered in combination with a compound of the present invention.

An antagonist of a Programmed Death 1 protein (PD-1) may be administered in combination with a compound of the present invention.

An antagonist of Programmed Death-Ligand 1 (PD-L1) may be administered in combination with a compound of the present invention.

Other antiproliferative agents that may be used with the compounds described herein include inhibitors of the enzyme farnesyl protein transferase and inhibitors of the receptor tyrosine kinase PDGFr.

A compound described herein may also be used with other agents useful in treating abnormal cell growth or cancer, including, but not limited to, agents capable of enhancing antitumor immune responses, such as CTLA4 (cytotoxic lymphocyte antigen 4) antibodies, and other agents capable of blocking CTLA4; and anti-proliferative agents such as other farnesyl protein transferase inhibitors, for example the farnesyl protein transferase.

A compound described herein may be applied as a sole therapy or may involve one or more other anti-tumor substances, for example those selected from, for example, mitotic inhibitors, alkylating agents, anti-metabolites, growth factor inhibitors, cell cycle inhibitors, intercalating antibiotics, enzymes, and anti-hormones.

The compounds described herein may be used alone or in combination with one or more of a variety of anti-cancer agents or supportive care agents. For example, the compounds described herein may be used with cytotoxic agents. Some embodiments also contemplate the use of the compounds described herein together with hormonal therapy. Further, some embodiments provide a compound described herein alone or in combination with one or more supportive care products, e.g., a product selected from the group consisting of Filgrastim (Neupogen), ondansetron (Zofran), Fragmin, Procrit, Aloxi, Emend, or combinations thereof. Such conjoint treatment may be achieved by way of the simultaneous, sequential or separate dosing of the individual components of the treatment.

The compounds described herein may be used with antitumor agents, alkylating agents, antimetabolites, antibiotics, plant-derived antitumor agents, camptothecin derivatives, tyrosine kinase inhibitors, antibodies, interferons, and/or biological response modifiers. In this regard, the following is a non-limiting list of examples of secondary agents that may be used with the compounds described herein.

Compounds of the invention can be prepared using numerous preparatory reactions known in the literature. The Schemes below provide general guidance in connection with preparing the compounds of the invention. One skilled in the art would understand that the preparations shown in the Schemes can be modified or optimized using general knowledge of organic chemistry to prepare various compounds of the invention. Example synthetic methods for preparing compounds of the invention are provided in the Schemes below.

The following Examples are provided to illustrate some of the concepts described within this disclosure. While the Examples are considered to provide an embodiment, it should not be considered to limit the more general embodiments described herein.

### EXAMPLES

### General Synthetic Procedures

The compounds of the Invention may be prepared using the general procedures described below. The compounds described herein may be prepared according to the following synthetic schemes and general synthetic procedures.

Compounds of Formula (I) can be synthesized using, for example, the sequences shown in Scheme I. Coupling of optionally protected compounds 1-1 where RG¹ is a reactive group such as, but not limited to, halogen (e.g., F, Cl, Br, I), hydroxy, aldehyde, boronic acid, boronate ester, trialkyltin, carboxylic acid, or amine with optionally protected compounds 1-2 where RG² and RG³ are each independently reactive groups such as, but not limited to, halogen (e.g., F, Cl, Br, I), hydroxy, aldehyde, boronic acid, boronate ester, trialkyltin, carboxylic acid, or amine using appropriate synthetic methods (such as, but not limited to, S_{N}Ar reaction, Suzuki coupling, Stille coupling, Buchwald-Hartwig reaction, Mitsunobu reaction, Williamson ether synthesis, amide coupling, or reductive amination) can afford compounds 1-3. Coupling of compounds 1-3 with optionally protected compounds 1-4 where RG⁴ is a reactive group such as halogen (e.g., F, Cl, Br, I), hydroxy, aldehyde, boronic acid, boronate ester, trialkyltin, carboxylic acid, or amine using appropriate synthetic methods (such as, but not limited to, S_{N}Ar reaction, Suzuki coupling, Stille coupling, Buchwald-Hartwig reaction, Mitsunobu reaction, Williamson ether synthesis, amide coupling, or reductive amination) can give compounds of Formula (I).

Alternatively, the synthesis can be achieved by the coupling of optionally protected compounds 1-4 with optionally protected compounds 1-2 to afford compounds 1-6 using appropriate synthetic methods mentioned above and subsequent reaction of 1-6 with optionally protected compounds 1-1 using appropriate synthetic methods mentioned above to afford compounds of Formula (I).

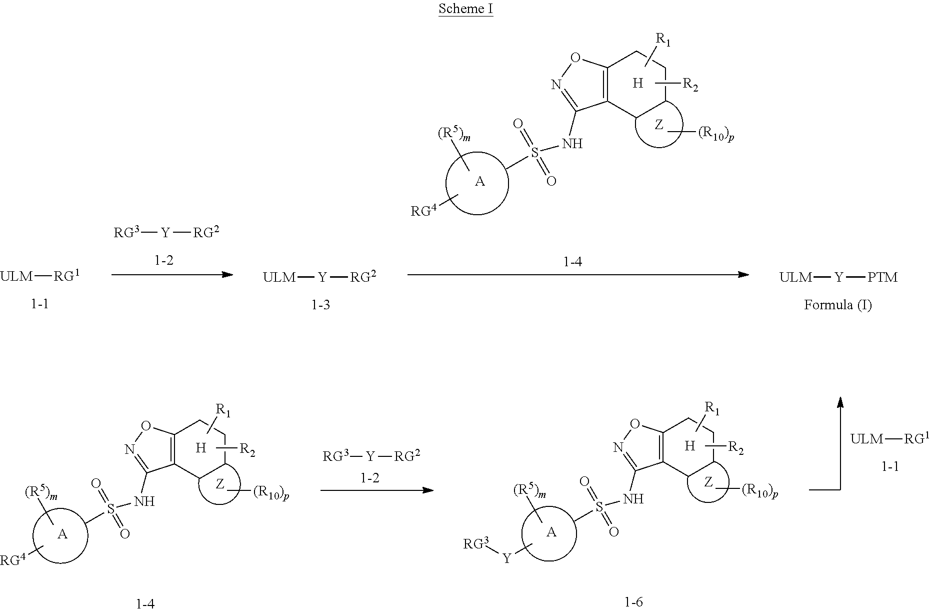

Intermediates for the synthesis of compounds of Formula (I) can be prepared as described in Scheme II. Amines 2-1 can be sulfonylated with sulfonyl halides 2-2 where X^{a} is a halogen (e.g., F, Cl, or Br) under standard sulfonylation conditions (e.g., in the presence of a base such as KOtBu, LiHMDS, or pyridine) to afford compounds 1-4.

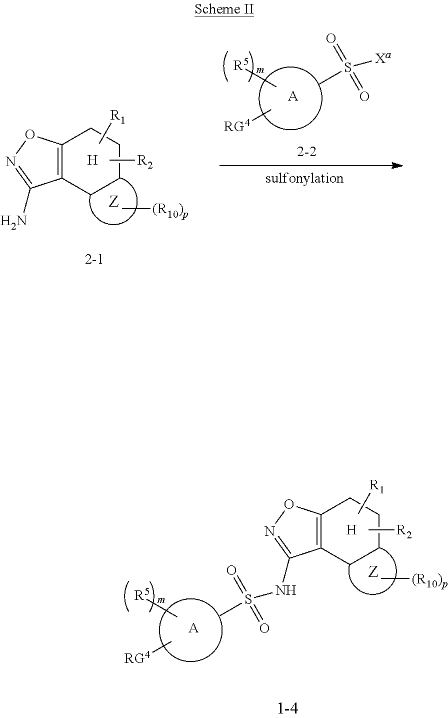

Compounds of Formula (I) can be prepared as described in Scheme III. Coupling of optionally protected compounds 1-1 with compounds 3-1 where RG⁵ and RG⁶ are each independently reactive groups such as, but not limited to, halogen (e.g., F, Cl, Br, I), hydroxy, aldehyde, boronic acid, boronate ester, trialkyltin, carboxylic acid, or amine and y is an integer from 0 to 14 using appropriate synthetic methods (such as, but not limited to, S_{N}Ar reaction, Suzuki coupling, Stille coupling, Buchwald-Hartwig reaction, Mitsunobu reaction, Williamson ether synthesis, amide coupling, or reductive amination) can afford compounds 3-2. Reaction of optionally protected 1-4 with compounds 3-3 where RG⁷ and RG⁸ are each independently reactive groups such as, but not limited to, halogen (e.g., F, Cl, Br, I), hydroxy, aldehyde, boronic acid, boronate ester, trialkyltin, carboxylic acid, or amine and z is an integer from 0 to 14 and the sum of y and z is an integer from 1 to 14 using appropriate synthetic methods (such as, but not limited to, S_{N}Ar reaction, Suzuki coupling, Stille coupling, Buchwald-Hartwig reaction, Mitsunobu reaction, Williamson ether synthesis, amide coupling, or reductive amination) can afford compounds 3-4. Coupling of compounds 3-2 and 3-4 using appropriate synthetic methods (such as, but not limited to, S_{N}Ar reaction, Suzuki coupling, Stille coupling, Buchwald-Hartwig reaction, Mitsunobu reaction, Williamson ether synthesis, amide coupling, or reductive amination) can afford compounds 3-5.

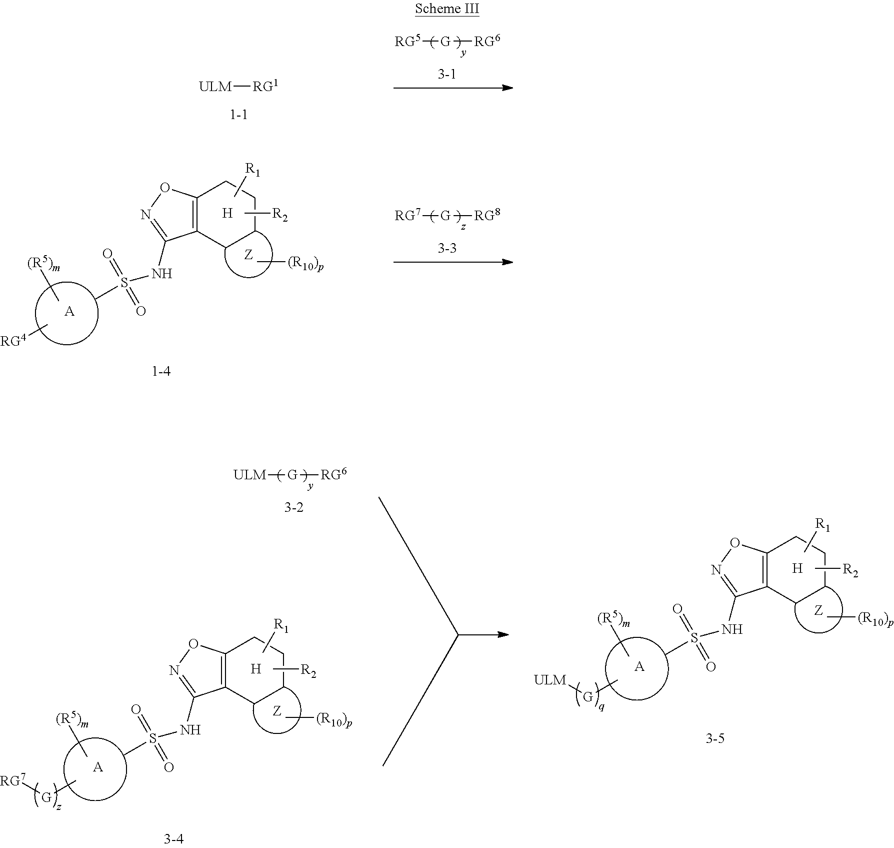

Intermediates for the preparation of compounds of Formula I can be synthesized according to the route described in Scheme IV. Reaction of thalidomides 4-1 where X^{b} is a halogen (e.g., F or Cl) or pseudohalogen (e.g., OTf or OMs) with compounds 4-2 where ring A₁ₐ is an optionally protected nitrogen-containing 3-11 membered heterocyclyl that is optionally substituted with a reactive group such as, but not limited to, halogen (e.g., F, Cl, Br, I), hydroxy, aldehyde, boronic acid, boronate ester, trialkyltin, carboxylic acid, or amine, under standard S_{N}Ar conditions optionally in the presence of a base (e.g., DIPEA) can afford compounds 4-3.

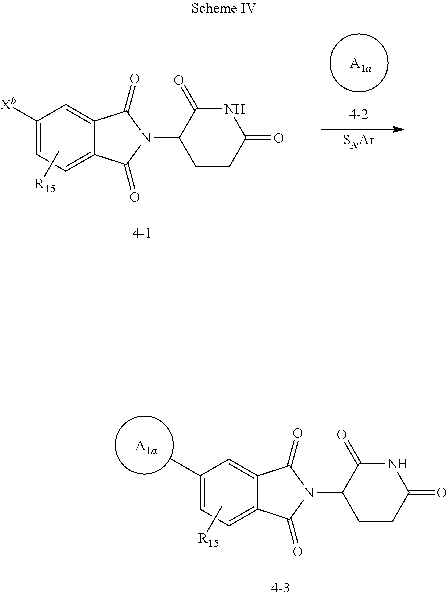

Intermediates for the preparation of compounds of Formula I can be synthesized according to the route described in Scheme V. Coupling of compounds 5-1 where X^{c} is a halogen (e.g., F, Cl, or Br) or pseudohalogen (e.g., OTf or OMs) and R^{z} is a C₁-C₄ alkyl group with compounds 4-2 under standard S_{N}Ar conditions optionally in the presence of a base (e.g., DIPEA) or under standard Buchwald-Hartwig amination conditions (e.g., in the presence of a palladium catalyst, such as XPhos Pd G₂ or BrettPhos Pd G₃, and a base, such as K₃PO₄ or sodium tert-butoxide) can afford compounds 5-2. Reduction of nitriles 5-2 under appropriate conditions (e.g., Raney Nickel) can yield aldehydes 5-3. Reductive amination and cyclization of compounds 5-3 with amine 5-4 under standard conditions such as addition of an appropriate acid (e.g., AcOH) and reducing agent (e.g., sodium triacetoxyborohydride) and then a base (e.g., DIPEA) can afford compounds 5-5.

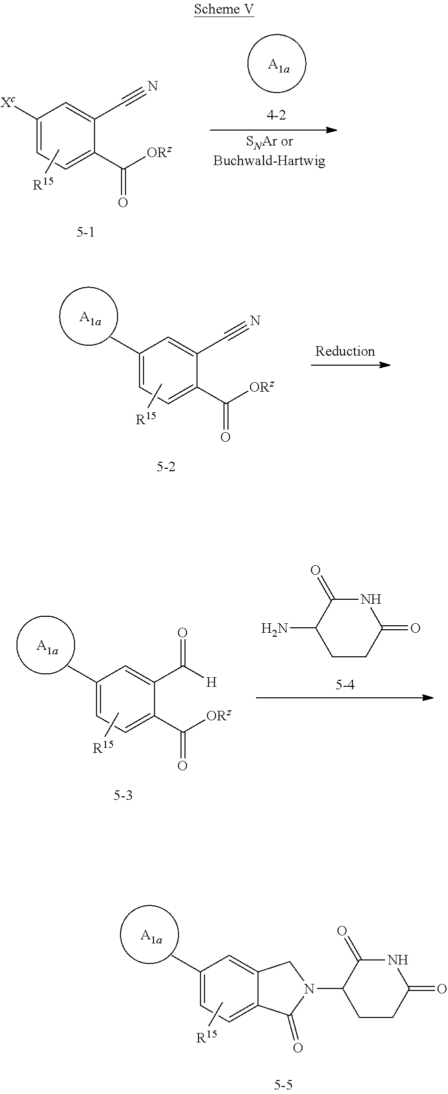

Intermediates for the preparation of compounds of Formula I can be synthesized according to the route described in Scheme VI. Coupling of compounds 6-1 where X^{d} is a halogen (e.g., F, Cl, or Br) or pseudohalogen (e.g., OTf or OMs) and Ry is a C₁-C₄ alkyl group with compounds 4-2 under standard S_{N}Ar conditions optionally in the presence of a base (e.g., DIPEA) or under standard Buchwald-Hartwig amination conditions (e.g., in the presence of a palladium catalyst, such as XPhos Pd G2 or BrettPhos Pd G3, and a base, such as K₃PO₄ or sodium tert-butoxide) can afford compounds 6-2. Reduction of nitriles 6-2 under appropriate conditions (e.g., Raney Nickel) can yield aldehydes 6-3. Reductive amination and cyclization of compounds 6-3 with amine 5-4 under standard conditions such as addition of an appropriate acid (e.g., AcOH) and reducing agent (e.g., sodium triacetoxyborohydride) and then a base (e.g., DIPEA) can afford compounds 6-4.

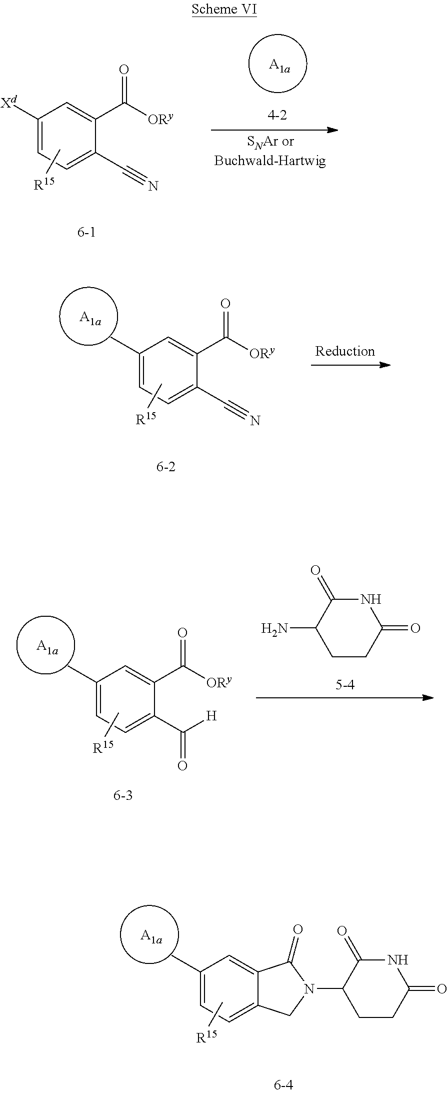

Compounds of Formula (I) can be prepared as described in Scheme VII. Oxidation of alcohols 7-1 where R^{X} is H or C₁-C₄ alkyl under standard conditions such as in the presence of an oxidant (e.g., Dess-Martin periodinane, 2-iodoxybenzoic acid, or SO₃·pyridine) and optionally in the presence of a base (e.g., NaHCO₃ or Et₃N) can afford compounds 7-2. Coupling of compounds 7-3 where X^{e} is a halogen (e.g., F, Cl, Br, or I) or pseudohalogen (e.g., OMs or OTf) with compounds 7-4 where A₂ₐ is 3-11 membered diazaheterocyclyl and P¹ is an appropriate nitrogen protecting group (e.g., Boc, Cbz, Bn, PMB, or acetyl) under standard S_{N}Ar conditions optionally in the presence of a base (e.g., DIPEA) or under standard Buchwald-Hartwig amination conditions (e.g., in the presence of a palladium catalyst, such as BrettPhos Pd G₃, and a base, such as sodium tert-butoxide) and subsequent deprotection can afford compounds 7-5. Reaction of compounds 7-2 and amines 7-5 under standard conditions for reductive amination (e.g., in the presence of a reducing agent such as sodium triacetoxy-borohydride or sodium cyanoborohydride and optionally an acid, such as acetic acid) can afford compounds 7-6.

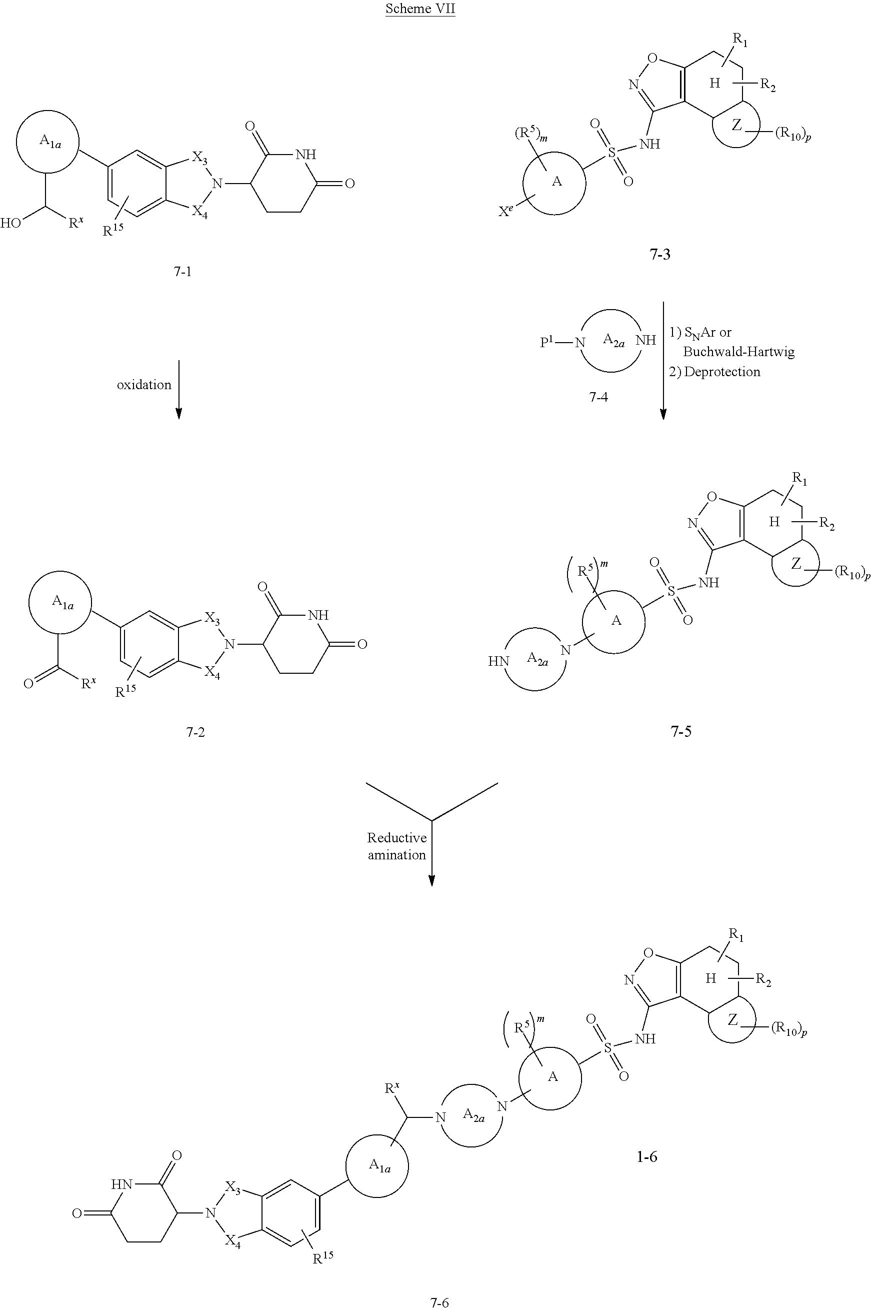

Intermediates for the preparation of compounds of Formula I can be synthesized according to the route described in Scheme VIII. Compounds 5-3 can be converted to compounds 8-2 through reductive amination and cyclization with compound 8-1 that is optionally enantioenriched under standard conditions such as in the presence of an appropriate acid (e.g., acetic acid) and a reducing agent (e.g., sodium triacetoxyborohydride) and then a base (e.g., N,N-diisopropylethylamine). Cyclization of 8-2 in the presence of a base (e.g., potassium tert-butoxide) can afford compounds 5-5.

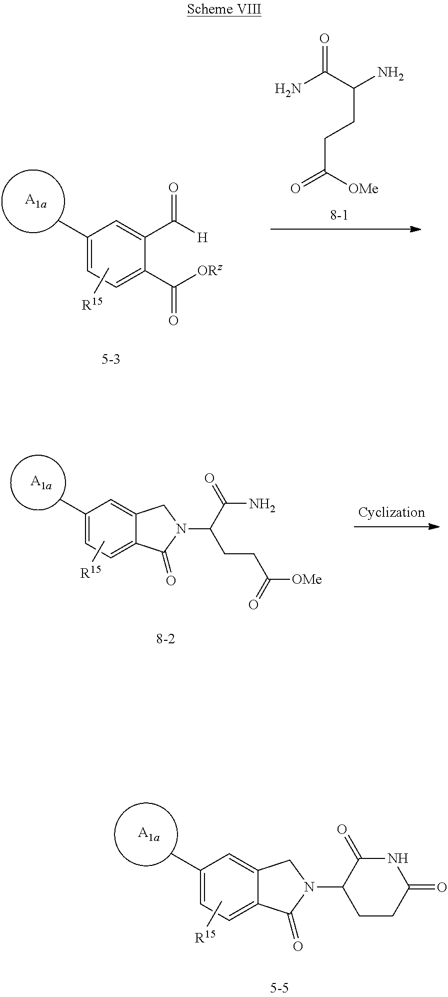

Intermediates for the preparation of compounds of Formula I can be synthesized according to the route described in Scheme IX. Compounds 6-3 can be converted to compounds 9-1 through reductive amination and cyclization with compound 8-1 that is optionally enantioenriched under standard conditions such as in the presence of an appropriate acid (e.g., acetic acid) and a reducing agent (e.g., sodium triacetoxyborohydride) and then a base (e.g., N,N-diisopropylethylamine). Cyclization of 9-1 in the presence of a base (e.g., potassium tert-butoxide) can afford compounds 6-4.

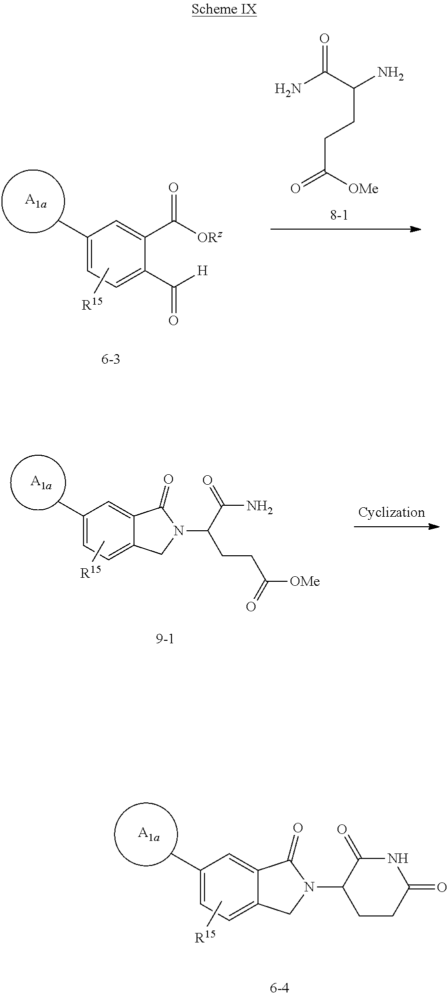

### Example 1. N-(4-((1H-Pyrazol-1-yl)methyl)-2,3-dihydrobenzofuro[7,6-d]isoxazol-8-yl)-3-(7-(((S)-1-(2-((S)-2,6-dioxopiperidin-3-yl)-1-oxoisoindolin-5-yl)pyrrolidin-3-yl)methyl)-2,7-diazaspiro[3.5]nonan-2-yl)benzenesulfonamide

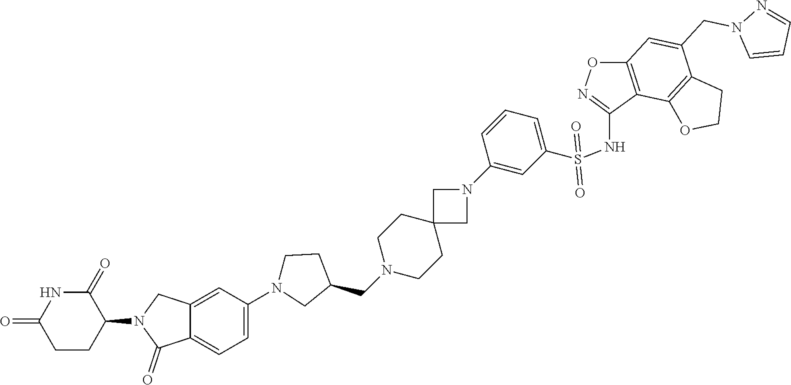 [SMILES: O=C1CC[C@H](N2Cc3cc(N4CC[C@@H](CN5CCC6(CC5)CN(c5cccc(S(=O)(=O)Nc7noc8cc(Cn9cccn9)c9c(c78)OCC9)c5)C6)C4)ccc3C2=O)C(=O)N1]

### Step 1. Methyl (R)-2-cyano-4-(3-(hydroxymethyl)pyrrolidin-1-yl)benzoate

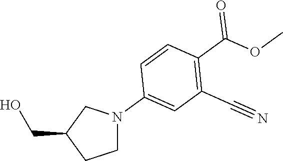 [SMILES: COC(=O)c1ccc(N2CC[C@@H](CO)C2)cc1C#N]

To a solution of methyl 2-cyano-4-fluorobenzoate (600 mg, 3.35 mmol) and (R)-pyrrolidin-3-ylmethanol (508 mg, 5.02 mmol) in DMSO (8 mL) was added N,N-diisopropyl-ethylamine (2.33 mL, 13.4 mmol). The reaction mixture was stirred at 100° C. for 2 h. The reaction mixture was cooled to room temperature and diluted with water (15 mL) and EtOAc (15 mL). The layers were separated, and the aqueous layer was extracted with EtOAc (3×15 mL). The combined organic layers were washed with brine, dried over MgSO₄, filtered, and concentrated to afford the title compound (820 mg, 3.2 mmol, 94% yield) as a tan oil. LC-MS calc. for C₁₄H₁₇N₂O₃ [M+H]⁺: m/z=261.1; Found: 261.0.

### Step 2. Methyl (R)-2-cyano-4-(3-formylpyrrolidin-1-yl)benzoate

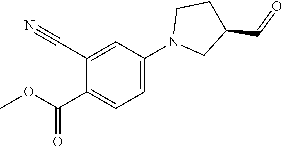 [SMILES: COC(=O)c1ccc(N2CC[C@@H](C=O)C2)cc1C#N]

To a solution of methyl 2-cyano-4-[(3R)-3-(hydroxymethyl)pyrrolidin-1-yl]benzoate (15.7 g, 60.1 mmol) in DCM (160 mL) was added Dess-Martin periodinane (28.1 g, 66.1 mmol). The reaction mixture was stirred at 30° C. for 2 h. The mixture was quenched with 10% sodium thiosulfate/8% sodium bicarbonate (aq.) (160 mL) and stirred for 1 h. The mixture was diluted with water (100 mL) and extracted with DCM (150 mL). The aqueous layer was filtered and extracted with DCM (120 mL×2). The combined organic layers were washed with sat. NaHCO₃ (aq.) (200 mL×2) and brine (200 mL), dried over Na₂SO₄, filtered, and concentrated to afford the title compound (1.10 g, 4.26 mmol, >99% yield) as an orange oil. LC-MS calc. for C₁₄H₁₅N₂O₃ [M+H]⁺: m/z=259.1; Found: 259.5.

### Step 3. Methyl (R)-2-cyano-4-(3-(dimethoxymethyl)pyrrolidin-1-yl)benzoate

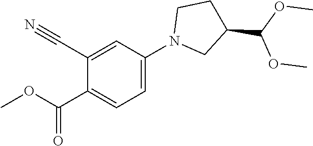

To a solution of methyl (R)-2-cyano-4-(3-(dimethoxymethyl)pyrrolidin-1-yl)benzoate (17.4 g, 67.4 mmol) in methanol (100 mL) was added trimethyl orthoformate (36.9 mL, 337 mmol) and p-toluenesulfonic acid (1.28 g, 6.74 mmol). The resulting mixture was stirred at 60° C. for 1 h. 10% Na₂CO₃ (aq.) (170 mL) was added, and the mixture was stirred for 15 min. The reaction mixture was diluted with water (150 mL) and extracted with EtOAc (200 mL×3). The combined organic layers were washed with brine, dried over Na₂SO₄, filtered, and concentrated. The crude material was purified by silica gel chromatography (0-20% EtOAc/hexanes) to afford the title compound (14.1 g, 46.3 mmol, 68.8% yield) as an orange oil. LC-MS calc. for C₁₆H₂₁N₂O₄ [M+H]⁺: m/z=305.1; Found: 305.6.

### Step 4. Methyl (R)-4-(3-(dimethoxymethyl)pyrrolidin-1-yl)-2-formylbenzoate

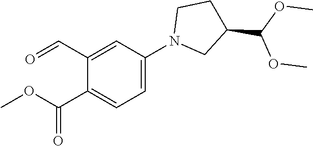

To a solution of methyl (R)-2-cyano-4-(3-(dimethoxymethyl)pyrrolidin-1-yl)benzoate (5.6 g, 18 mmol) in acetic acid (60 mL) was added pyridine (2.18 g, 27.6 mmol). Sodium hypophosphate monohydrate (9.75 g, 92.0 mmol), NaOH (1.47 g, 36.9 mmol), and Raney nickel (2.39 g, 36.8 mmol) were added. The reaction mixture was stirred at 60° C. for 2 h. The mixture was diluted with DCM (200 mL) and filtered. The filtrate was diluted with water (200 mL) and extracted with DCM (200 mL×3). The combined organic layers were washed with brine (300 mL×3), dried over Na₂SO₄, filtered, and concentrated. The crude material was purified by silica gel chromatography (0-17% EtOAc/hexanes) to afford the title compound (1.10 g, 3.58 mmol, 19.5% yield) as a yellow solid. LC-MS calc. for C₁₆H₂₂NO₅ [M+H]⁺: m/z=308.1; Found: 308.6.

### Step 5. Methyl (S)-5-amino-4-(5-((R)-3-(dimethoxymethyl)pyrrolidin-1-yl)-1-oxoisoindolin-2-yl)-5-oxopentanoate

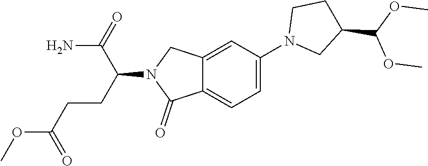

To a mixture of methyl (R)-4-(3-(dimethoxymethyl)pyrrolidin-1-yl)-2-formylbenzoate (0.77 g, 3.9 mmol) in DMF (2 mL) was added N,N-diisopropylethylamine (1.26 g, 9.76 mmol). The reaction mixture was stirred for 10 min. A solution of methyl 4-[(3R)-3-(dimethoxy-methyl)pyrrolidin-1-yl]-2-formylbenzoate (1.00 g, 3.25 mmol) in DCM (10 mL) and then acetic acid (0.98 g, 16 mmol) were added. The reaction mixture was stirred at 40° C. for 1 h. Sodium triacetoxyborohydride (1.38 g, 6.51 mmol) was added, and the reaction mixture was stirred at room temperature for 2 h. The reaction mixture was poured into sat. NaHCO₃ (aq.) (30 mL) and extracted with DCM (30 mLyes×3). The combined organic layers were washed with brine (25 mL×3), dried over Na₂SO₄, filtered, and concentrated. Purification by silica gel chromatography (0-75% EtOAc/hexanes) afforded the title compound (900 mg, 2.15 mmol, 65.9% yield) as a light-yellow solid. LC-MS calc. for C₂₁H₃₀N₃O₆[M+H]⁺: m/z=420.2; Found: 420.6.

### Step 6. (S)-3-(5-((R)-3-(Dimethoxymethyl)pyrrolidin-1-yl)-1-oxoisoindolin-2-yl) piperidine-2, 6-dione

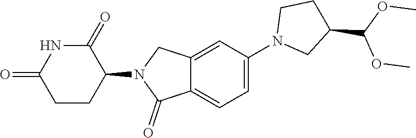 [SMILES: COC(OC)[C@@H]1CCN(c2ccc3c(c2)CN([C@H]2CCC(=O)NC2=O)C3=O)C1]

To a solution of methyl (S)-5-amino-4-(5-((R)-3-(dimethoxymethyl)pyrrolidin-1-yl)-1-oxoisoindolin-2-yl)-5-oxopentanoate (900 mg, 2.15 mmol) in THF (18 mL) at −78° C. was added a solution of potassium tert-butoxide (253 mg, 2.25 mmol) in THF (5 mL) dropwise. The resulting mixture was stirred at 0° C. for 1 h. The reaction mixture was quenched with 1 N HCl (9 mL) and stirred at room temperature for 10 min. The mixture was adjusted to pH˜6 with sat. NaHCO₃ (aq.). The mixture was diluted with water (40 mL) and extracted with EtOAc (50 mL×3). The combined organic layers were dried over Na₂SO₄, filtered, and concentrated to afford the title compound (770 mg, 1.99 mmol, 92.6% yield) as a light-yellow solid. LC-MS calc. for C₂₀H₂₆N₃O₅ [M+H]⁺: m/z=388.2; Found: 388.7.

### Step 7. (R)-1-(2-((S)-2,6-Dioxopiperidin-3-yl)-1-oxoisoindolin-5-yl)pyrrolidine-3-carbaldehyde

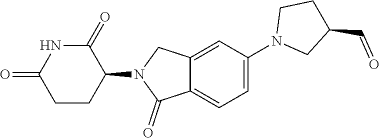 [SMILES: O=C[C@@H]1CCN(c2ccc3c(c2)CN([C@H]2CCC(=O)NC2=O)C3=O)C1]

To a solution of (S)-3-(5-((R)-3-(dimethoxymethyl)pyrrolidin-1-yl)-1-oxoisoindolin-2-yl)piperidine-2,6-dione (100 mg, 0.258 mmol) in acetone (1.5 mL) was added HCl (1.55 mL, 3.1 mmol, 2N in 1,4-dioxane). The reaction was stirred for 3 h. The reaction mixture was diluted with water (3 mL) and EtOAc (3 mL). The layers were separated, and the aqueous layer was extracted with EtOAc (3×5 mL). The combined organic layers were washed with brine, dried over MgSO₄, filtered, and concentrated to afford the title compound (58 mg, 0.17 mmol, 66% yield) as a yellow solid. LC-MS calc. for C₁₈H₂₀N₃O₄[M+H]⁺: m/z=342.1; Found: 342.1.

### Step 6. N-(4-((1H-Pyrazol-1-yl)methyl)-2,3-dihydrobenzofuro[7,6-d]isoxazol-8-yl)-3-bromobenzenesulfonamide

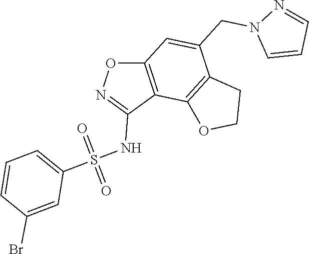 [SMILES: O=S(=O)(Nc1noc2cc(Cn3cccn3)c3c(c12)OCC3)c1cccc(Br)c1]

To a solution of 4-((1H-pyrazol-1-yl)methyl)-2,3-dihydrobenzofuro[7,6-d]isoxazol-8-amine (200 mg, 0.780 mmol) (prepared as described by Venkateshappa, et al. WO2023/114710 A1) and 3-bromobenzenesulfonyl chloride (499 mg, 1.95 mmol) in THF (5.5 mL) was added potassium t-butoxide (701 mg, 6.24 mmol). The reaction mixture was stirred for 1 h. The reaction mixture was adjusted to pH -3 with 1 N HCl and then diluted with water (15 mL) and EtOAc (15 mL). The aqueous layer was extracted with EtOAc (3×10 mL). The combined organic layers were washed with brine, dried over MgSO₄, filtered, and concentrated. The material was purified by silica gel chromatography (50-100% EtOAc/hexanes) to afford the title compound (228 mg, 0.480 mmol, 61.5% yield) as a tan solid. LC-MS calc. for C₁₉H₁₆BrN₄O₄S [M+H]⁺: m/z=475.0, 477.0; Found: 474.9, 477.0.

### Step 7. N-(4-((1H-Pyrazol-1-yl)methyl)-2,3-dihydrobenzofuro[7,6-d]isoxazol-8-yl)-3-(2,7-diazaspiro[3.5]nonan-2-yl)benzenesulfonamide

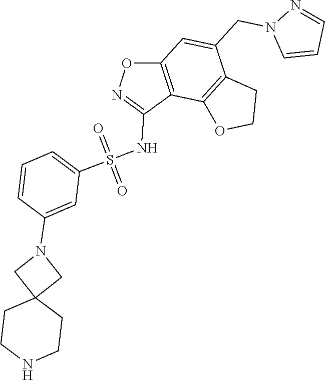 [SMILES: O=S(=O)(Nc1noc2cc(Cn3cccn3)c3c(c12)OCC3)c1cccc(N2CC3(CCNCC3)C2)c1]

To a nitrogen sparged mixture of N-(4-((1H-pyrazol-1-yl)methyl)-2,3-dihydrobenzo-furo[7,6-d]isoxazol-8-yl)-3-bromobenzenesulfonamide (41 mg, 0.087 mmol), tert-butyl 2,7-diazaspiro[3.5]nonane-7-carboxylate hydrochloride (27 mg, 0.10 mmol), and sodium tert-butoxide (42 mg, 0.43 mmol) was added BrettPhos Pd G₃ (6.3 mg, 0.0070 mmol, CAS 1470372-59-8). The reaction mixture was stirred for 1 h at 100° C. under a nitrogen atmosphere. The reaction mixture was concentrated, and TFA (0.75 mL) was added. The reaction mixture was stirred for 30 min and then concentrated. The crude material was purified by silica gel chromatography (0-20% MeOH/DCM) to afford the title compound (81 mg, 0.16 mmol, 99% yield) as a colorless solid. LC-MS calc. for C₂₆H₂₉N₆O₄S [M+H]⁺: m/z=521.2; Found: 521.1.

### Step 8. N-(4-((1H-Pyrazol-1-yl)methyl)-2,3-dihydrobenzofuro[7,6-d]isoxazol-8-yl)-3-(7-(((S)-1-(2-((S)-2,6-dioxopiperidin-3-yl)-1-oxoisoindolin-5-yl)pyrrolidin-3-yl)methyl)-2,7-diazaspiro[3.5]nonan-2-yl)benzenesulfonamide

To a mixture of (R)-1-(2-((S)-2,6-dioxopiperidin-3-yl)-1-oxoisoindolin-5-yl)pyrrolidine-3-carbaldehyde (14 mg, 0.042 mmol) and N-(4-((1H-pyrazol-1-yl)methyl)-2,3-dihydrobenzofuro[7,6-d]isoxazol-8-yl)-3-(2,7-diazaspiro[3.5]nonan-2-yl)benzenesulfonamide (22 mg, 0.042 mmol) in DMSO (1.2 mL) and MeCN (0.4 mL) was added acetic acid (36 μL, 0.63 mmol). The reaction was stirred for 0.5 h. Sodium triacetoxyborohydride (36 mg, 0.17 mmol) was added, and the reaction mixture was stirred for 1 h. The reaction mixture was diluted with MeCN, filtered, and purified by prep-HPLC on a C18 column (23-43% MeCN/0.1% TFA (aq)) to the title compound as a TFA salt (11 mg, 0.010 mmol, 25% yield), a white solid. ¹H NMR (400 MHz, DMSO-d₆) δ 10.94 (s, 1H), 7.84 (d, J=2.3 Hz, 1H), 7.57-7.47 (m, 2H), 7.39 (t, J=7.9 Hz, 1H), 7.23 (d, J=7.7 Hz, 1H), 6.95 (s, 1H), 6.78-6.62 (m, 4H), 6.31 (d, J=2.1 Hz, 1H), 5.43 (s, 2H), 5.05 (dd, J=13.2, 5.1 Hz, 1H), 4.79 (t, J=8.9 Hz, 2H), 4.40-4.14 (m, 2H), 3.62-3.43 (m, 8H), 3.40-3.22 (m, 3H), 3.13 (t, J=9.0 Hz, 2H), 2.52-2.48 (m, 12H), 2.43-2.22 (m, 1H), 2.15 (d, J=13.5 Hz, 1H), 2.05-1.91 (m, 3H), 1.83 (d, J=10.5 Hz, 1H). LC-MS calc. for C₄₄H₄₈N₉O₇S [M+H]⁺: m/z=846.3; Found: 846.2.

### Examples 2-5

Examples 2-5 listed in Tables 1 and 2 were synthesized according to procedures analogous to Example 1. All examples in Tables 1 and 2 were prepared as TFA salts. In Table 1 below, * represents connection of Y¹ to the 3-methylpyrrolidinyl moiety and [symbol] represents connection of Y¹ to ring A of the PTM.

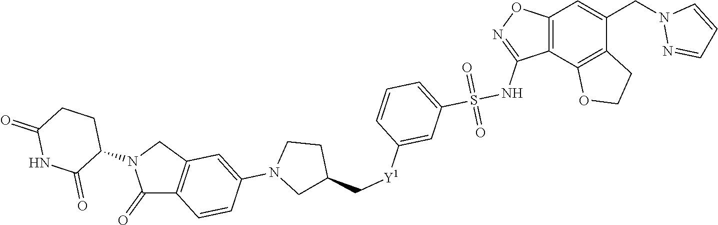

TABLE 1
Examples 2-5

| | | LCMS |
|---|---|---|
| Ex. | Y¹ | [M + H]⁺ |
| 2 | 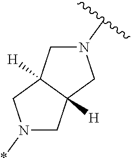 [SMILES: *N1C[C@H]2CN(*)C[C@@H]2C1] | 832.4 |
| 3 | 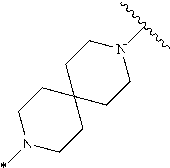 [SMILES: *N1CCC2(CC1)CCN(*)CC2] | 874.3 |
| 4 | 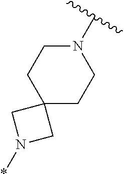 [SMILES: *N1CCC2(CC1)CN(*)C2] | 846.4 |
| 5 | 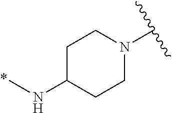 [SMILES: *N1CCC(NC)CC1] | 820.3 |

TABLE 2
Examples 2-5

| Ex. | Compound Name | NMR |
|---|---|---|
| 2 | N-(4-((1H-Pyrazol-1-yl)methyl)-2,3- | ¹H NMR (400 MHz, DMSO-d₆) δ |
| | dihydrobenzofuro[7,6-d]isoxazol-8-yl)-3- | 11.22 (s, 1H), 10.93 (s, 1H), 10.52 (s, |
| | ((3aS,6aS)-5-(((S)-1-(2-((S)-2,6- | 1H), 7.83 (d, J = 2.3 Hz, 1H), 7.57- |
| | dioxopiperidin-3-yl)-1-oxoisoindolin-5- | 7.45 (m, 2H), 7.37 (t, J = 8.0 Hz, 1H), |
| | yl)pyrrolidin-3-yl)methyl)hexahydro- | 7.16 (d, J = 7.7 Hz, 1H), 7.05 (s, 1H), |
| | pyrrolo[3,4-c]pyrrol-2(1H)- | 6.77 (d, J = 8.5 Hz, 1H), 6.69 (s, 1H), |
| | yl)benzenesulfonamide | 6.66-6.59 (m, 2H), 6.30 (t, J = 2.1 Hz, |
| | | 1H), 5.42 (s, 2H), 5.04 (dd, J = 13.4, |
| | | 5.1 Hz, 1H), 4.78 (t, J = 8.9 Hz, 2H), |
| | | 4.37-4.12 (m, 2H), 3.95-3.86 (m, 1H), |
| | | 3.64-3.53 (m, 2H), 3.38-3.28 (m, 1H), |
| | | 3.22-3.05 (m, 6H), 2.95-2.57 (m, 4H), |
| | | 2.51-2.45 (m, 6H), 2.40-2.29 (m, 2H), |
| | | 2.29-2.19 (m, 1H), 2.01-1.90 (m, 1H), |
| | | 1.87-1.76 (m, 1H) |
| 3 | N-(4-((1H-Pyrazol-1-yl)methyl)-2,3- | — |
| | dihydrobenzofuro[7,6-d]isoxazol-8-yl)-3- | |
| | (9-(((S)-1-(2-((S)-2,6-dioxopiperidin-3-yl)- | |
| | 1-oxoisoindolin-5-yl)pyrrolidin-3- | |
| | yl)methyl)-3,9-diazaspiro[5.5]undecan-3- | |
| | yl)benzenesulfonamide | |
| 4 | N-(4-((1H-Pyrazol-1-yl)methyl)-2,3- | — |
| | dihydrobenzofuro[7,6-d]isoxazol-8-yl)-3- | |
| | (2-(((S)-1-(2-((S)-2,6-dioxopiperidin-3-yl)- | |
| | 1-oxoisoindolin-5-yl)pyrrolidin-3- | |
| | yl)methyl)-2,7-diazaspiro[3.5]nonan-7- | |
| | yl)benzenesulfonamide | |
| 5 | N-(4-((1H-Pyrazol-1-yl)methyl)-2,3- | — |
| | dihydrobenzofuro[7,6-d]isoxazol-8-yl)-3- | |
| | (4-((((S)-1-(2-((S)-2,6-dioxopiperidin-3- | |
| | yl)-1-oxoisoindolin-5-yl)pyrrolidin-3- | |
| | yl)methyl)amino)piperidin-1- | |
| | yl)benzenesulfonamide | |

### Example 6. N-(4-((1H-Pyrazol-1-yl)methyl)-2,3-dihydrobenzofuro[7,6-d]isoxazol-8-yl)-3-(4-(7-(2-(2,6-dioxopiperidin-3-yl)-1,3-dioxoisoindolin-5-yl)-7-azaspiro[3.5]nonan-2-yl)piperazin-1-yl)benzenesulfonamide

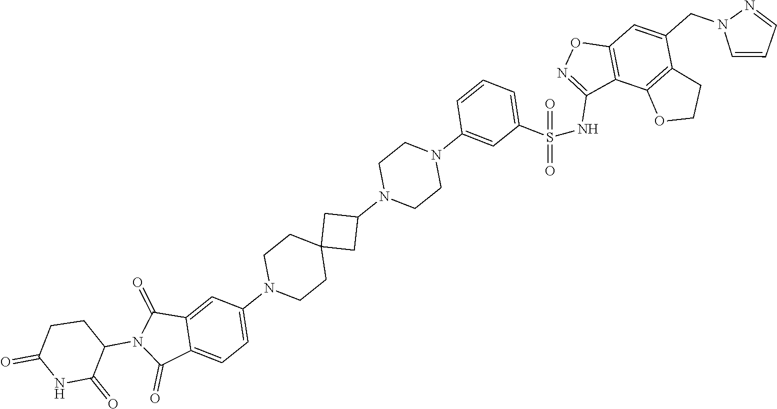 [SMILES: O=C1CCC(N2C(=O)c3ccc(N4CCC5(CC4)CC(N4CCN(c6cccc(S(=O)(=O)Nc7noc8cc(Cn9cccn9)c9c(c78)OCC9)c6)CC4)C5)cc3C2=O)C(=O)N1]

### Step 1. 2-(2,6-Dioxopiperidin-3-yl)-5-(2-hydroxy-7-azaspiro[3.5]nonan-7-yl)iso-indoline-1,3-dione

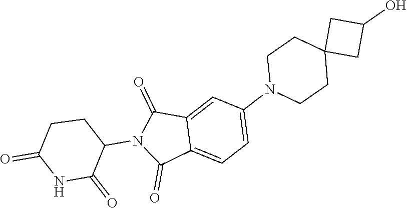 [SMILES: O=C1CCC(N2C(=O)c3ccc(N4CCC5(CC4)CC(O)C5)cc3C2=O)C(=O)N1]

To a solution of 2-(2,6-dioxopiperidin-3-yl)-5-fluoroisoindoline-1,3-dione (500 mg, 1.81 mmol) (Combi-Blocks, HD-3240) and 7-azaspiro[3.5]nonan-2-ol hydrochloride (322 mg, 1.81 mmol) in NMP (7.5 mL) was added N,N-diisopropylethylamine (0.95 mL, 5.4 mmol). The reaction was stirred for 48 h at 100° C. The reaction was cooled to room temperature and diluted with water (30 mL) and EtOAc (30 mL). The layers were separated, and the aqueous was extracted with EtOAc (3×25 mL). The combined organic layers were washed with brine, dried over MgSO₄, filtered, and concentrated. The crude material was purified by silica gel chromatography (50-100% EtOAc/hexanes) to afford the title compound (1.03 g, 2.59 mmol, 99% yield) as a yellow oil. LC-MS calc. for C₂₁H₂₄N₃O₅ [M+H]⁺: m/z=398.2; Found: 398.4.

### Step 2. N-(4-((1H-Pyrazol-1-yl)methyl)-2,3-dihydrobenzofuro[7,6-d]isoxazol-8-yl)-3-(piperazin-1-yl)benzenesulfonamide

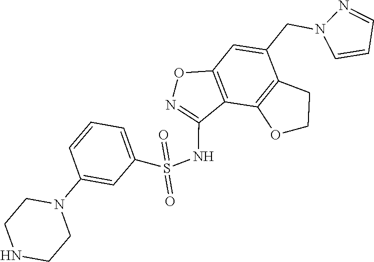 [SMILES: O=S(=O)(Nc1noc2cc(Cn3cccn3)c3c(c12)OCC3)c1cccc(N2CCNCC2)c1]

The title compound was synthesized according to procedures analogous to Example 1, Step 7. LC-MS calc. for C₂₃H₂₅N₆O₄S [M+H]⁺: m/z=481.2; Found 481.1.

### Step 3. N-(4-((1H-Pyrazol-1-yl)methyl)-2,3-dihydrobenzofuro[7,6-d]isoxazol-8-yl)-3-(4-(7-(2-(2,6-dioxopiperidin-3-yl)-1,3-dioxoisoindolin-5-yl)-7-azaspiro[3.5]nonan-2-yl)piperazin-1-yl)benzenesulfonamide

To a solution of 2-(2,6-dioxopiperidin-3-yl)-5-(2-hydroxy-7-azaspiro[3.5]nonan-7-yl)isoindole-1,3-dione (17 mg, 0.042 mmol) and triethylamine (0.052 mL, 0.37 mmol) in DMSO (0.5 mL) was added a solution of sulfur trioxide pyridine complex (20 mg, 0.12 mmol) in DMSO (0.5 mL). The reaction was stirred for 1 h. A solution of N-(4-((1H-pyrazol-1-yl) methyl)-2,3-dihydrobenzofuro[7,6-d]isoxazol-8-yl)-3-(piperazin-1-yl)benzenesulfonamide (20 mg, 0.042 mmol) in MeCN (0.3 mL) and DMSO (0.3 mL) and then acetic acid (0.036 mL, 0.62 mmol) were added. The reaction mixture was stirred for 0.5 h. Sodium triacetoxyboro-hydride (35 mg, 0.17 mmol) was added, and the reaction mixture was stirred for 1 h. The reaction was quenched with water (0.5 mL). The reaction mixture was diluted with MeCN, filtered, and purified by prep-HPLC on a C₁₈ column (28-48% MeCN/0.1% TFA (aq)) to afford the title compound as a TFA salt (1.5 mg, 0.0015 mmol, 3.5% yield), a yellow solid. LC-MS calc. for C₄₄H₄₆N₉O₈S [M+H]⁺: m/z=860.3; Found: 860.4.

### Example 7. N-(4-((1H-Pyrazol-1-yl)methyl)-2,3-dihydrobenzofuro[7,6-d]isoxazol-8-yl)-3-(4-((1-(2-(2,6-dioxopiperidin-3-yl)-1,3-dioxoisoindolin-4-yl)piperidin-4-yl)methyl) piperazin-1-yl)benzenesulfonamide

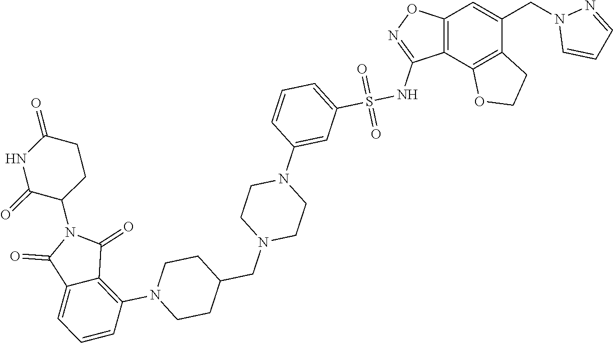 [SMILES: O=C1CCC(N2C(=O)c3cccc(N4CCC(CN5CCN(c6cccc(S(=O)(=O)Nc7noc8cc(Cn9cccn9)c9c(c78)OCC9)c6)CC5)CC4)c3C2=O)C(=O)N1]

### Step 1. 2-(2,6-Dioxopiperidin-3-yl)-4-(4-(hydroxymethyl)piperidin-1-yl)isoindoline-1,3-dione

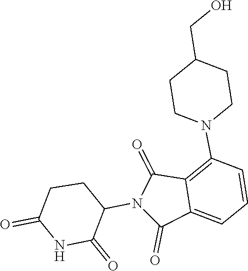 [SMILES: O=C1CCC(N2C(=O)c3cccc(N4CCC(CO)CC4)c3C2=O)C(=O)N1]

To a solution of 2-(2,6-dioxo-piperidin-3-yl)-4-fluoroisoindoline-1,3-dione (500 mg, 1.81 mmol) (Combi-Blocks, HB-2696) and 4-piperidinemethanol (271 mg, 2.35 mmol) in NMP (7.5 mL) was added N,N-diisopropylethylamine (0.95 mL, 5.4 mmol). The reaction mixture was stirred for 48 h at 100° C. The reaction mixture was cooled to room temperature and diluted with water (30 mL) and EtOAc (30 mL). The layers were separated, and the aqueous was extracted with EtOAc (3×25 mL). The combined organic layers were washed with brine, dried over MgSO₄, filtered, and concentrated. The crude material was purified by silica gel chromatography (50-100% EtOAc/hexanes) to afford the title compound (842 mg, 2.27 mmol, 125% yield) as a yellow oil. LC-MS calc. for C₁₉H₂₂N₃O₅ [M+H]⁺: m/z=372.2; Found: 372.1.

### Step 2. N-(4-((1H-Pyrazol-1-yl)methyl)-2,3-dihydrobenzofuro[7,6-d]isoxazol-8-yl)-3-(4-((1-(2-(2,6-dioxopiperidin-3-yl)-1,3-dioxoisoindolin-4-yl)piperidin-4-yl)methyl)piperazin-1-yl)benzenesulfonamide

The title compound was synthesized according to procedures analogous to Example 6, Step 3. ¹H NMR (400 MHz, DMSO-d₆) δ 11.08 (s, 1H), 7.84 (d, J=2.3 Hz, 1H), 7.71 (t, J=7.7 Hz, 1H), 7.60-7.25 (m, 7H), 6.70 (s, 1H), 6.31 (d, J=2.1 Hz, 1H), 5.43 (s, 2H), 5.09 (dd, J=12.6, 5.5 Hz, 1H), 4.80 (t, J=8.9 Hz, 2H), 3.94-3.60 (m, 5H), 3.19-3.05 (m, 9H), 2.94 (t, J=12.8 Hz, 4H), 2.71-2.61 (m, 1H), 2.16-2.00 (m, 2H), 1.90 (d, J=12.6 Hz, 2H), 1.62-1.38 (m, 2H). LC-MS calc. for C₄₂H₄₄N₉O₈S [M+H]⁺: m/z=834.3; Found 834.3.

### Example 8. N-(4-((1H-Pyrazol-1-yl)methyl)-2,3-dihydrobenzofuro[7,6-d]isoxazol-8-yl)-3-(4-((1-((1-(2-(2,6-dioxopiperidin-3-yl)-1,3-dioxoisoindolin-5-yl)pyrrolidin-3-yl)methyl) piperidin-4-yl)methyl)piperazin-1-yl)benzenesulfonamide

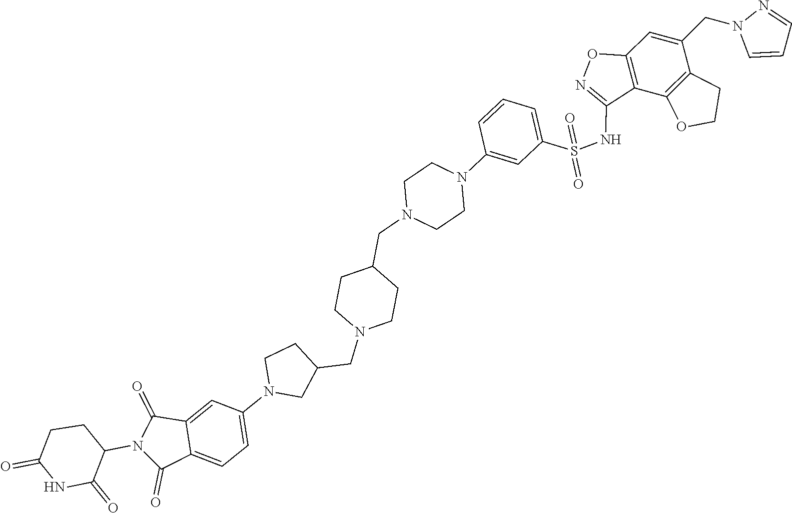 [SMILES: O=C1CCC(N2C(=O)c3ccc(N4CCC(CN5CCC(CN6CCN(c7cccc(S(=O)(=O)Nc8noc9cc(Cn%10cccn%10)c%10c(c89)OCC%10)c7)CC6)CC5)C4)cc3C2=O)C(=O)N1]

### Step 1. 2-(2,6-Dioxopiperidin-3-yl)-5-(3-(hydroxymethyl)pyrrolidin-1-yl)isoindoline-1,3-dione

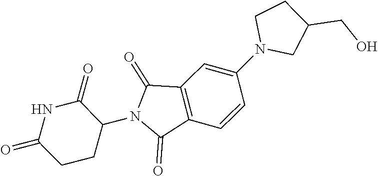 [SMILES: O=C1CCC(N2C(=O)c3ccc(N4CCC(CO)C4)cc3C2=O)C(=O)N1]

To a solution of 2-(2,6-dioxo-3-piperidinyl)-5-fluoro-1H-isoindole-1,3(2H)-dione (450 mg, 1.63 mmol) and pyrrolidin-3-ylmethanol (214 mg, 2.12 mmol) in NMP (8 mL) was added N,N-diisopropylethylamine (0.850 mL, 4.89 mmol). The reaction was stirred for 48 h at 100° C. The reaction mixture was cooled to room temperature and diluted with water (30 mL) and EtOAc (30 mL). The layers were separated, and the aqueous layer was extracted with EtOAc (3×25 mL). The combined organic layers were washed with brine, dried over MgSO₄, filtered, and concentrated. The crude material was purified by silica gel chromatography (50-100% EtOAc/hexanes) to the title compound (0.581 g, 1.63 mmol, 99.7% yield) as a yellow oil. LC-MS calc. for C₁₈H₂₀N₃O₅ [M+H]⁺: m/z=358.1; Found: 358.0.

### Step 2. 2-(2,6-Dioxopiperidin-3-yl)-5-(3-((4-(hydroxymethyl)piperidin-1-yl)methyl) pyrrolidin-1-yl)isoindoline-1,3-dione

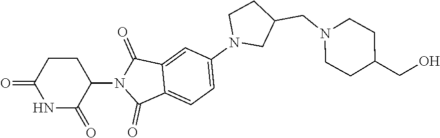 [SMILES: O=C1CCC(N2C(=O)c3ccc(N4CCC(CN5CCC(CO)CC5)C4)cc3C2=O)C(=O)N1]

To a solution of 2-(2,6-dioxopiperidin-3-yl)-5-[3-(hydroxymethyl)pyrrolidin-1-yl]isoindole-1,3-dione (144 mg, 0.404 mmol) in DMSO (4 mL) was added triethylamine (0.507 mL, 3.63 mmol). A solution of sulfur trioxide pyridine complex (193 mg, 1.21 mmol) in DMSO (4 mL) was added. The reaction mixture was stirred for 1 h. A solution of 4-piperidinemethanol (70 mg, 0.61 mmol) in DMSO (4 mL) and then acetic acid (0.350 mL, 6.06 mmol) were added. After stirring for 0.5 h, sodium triacetoxyborohydride (342 mg, 1.62 mmol) was added. The reaction mixture was stirred for 1 h. The reaction was quenched with water (0.5 mL). The reaction mixture was diluted with MeCN, filtered, and purified by prep-HPLC on a C₁₈ column (10-30% MeCN/0.1% TFA (aq)) to afford the title compound (30 mg, 0.066 mmol, 16% yield) as a yellow solid. LC-MS calc. for C₂₄H₃₁N₄O₅ [M+H]⁺: m/z=455.2; Found: 455.0.

### Step 3. N-(4-((1H-Pyrazol-1-yl)methyl)-2,3-dihydrobenzofuro[7,6-d]isoxazol-8-yl)-3-(4-((1-((1-(2-(2,6-dioxopiperidin-3-yl)-1,3-dioxoisoindolin-5-yl)pyrrolidin-3-yl)methyl) piperidin-4-yl)methyl)piperazin-1-yl)benzenesulfonamide

The title compound was synthesized according to procedures analogous to Example 6, Step 3. ¹H NMR (400 MHz, DMSO-d₆) δ 11.07 (s, 1H), 7.84 (s, 1H), 7.70 (d, J=8.6 Hz, 1H), 7.58-7.45 (m, 3H), 7.43 (s, 1H), 7.32 (s, 1H), 6.95 (s, 1H), 6.85 (d, J=8.0 Hz, 1H), 6.70 (s, 1H), 6.31 (s, 1H), 5.43 (s, 1H), 5.13-4.98 (m, 2H), 4.80 (t, J=8.9 Hz, 1H), 3.78-3.55 (m, 2H), 3.20-3.03 (m, 12H), 3.03-2.76 (m, 6H), 2.60 (d, J=14.7 Hz, 3H), 2.30 (d, J=25.6 Hz, 2H), 2.11-1.54 (m, 8H), 1.49 (s, 3H). LC-MS calc. for C₄₇H₅₃N₁₀O₈S [M+H]⁺: m/z=917.4; Found 917.7.

### Example 9. N-(4-((1H-Pyrazol-1-yl)methyl)-2,3-dihydrobenzofuro[7,6-d]isoxazol-8-yl)-3-(4-((1-((1-(2-(2,6-dioxopiperidin-3-yl)-1,3-dioxoisoindolin-5-yl)piperidin-4-yl)methyl) piperidin-4-yl)methyl)piperazin-1-yl)benzenesulfonamide

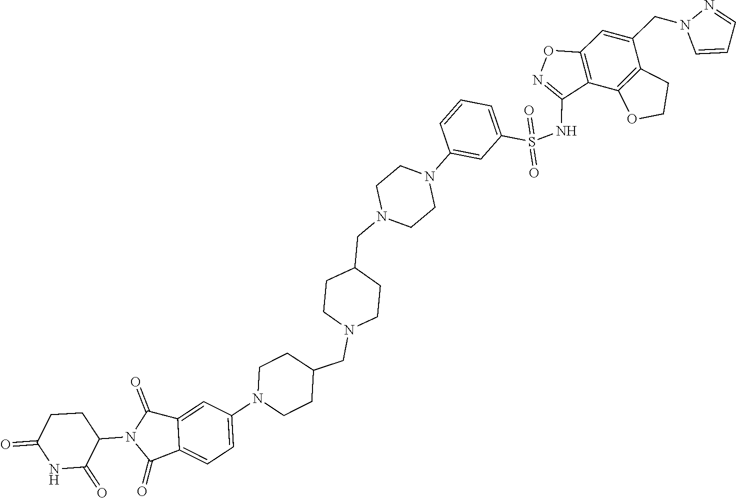 [SMILES: O=C1CCC(N2C(=O)c3ccc(N4CCC(CN5CCC(CN6CCN(c7cccc(S(=O)(=O)Nc8noc9cc(Cn%10cccn%10)c%10c(c89)OCC%10)c7)CC6)CC5)CC4)cc3C2=O)C(=O)N1]

### Step 1. 2-(2,6-Dioxopiperidin-3-yl)-5-(4-((4-(hydroxymethyl)piperidin-1-yl)methyl) piperidin-1-yl)isoindoline-1,3-dione

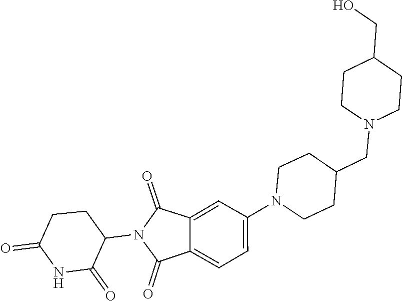 [SMILES: O=C1CCC(N2C(=O)c3ccc(N4CCC(CN5CCC(CO)CC5)CC4)cc3C2=O)C(=O)N1]

The title compound was synthesized according to procedures analogous to Example 8, Steps 1-2. LC-MS calc. for C₂₅H₃₃N₄O₅ [M+H]⁺: m/z=469.2; Found 469.2.

### Step 2. N-(4-((1H-Pyrazol-1-yl)methyl)-2,3-dihydrobenzofuro[7,6-d]isoxazol-8-yl)-3-(4-((1-((1-(2-(2,6-dioxopiperidin-3-yl)-1,3-dioxoisoindolin-5-yl)piperidin-4-yl)methyl) piperidin-4-yl)methyl)piperazin-1-yl)benzenesulfonamide

The title compound was synthesized according to procedures analogous to Example 1, Step 7. LC-MS calc. for C₄₈H₅₅N₁₀O₈S [M+H]⁺: m/z=931.4; Found 931.2.

### Example 10. N-(4-((1H-Pyrazol-1-yl)methyl)-2,3-dihydrobenzofuro[7,6-d]isoxazol-8-yl)-3-(7-(((S)-1-(2-((S)-2,6-dioxopiperidin-3-yl)-1-oxoisoindolin-5-yl)pyrrolidin-3-yl)methyl)-2,7-diazaspiro[3.5]nonan-2-yl)-2,6-dimethoxybenzenesulfonamide

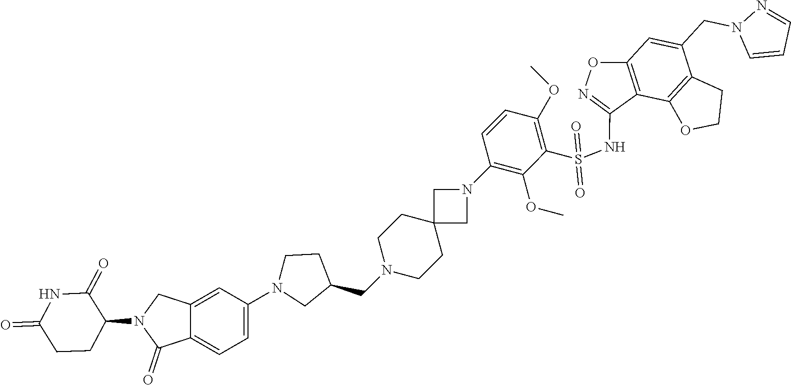 [SMILES: COc1ccc(N2CC3(CCN(C[C@@H]4CCN(c5ccc6c(c5)CN([C@H]5CCC(=O)NC5=O)C6=O)C4)CC3)C2)c(OC)c1S(=O)(=O)Nc1noc2cc(Cn3cccn3)c3c(c12)OCC3]

### Step 1. N-(4-((1H-Pyrazol-1-yl)methyl)-2,3-dihydrobenzofuro[7,6-d]isoxazol-8-yl)-3-bromo-2,6-dimethoxybenzenesulfonamide

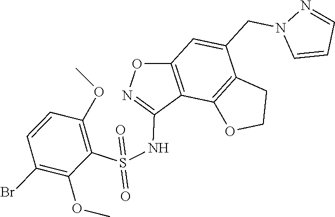 [SMILES: COc1ccc(Br)c(OC)c1S(=O)(=O)Nc1noc2cc(Cn3cccn3)c3c(c12)OCC3]

The title compound was synthesized according to procedures analogous to Example 1, Step 6, using 3-bromo-2,6-dimethoxybenzenesulfonyl chloride. LC-MS calc. for C₂₁H₂₀BrN₄O₆S [M+H]⁺: m/z=535.0, 537.0; Found 535.3, 537.3.

### Step 2. N-(4-((1H-Pyrazol-1-yl)methyl)-2,3-dihydrobenzofuro[7,6-d]isoxazol-8-yl)-2,6-dimethoxy-3-(2,7-diazaspiro[3.5]nonan-2-yl)benzenesulfonamide

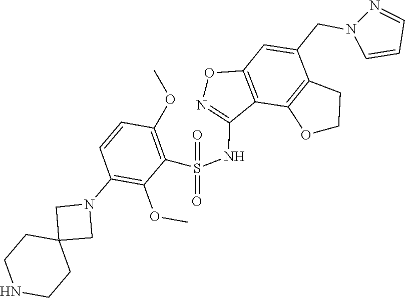 [SMILES: COc1ccc(N2CC3(CCNCC3)C2)c(OC)c1S(=O)(=O)Nc1noc2cc(Cn3cccn3)c3c(c12)OCC3]

The title compound was synthesized according to procedures analogous to Example 1, Step 7. LC-MS calc. for C₂₈H₃₃N₆O₆S [M+H]⁺: m/z=581.2; Found 581.4.

### Step 3. N-(4-((1H-Pyrazol-1-yl)methyl)-2,3-dihydrobenzofuro[7,6-d]isoxazol-8-yl)-3-(7-(((S)-1-(2-((S)-2,6-dioxopiperidin-3-yl)-1-oxoisoindolin-5-yl)pyrrolidin-3-yl)methyl)-2,7-diazaspiro[3.5]nonan-2-yl)-2,6-dimethoxybenzenesulfonamide

The title compound was synthesized according to procedures analogous to Example 1, Step 8. LC-MS calc. for C₄₆H₅₂N₉O₉S [M+H]⁺: m/z=906.4; Found 906.9.

### Example 11. N-(4-((1H-Pyrazol-1-yl)methyl)-2,3-dihydrobenzofuro[7,6-d]isoxazol-8-yl)-3-(7-((1-(2-(2,6-dioxopiperidin-3-yl)-1,3-dioxoisoindolin-5-yl)piperidin-4-yl)methyl)-2,7-diazaspiro[3.5]nonan-2-yl)-2,6-dimethoxybenzenesulfonamide

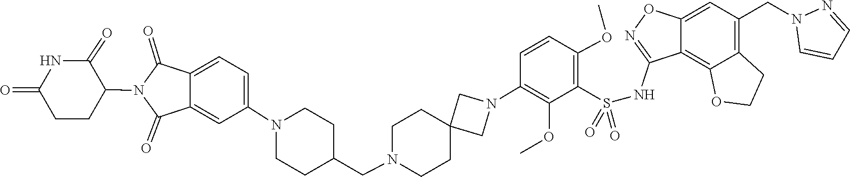 [SMILES: COc1ccc(N2CC3(CCN(CC4CCN(c5ccc6c(c5)C(=O)N(C5CCC(=O)NC5=O)C6=O)CC4)CC3)C2)c(OC)c1S(=O)(=O)Nc1noc2cc(Cn3cccn3)c3c(c12)OCC3]

The title compound was synthesized according to procedures analogous to Example 1, Step 8, using N-(4-((1H-pyrazol-1-yl)methyl)-2,3-dihydrobenzofuro[7,6-d]isoxazol-8-yl)-2,6-dimethoxy-3-(2,7-diazaspiro[3.5]nonan-2-yl)benzenesulfonamide (from Example 10, Step 2). LC-MS calc. for C₄₇H₅₂N₉O₁₀S [M+H]⁺: m/z=934.4; Found 934.9.

### Example 12. N-(4-((1H-Pyrazol-1-yl)methyl)-2,3-dihydrobenzofuro[7,6-d]isoxazol-8-yl)-3-(7-((1-(2-(2,6-dioxopiperidin-3-yl)-1,3-dioxoisoindolin-5-yl)piperidin-4-yl)methyl)-2,7-diazaspiro[3.5]nonan-2-yl)-2-methoxybenzenesulfonamide

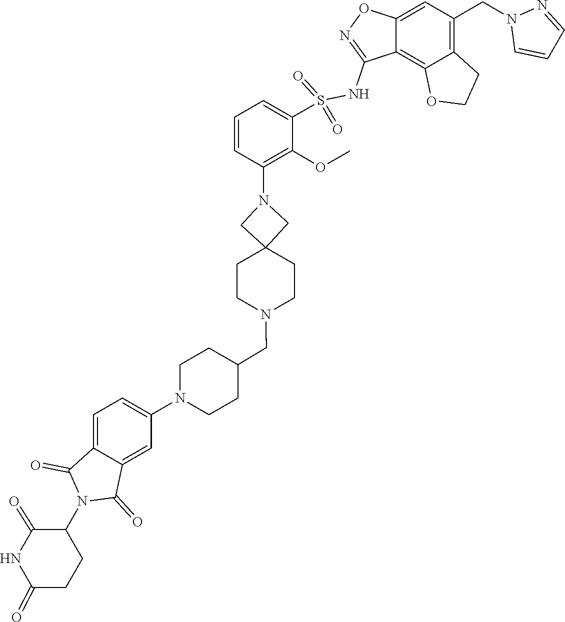 [SMILES: COc1c(N2CC3(CCN(CC4CCN(c5ccc6c(c5)C(=O)N(C5CCC(=O)NC5=O)C6=O)CC4)CC3)C2)cccc1S(=O)(=O)Nc1noc2cc(Cn3cccn3)c3c(c12)OCC3]

### Step 1. Benzyl(3-bromo-2-methoxyphenyl)sulfane

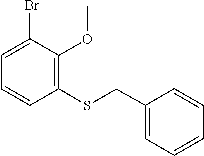 [SMILES: COc1c(Br)cccc1SCc1ccccc1]

To a solution of 1,3-dibromo-2-methoxybenzene (1.76 mg, 6.62 mmol) in 1,4-dioxane (60 mL) was added 9,9-dimethyl-4,5-bis(diphenylphosphino)xanthene (191 mg, 0.331 mmol), tris(dibenzylideneacetone)dipalladium (0) (242 mg, 0.265 mmol), N,N-diisopropylethylamine (2.33 mL, 13.4 mmol), and benzylthiol (0.78 mL, 6.7 mmol). The reaction was stirred at 110° C. for 2 h. The reaction mixture was cooled to room temperature, poured into water (50 mL), and extracted with EtOAc (3×40 mL). The combined organic layers were washed with brine, dried over MgSO₄, filtered, and concentrated. The crude material was purified by silica gel chromatography (0-100% DCM/hexanes) to afford the title compound (1.4 g, 4.6 mmol, 70% yield) as a clear oil. ¹H NMR (400 MHz, CDCl₃) δ 7.50-7.23 (m, 6H), 7.18 (dd, J=7.9, 1.5 Hz, 1H), 6.91 (td, J=7.9, 1.2 Hz, 1H), 4.15 (s, 2H), 3.91 (d, J=1.1 Hz, 3H).

### Step 2. 3-Bromo-2-methoxybenzenesulfonyl chloride

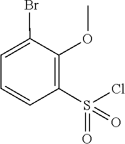

To a solution of benzyl(3-bromo-2-methoxyphenyl)sulfane (1.43 g, 4.62 mmol) in acetic acid (40 mL) was added 1-chloropyrrolidine-2,5-dione (2.47 g, 18.5 mmol). The reaction was stirred for 1 h. The reaction mixture was poured into water and extracted with EtOAc (3×50 mL). The combined organic layers were washed with brine, dried over MgSO₄, filtered, and concentrated. The crude material was purified by silica gel chromatography (0-40% EtOAc/hexanes) to afford the title compound (1.17 g, 4.10 mmol, 88.8% yield) as a clear oil. ¹H NMR (400 MHz, CDCl₃) δ 7.96 (td, J=8.2, 1.6 Hz, 2H), 7.22 (t, J=8.0 Hz, 1H), 4.16 (s, 3H).

### Step 3. N-(4-((1H-Pyrazol-1-yl)methyl)-2,3-dihydrobenzofuro[7,6-d]isoxazol-8-yl)-2-methoxy-3-(2,7-diazaspiro[3.5]nonan-2-yl)benzenesulfonamide

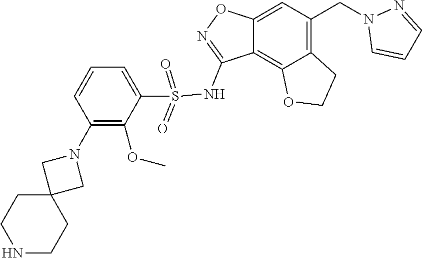 [SMILES: COc1c(N2CC3(CCNCC3)C2)cccc1S(=O)(=O)Nc1noc2cc(Cn3cccn3)c3c(c12)OCC3]

The title compound was synthesized according to procedures analogous to Example 1, Steps 6-7. LC-MS calc. for C₂₇H₃₁N₆O₅S [M+H]⁺: m/z=551.2; Found 551.1.

### Step 4. N-(4-((1H-Pyrazol-1-yl)methyl)-2,3-dihydrobenzofuro[7,6-d]isoxazol-8-yl)-3-(7-((1-(2-(2,6-dioxopiperidin-3-yl)-1,3-dioxoisoindolin-5-yl)piperidin-4-yl)methyl)-2,7-diazaspiro[3.5]nonan-2-yl)-2-methoxybenzenesulfonamide

The title compound was synthesized according to procedures analogous to Example 1, Step 8, using 1-(2-(2,6-dioxopiperidin-3-yl)-1,3-dioxoisoindolin-5-yl)piperidine-4-carbaldehyde (prepared as described by WO2023/245150 A1). LC-MS calc. for C₄₆H₅₀N₉O₉S [M+H]⁺: m/z=904.3; Found 904.8.

### Example 13. N-(4-((1H-Pyrazol-1-yl)methyl)-2,3-dihydrobenzofuro[7,6-d]isoxazol-8-yl)-3-(7-(((S)-1-(2-((S)-2,6-dioxopiperidin-3-yl)-1-oxoisoindolin-5-yl)pyrrolidin-3-yl)methyl)-2,7-diazaspiro[3.5]nonan-2-yl)-2-methoxybenzenesulfonamide

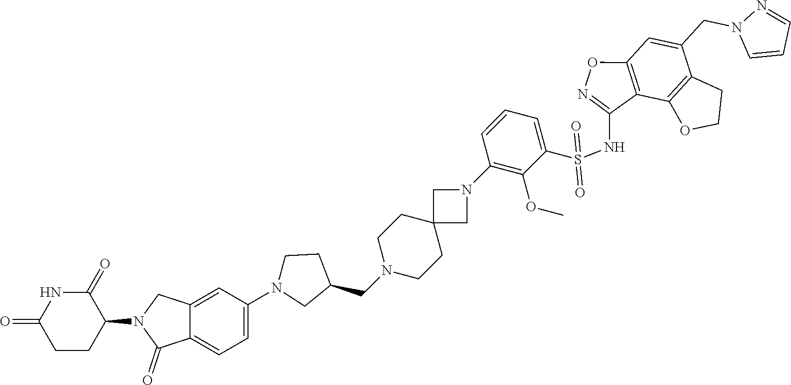 [SMILES: COc1c(N2CC3(CCN(C[C@@H]4CCN(c5ccc6c(c5)CN([C@H]5CCC(=O)NC5=O)C6=O)C4)CC3)C2)cccc1S(=O)(=O)Nc1noc2cc(Cn3cccn3)c3c(c12)OCC3]

The title compound was synthesized according to procedures analogous to Example 1, Step 8, using N-(4-((1H-pyrazol-1-yl)methyl)-2,3-dihydrobenzofuro[7,6-d]isoxazol-8-yl)-2-methoxy-3-(2,7-diazaspiro[3.5]nonan-2-yl)benzenesulfonamide (from Example 12, Step 3). LC-MS calc. for C₄₅H₅₀N₉O₈S [M+H]⁺: m/z=876.3; Found 875.7.

### Example 14. N-(4-((1H-Pyrazol-1-yl)methyl)-2,3-dihydrobenzofuro[7,6-d]isoxazol-8-yl)-3-(7-((1-(2-(2,6-dioxopiperidin-3-yl)-1,3-dioxoisoindolin-4-yl)piperidin-4-yl)methyl)-2,7-diazaspiro[3.5]nonan-2-yl)-2-methoxybenzenesulfonamide

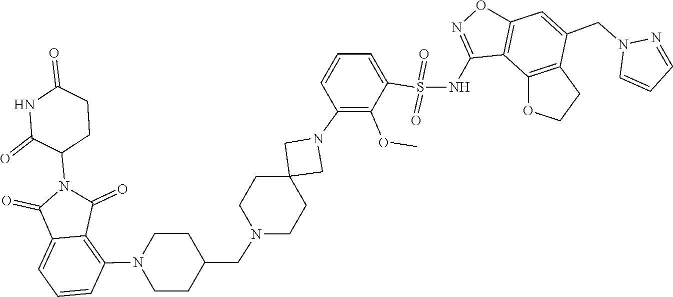 [SMILES: COc1c(N2CC3(CCN(CC4CCN(c5cccc6c5C(=O)N(C5CCC(=O)NC5=O)C6=O)CC4)CC3)C2)cccc1S(=O)(=O)Nc1noc2cc(Cn3cccn3)c3c(c12)OCC3]

The title compound was synthesized according to procedures analogous to Example 6, Step 3, using N-(4-((1H-pyrazol-1-yl)methyl)-2,3-dihydrobenzofuro[7,6-d]isoxazol-8-yl)-2-methoxy-3-(2,7-diazaspiro[3.5]nonan-2-yl)benzenesulfonamide (from Example 12, Step 3) and 2-(2,6-dioxopiperidin-3-yl)-4-(4-(hydroxymethyl)piperidin-1-yl)isoindoline-1,3-dione (prepared as described by WO2023/245150 A1). ¹H NMR (400 MHz, DMSO-d₆) δ 11.03 (s, 1H), 8.93 (s, 1H), 7.77 (d, J=2.3 Hz, 1H), 7.63 (t, J=7.8 Hz, 1H), 7.43 (d, J=1.9 Hz, 1H), 7.28 (d, J=7.6 Hz, 2H), 7.23-6.99 (m, 2H), 6.85-6.68 (m, 1H), 6.64 (s, 1H), 6.24 (t, J=2.1 Hz, 1H), 5.35 (s, 2H), 5.02 (dd, J=12.6, 5.5 Hz, 1H), 4.67 (t, J=8.9 Hz, 2H), 3.64 (d, J=15.1 Hz, 7H), 3.57 (s, 2H), 3.42 (d, J=11.8 Hz, 2H), 3.20-2.72 (m, 9H), 2.65-2.48 (m, 2H), 2.17-1.72 (m, 8H), 1.54-1.31 (m, 2H). LC-MS calc. for C₄₆H₅₀N₉O₉S [M+H]⁺: m/z=904.3; Found 904.5.

### Example 15. N-(4-((1H-Pyrazol-1-yl)methyl)-2,3-dihydrobenzofuro[7,6-d]isoxazol-8-yl)-4-(7-(((S)-1-(2-((S)-2,6-dioxopiperidin-3-yl)-1-oxoisoindolin-5-yl)pyrrolidin-3-yl)methyl)-2,7-diazaspiro[3.5]nonan-2-yl)benzenesulfonamide

The title compound was synthesized according to procedures analogous to Example 1, Steps 6-8, using 4-bromobenzenesulfonyl chloride. LC-MS calc. for C₄₄H₄₈N₉O₇S [M+H]⁺: m/z=846.3; Found 846.7.

### Example A. HiBit KAT6A Degradation Assessment

The degradation activity of compounds was evaluated by measuring levels of KAT6A protein using the HiBiT system. These studies were conducted in HeLa cells with a HiBit tagged KAT6A. Cells were maintained in a 37° C. incubator at 5% CO₂ in EMEM or DMEM (ATCC, 30-2003) supplemented with 10% v/v FBS (Gibco, 26140-079) and 1% penicillin streptomycin. Cells were seeded in 384-well plates at a density of 2,000 cells/well. Compounds dissolved in DMSO were added in 9-point serial dilution. After 18-24 h of treatment, KAT6A levels were measured using the Nano-Glo® HiBiT Lytic Detection System (Promega catalog number N3030) per manufacturer's instructions. Luminescence signal was measured with a multimode plate reader (Envision 2105, Perkin Elmer or PHERAStar FSX, BMG Labtech). Luminescence values were normalized to background and DMSO controls to quantify KAT6A for each condition. Results were analyzed, and DC₅₀ and Dₘₐₓ values obtained using a four-parameter logistic curve fit. The results are summarized in Table 3 (18 h of treatment) and Table 4 (24 h of treatment).

TABLE 3
HiBit KAT6A Degradation

| Ex. | DC₅₀ | Dₘₐₓ |
|---|---|---|
| 1 | A | A |
| 2 | B | A |
| 3 | B | A |
| 4 | B | A |
| 5 | D | C |
| 6 | C | B |
| 7 | C | B |
| 8 | A | A |
| 9 | A | B |

In Table 3, column DC₅₀, an “A” denotes DC₅₀<10 nM; a “B” denotes 10 nM≤DC₅₀<100 nM; a “C” denotes 100 nM≤DC₅₀<1000 nM; D represents that a DC₅₀ was not calculated due to screening parameters. In Table 3, column Dₘₐₓ, an “A” denotes Dₘₐₓ>75%; a “B” denotes 50%<Dₘₐₓ≤75%; a “C” denotes 25%<Dₘₐₓ≤50%; D represents that a Dₘₐₓ≤25%.

TABLE 4
HiBiT KAT6A Degradation

| Ex. | DC₅₀ | Dₘₐₓ |
|---|---|---|
| 1 | B | B |
| 10 | A | A |
| 11 | A | B |
| 12 | B | A |
| 13 | A | A |
| 14 | A | A |
| 15 | B | B |

In Table 4, column DC₅₀, an “A” denotes DC₅₀<10 nM; a “B” denotes 10 nM<DC₅₀<100 nM; a “C” denotes 100 nM≤DC₅₀<1000 nM; D represents that a DC₅₀ was not calculated due to screening parameters. In Table 4, column Dₘₐₓ, an “A” denotes Dₘₐₓ>75%; a “B” denotes 50%<Dₘₐₓ≤75%; a “C” denotes 25%<Dₘₐₓ≤50%; D represents that a Dₘₐₓ≤25%.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of Formula (I):
ULM-Y PTM (I)
or a pharmaceutically acceptable salt or solvate thereof,
wherein PTM is a moiety of Formula IA: 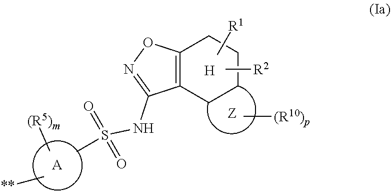
wherein
ring H is a 6-membered aromatic ring or a 6-membered heteroaromatic ring;
ring Z is a 5- to 7-membered cycloalkenyl ring, a 5- to 7-membered heterocycloalkenyl ring, or 5- to 7-membered heteroaromatic ring;
Y is a covalent bond, or chemical moiety that links PTM to ULM;
** is a point of attachment to Y;
Ring A is a C₆-C₁₀ aryl ring or a 5-10 membered heteroaryl ring;
R¹ is H or 5-6 membered heteroaryl ring optionally substituted by C₁-C₃ alkyl;
R² is H or —(C(R⁸)₂)ₙ-(5-9 membered heteroaryl ring) optionally substituted by halogen, C₁-C₃ alkyl, —CH₂OH, or —OH;
each R⁵ is independently D, halogen, oxo, —OH, —CN, —NO₂, —C₁-C₆alkyl, —C₂-C₆alkenyl, —C₁-C₆alkynyl, haloalkyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{d})(OR^{c}) or —S(O)₂R^{b};
m is 0, 1, 2, 3, or 4;
each R⁸ is independently H, halogen, or C₁-C₄ alkyl;
n is 0 or 1;
each R¹⁰ is independently H, halogen, C₁₋₄alk-O—C₁₋₄alkyl, C₁-C₄ alkoxy or C₁-C₄ alkyl;
p is 1, 2, 3, 4, 5, 6, 7 or 8;
each R^{a} is independently H, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)R^{c}R^{d}, —P(O)OR^{c}OR^{b}, —S(O)R^{b}, —S(O)NR^{c}R^{d}, —S(O)₂R^{b}, —S(O)₂NR^{c}R^{d}, SiR^{b}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl;
each R^{b}, is independently H, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl;
each R^{c} or R^{d} is independently H, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; or
R^{c} and R^{d}, together with the atom to which they are both attached, form a monocyclic or multicyclic heterocycloalkyl, or a monocyclic or multicyclic heterocycloalkenyl group; and
ULM is a small molecule E3 Ubiquitin Ligase binding moiety that binds a Cereblon E3 Ubiquitin Ligase.

2. The compound according to claim 1, wherein ring Z is a or a 5- to 7-membered cycloalkenyl ring.

3. The compound according to claim 2, wherein ring Z is a cyclopentenyl ring or a cyclohexenyl ring.

4. The compound according to claim 1, wherein ring Z is or a 5- to 7-membered heterocycloalkenyl ring.

5. The compound according to claim 4, wherein ring Z is a dihydrofuran ring, a dihydro-2H-pyran ring, a dihydro-dioxepine ring, a dioxole ring, or a dihydrothiophene ring.

6. The compound according to claim 1, wherein ring Z is a 5- to 7-membered heteroaromatic ring.

7. The compound according to claim 6, wherein ring Z is a furan ring or an imidazole ring.

8. A compound of claim 1, or a pharmaceutically acceptable salt or solvate thereof, wherein PTM is a moiety of Formula IIa: 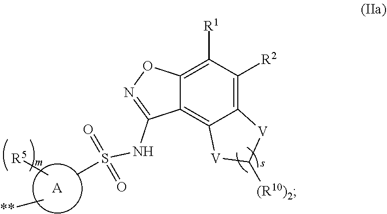
wherein
each V is independently O, S, NR¹⁰ or C(R¹⁰)₂; and
s is 1, 2, 3 or 4.

9. The compound according to claim 1, wherein Y is a chemical moiety that links PTM to ULM.

10. The compound according to claim 1, wherein R¹ is H.

11. The compound according to claim 1, wherein R² is —(C(R⁸)₂)ₙ-(5-6 membered heteroaryl) optionally substituted by halogen, C₁-C₃ alkyl, —CH₂OH, or —OH.

12. The compound according to claim 11, wherein the 5-6 membered heteroaryl is a pyrazole, a pyrrole, a pyridine or a pyridazine.

13. The compound according to claim 11, wherein the 5-6 membered heteroaryl is a pyrazole.

14. The compound according to claim 1, wherein n is 0.

15. The compound according to claim 1, wherein n is 1.

16. The compound according to claim 1, wherein ring A is C₆-C₁₀ aryl.

17. The compound according to claim 16, wherein ring A is phenyl.

18. The compound according to claim 1, wherein each R⁵ is C₁-C₄ alkoxy.

19. The compound according to claim 1, wherein at least one R⁵ is C₁-C₄ alkoxy.

20. The compound according to claim 1, wherein m is 0.

21. The compound according to claim 1, wherein m is 1.

22. The compound according to claim 1, wherein m is 2.

23. The compound according to claim 1, wherein each R⁸ is H.

24. The compound according to claim 1, wherein Y is represented by the formula:
-(G)_{q}-,
wherein:
q is an integer from 1 to 14;
each G is independently selected from the group consisting of CR^{1a}R^{1b}, O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}=CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{1c}(CR^{1a}R^{1b})₁₋₄, NR^{1c}C(═NCN)NR^{1d} NR^{1c}C(═NCN), NR^{1c}C(═CNO₂)NR^{1d}, 3-15 membered cycloalkyl, optionally substituted with 1-6 R^{1a} or R^{1b} groups, 3-15 membered heterocyclyl optionally substituted with 1-6 R^{1a} or R^{1b} groups, aryl optionally substituted with 1-6 R^{1a} or R^{1b} groups, or heteroaryl optionally substituted with 1-6 R^{1a} or R^{1b} groups,
wherein R^{1a}, R^{1b}, R^{1c}, R^{1d} and R^{1c} are each independently, —H, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂.

25. The compound according to claim 24 wherein q is an integer from 1 to 5.

26. The compound according to claim 1, wherein Y is 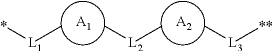
wherein
* is a point of attachment to Ring A of the PTM;
** is a point of attachment to ULM;
L₁, L₂, and L₃ are each independently a bond, CR^{1a}R^{1b}, O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d} NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{1c}(CR^{1a}R^{1b})₁₋₄, NR^{1c}C(═NCN)NR^{1d} NR^{1c}C(═NCN), NR^{1c}C(═CNO₂)NR^{1d};
ring A₁ and ring A₂ are each independently 3-15 membered cycloalkyl, optionally substituted with 1-6 R^{1a} or R^{1b} groups, 3-15 membered heterocyclyl optionally substituted with 1-6 R^{1a} or R^{1b} groups, aryl optionally substituted with 1-6 R^{1a} or R^{1b} groups, or heteroaryl optionally substituted with 1-6 R^{1a} or R^{1b} groups,
wherein R^{1a}, R^{1b}, R^{1c}, R^{1d} and R^{1e} are each independently, —H, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂.

27. The compound according to claim 1, wherein Y is 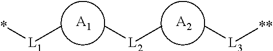
wherein
* is a point of attachment to ring A of the PTM;
L₁ is a bond, (C(R¹⁰)₂)ₚ, or CO;
L₂ is a bond, (C(R¹⁰)₂)ₚ, or CO;
L₃ is a bond, (C(R¹⁰)₂)ₚ, or CO;
p is 1, 2, 3 or 4;
each R¹⁰ is independently H or C₁-C₄ alkyl;
ring A₁ is a 3-7 membered cycloalkyl group, a 4-10-membered heterocycloalkyl group, an aryl group, or a heteroaryl group; and
ring A₂ is a 3-15 membered cycloalkyl group, a 4-15 membered heterocycloalkyl group, an aryl group, or a heteroaryl group.

28. The compound according to claim 1, wherein Y is 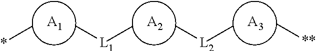
wherein
** is a point of attachment to Ring A of the PTM;
* is a point of attachment to ULM;
L₁ and L₂ are each independently a bond, CR^{1a}R^{1b} O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{1c}(CR^{1a}R^{1b})₁₋₄, NR^{1c}C(═NCN)NR^{1d} NR^{1c}C(═NCN), NR^{1c}C(═CNO₂)NR^{1d};
ring A₁, ring A₂ and ring A₃ are each independently 3-15 membered cycloalkyl, optionally substituted with 1-6 R^{1a} or R^{1b} groups, 3-15 membered heterocyclyl optionally substituted with 1-6 R^{1a} or R^{1b} groups, aryl optionally substituted with 1-6 R^{1a} or R^{1b} groups, or heteroaryl optionally substituted with 1-6 R^{1a} or R^{1b} groups,
wherein R^{1a}, R^{1b}, R^{1c}, R^{1d} and R^{1e} are each independently, —H, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —CF₃, —CHF₂, —CH₂F, —N₀₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂.

29. The compound according to claim 1, wherein Y is 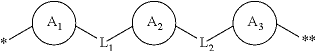
wherein
** is a point of attachment to Ring A of the PTM;
* is a point of attachment to ULM;
L₁ is a bond, (C(R¹⁰)₂)ₚ, or CO;
L₂ is a bond, (C(R¹⁰)₂)ₚ, or CO;
p is 1, 2, 3 or 4;
each R¹⁰ is independently H or C₁-C₄ alkyl;
ring A₁ is a 3-7 membered cycloalkyl group, a 4-10-membered heterocycloalkyl group, an aryl group, or a heteroaryl group; and
ring A₂ is a 3-15 membered cycloalkyl group, a 4-15 membered heterocycloalkyl group, an aryl group, or a heteroaryl group; and
ring A₃ is a 3-15 membered cycloalkyl group, a 4-15 membered heterocycloalkyl group,
an aryl group, or a heteroaryl group.

30. The compound according to claim 26, wherein L₃ is a bond.

31. The compound according to claim 26, wherein L₁ is a bond.

32. The compound according to claim 26, wherein L₂ is (C(R¹⁰)₂)ₚ.

33. The compound according to claim 26, wherein ring A₁ is a 4-10-membered heterocycloalkyl group.

34. The compound according to claim 33, wherein ring A₁ is a piperazine group, a morpholine group, a piperidine group, a pyrrolidine group, an azetidine group or an azabicyclo-alkyl group.

35. The compound according to claim 33, wherein ring A₁ is a piperidine group or pyrrolidine group.

36. The compound according to claim 26, wherein ring A₂ is a 4-15-membered heterocycloalkyl group.

37. The compound according to claim 36, wherein ring A₂ is a piperazine group, a morpholine group, a piperidine group, a pyrrolidine group, an azetidine group, a diazaspiroalkyl group or an azabicycloalkyl group.

38. The compound according to claim 36, wherein ring A₂ is a piperazine group or a diazaspirononane group.

39. The compound according to claim 26, wherein Y is 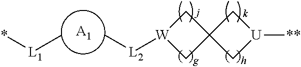
wherein
W is C(R¹⁵) or N;
U is C(R¹⁵) or N;
R¹⁵ is H, F, C₁₋₄alkyl or C₁₋₄alkoxide;
each g and h is independently 1, 2 or 3; and
each j and k is independently 0, 1, 2 or 3.

40. The compound according to claim 39, wherein j and g are each 2.

41. The compound according to claim 39, wherein k and h are each 1.

42. The compound according to claim 39, wherein W is N.

43. The compound according to claim 39, wherein U is N.

44. The compound according to claim 1, wherein ULM is 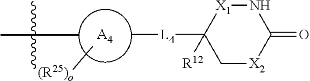
wherein: [symbol]
is a point of attachment to Y or PTM;
Ring A₄ is a monocyclic, bicyclic or tricyclic aryl, heteroaryl or heterocycle group,
L₄ is a bond, —O—, —S—, —NR^{a}—, —C(R^{a})₂— —C(O)NR^{a}—;
X₁ is CH₂, CO, CH═CH (when X₂═CO), or N═CH (when X₂═CO);
X₂ is CH₂, CO, CH═CH (when X₁═CO), or N═CH (when X₁═CO);
R¹² is H, optionally substituted C₁₋₄ alkyl, C₁₋₄ alkoxyl, C₁₋₄haloalkyl, —CN, —OR^{a}, —OR^{b} or —SR^{b};
each R²⁵ is independently H, halogen, oxo, —OH, —CN, —NO₂, —C₁-C₆alkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{d})(OR^{c}) or —S(O)₂R^{b};
each R^{a} is independently H, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)R^{c}R^{d}, —P(O)OR^{c}OR^{b}, —S(O)R^{b}, —S(O)NR^{c}R^{d}, —S(O)₂R^{b}, —S(O)₂NR^{c}R^{d}, SiR^{b}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl;
each R^{b}, is independently H, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl;
each R^{c} or R^{d} is independently H, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; or
R^{c} and R^{d}, together with the atom to which they are both attached, form a monocyclic or multicyclic heterocycloalkyl, or a monocyclic or multicyclic heterocyclo-alkenyl group; and
o is 1, 2, 3, 4, or 5.

45. The compound according to claim 44, wherein Ring A₄ is a bicyclic heterocycle group.

46. The compound according to claim 44, wherein L₄ is a bond.

47. The compound according to claim 44, wherein R¹² is H.

48. The compound according to claim 44, wherein R²⁵ is H.

49. The compound according to claim 44, wherein X₁ is CO.

50. The compound according to claim 44, wherein X₂ is CH₂.

51. The compound according claim 44, wherein ULM is 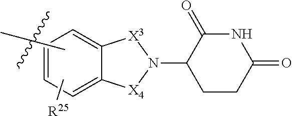
wherein: [symbol]
is a point of attachment to Y or PTM;
X₃ is CH₂, CO, CH═CH (when X₄═CO), or N═CH (when X₄═CO); and
X₄ is CH₂, CO, CH═CH (when X₃═CO), or N═CH (when X₃═CO).

52. The compound according claim 51, wherein X₃ is CH₂.

53. The compound according claim 51, wherein X₃ is CO.

54. The compound according claim 51, wherein X₄ is CH₂.

55. The compound according claim 51, wherein X₄ is CO.

56. The compound according to claim 1, wherein Y-PTM is a compound of Formula IIIa or Formula IIIb: 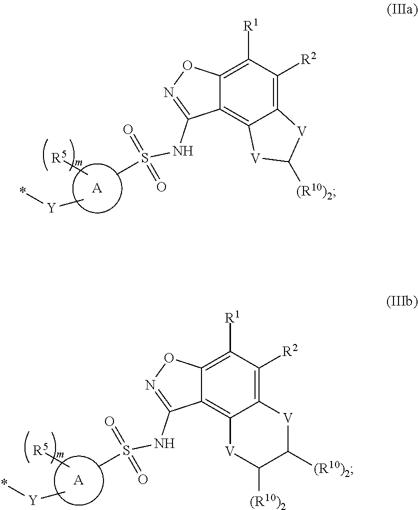
or a pharmaceutically acceptable salt thereof; wherein * is a point of attachment to ULM.

57. The compound according to claim 1, wherein Y-PTM is a compound of Formula IVa or Formula IVb: 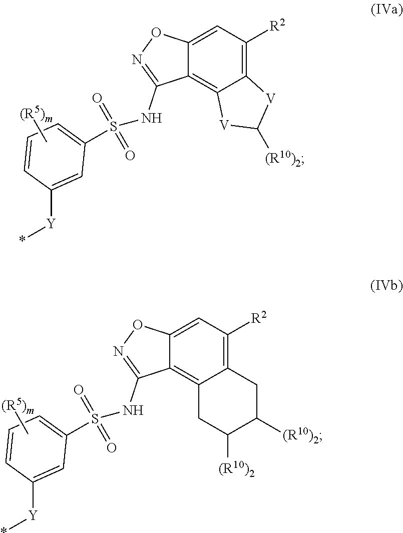
or a pharmaceutically acceptable salt thereof; wherein * is a point of attachment to ULM.

58. The compound according to claim 1, wherein Y-PTM is a compound of Formula Va or Formula Vb: 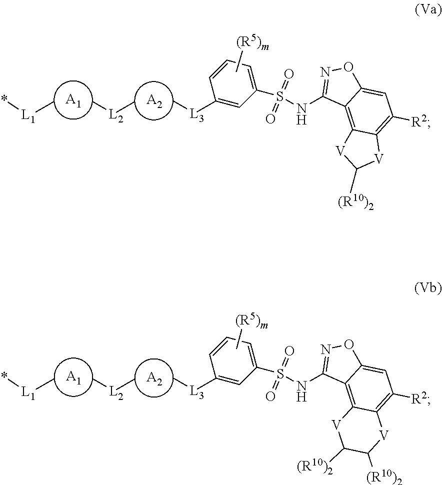
or a pharmaceutically acceptable salt thereof; wherein
* is a point of attachment to ULM;
L₁ is a bond, (C(R¹⁰)₂)ₚ, or CO;
L₂ is a bond, (C(R¹⁰)₂)ₚ, or CO;
L₃ is a bond, (C(R¹⁰)₂)ₚ, or CO;
p is 1, 2, 3 or 4;
each R¹⁰ is independently H or C₁-C₄ alkyl;
ring A₁ is a 3-15 membered cycloalkyl group, a 4-15-membered heterocycloalkyl group, an aryl group, or a heteroaryl group; and
ring A₂ is a 3-15 membered cycloalkyl group, a 4-15-membered heterocycloalkyl group, an aryl group, or a heteroaryl group.

59. The compound according to claim 58, wherein ring A₂ is

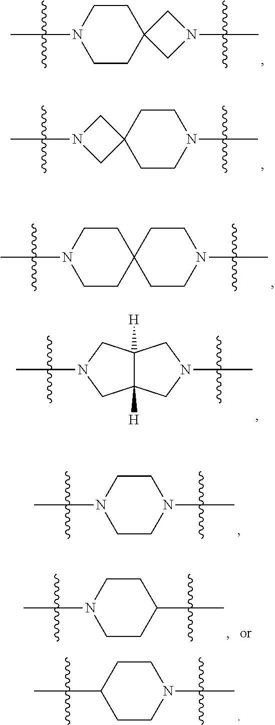

60. The compound of claim 58, wherein the compound is a compound of Formula Va-1: 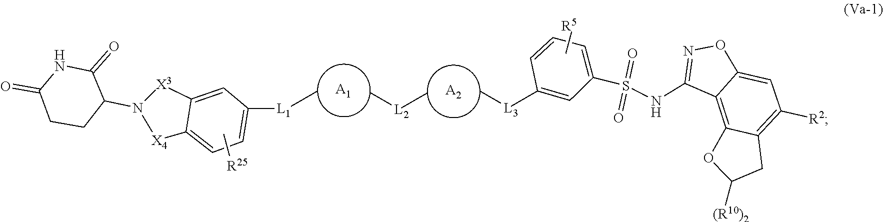
wherein
ring A₁ and ring A₂ are each independently selected from: 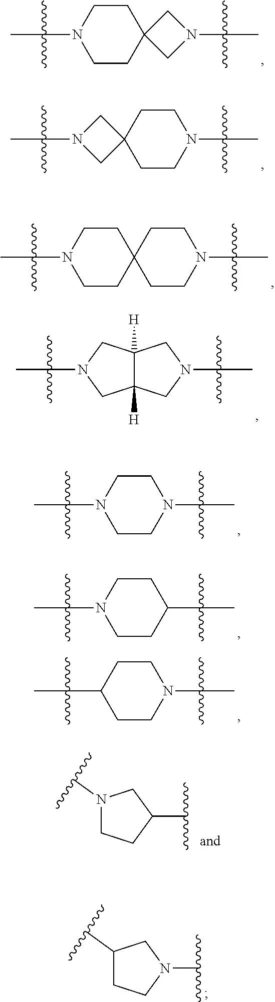
L₂ is a bond or (CH₂)ₚ;
L₃ is a bond or (CH₂)ₚ;
p is 1, 2, 3 or 4;
each R¹⁰ is independently H or C₁-C₄ alkyl;
R² is —(CH₂)ₙ-(5-9 membered heteroaryl ring);
n=0 or 1;
X₃ is CH₂, CO, CH═CH (when X₄═CO), or N═CH (when X₄═CO); and
X₄ is CH₂, CO, CH═CH (when X₃═CO), or N═CH (when X₃═CO).

61. The compound according to claim 1, wherein Y-PTM is a compound of Formula VIa or Formula VIb: 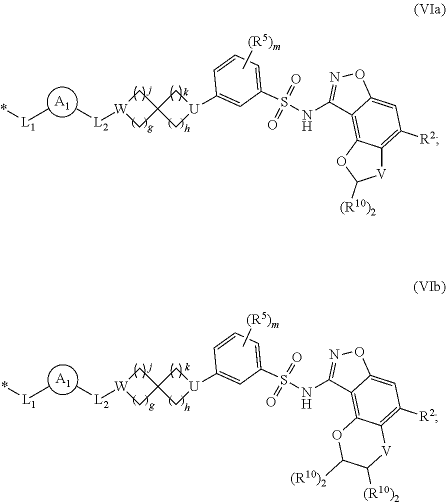
or a pharmaceutically acceptable salt thereof; wherein
* is a point of attachment to ULM;
L₁ is a bond, (C(R¹⁰)₂)ₚ, or CO;
L₂ is a bond, (C(R¹⁰)₂)ₚ, or CO;
L₃ is a bond, (C(R¹⁰)₂)ₚ, or CO;
p is 1, 2, 3 or 4;
each R¹⁰ is independently H or C₁-C₄ alkyl;
ring A₁ is a 3-15 membered cycloalkyl group, a 4-15-membered heterocycloalkyl group, an aryl group, or a heteroaryl group;
W is C(R¹⁵) or N;
U is C(R¹⁵) or N;
V is O or C(R¹⁰)₂;
R¹⁵ is H, fluoro, C₁₋₄alkyl or C₁₋₄alkoxide;
each g and h is independently 1, 2 or 3; and
each j and k is independently 0, 1, 2 or 3.

62. The compound according to claim 1, wherein each V is O.

63. The compound according to claim 1, wherein PTM-Y-ULM is a compound of Formula VIIa or Formula VIIb: 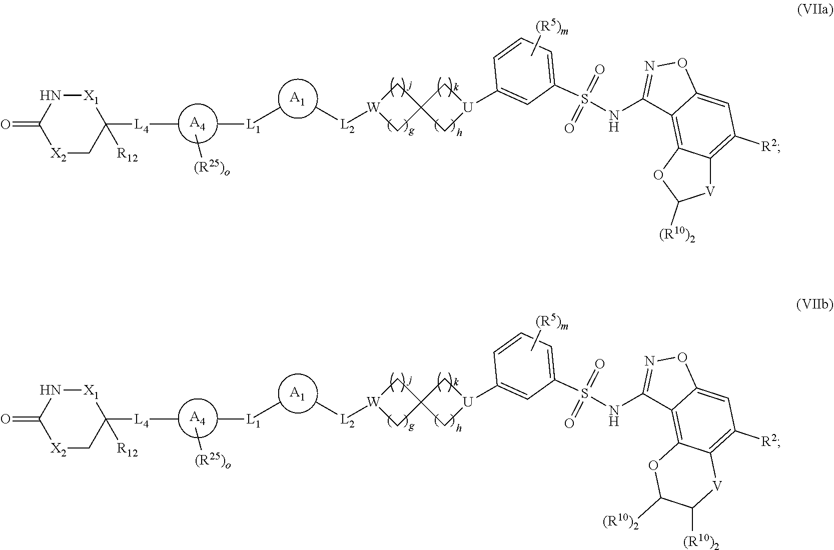
or a pharmaceutically acceptable salt thereof; wherein
L₁ is a bond, (C(R¹⁰)₂)ₚ, or CO;
L₂ is a bond, (C(R¹⁰)₂)ₚ, or CO;
p is 1, 2, 3 or 4;
each R¹⁰ is independently H or C₁-C₄ alkyl;
W is C(R¹⁵) or N;
U is C(R¹⁵) or N;
V is O or C(R¹⁰)₂;
R¹⁵ is H, fluoro, C₁₋₄alkyl or C₁₋₄alkoxide;
each g and h is independently 1, 2 or 3;
each j and k is independently 0, 1, 2 or 3;
ring A₁ is a 3-7 membered cycloalkyl group, a 4-10-membered heterocycloalkyl group, an aryl group, or a heteroaryl group;
Ring A₄ is a monocyclic, bicyclic or tricyclic aryl, heteroaryl or heterocycle group;
L₄ is a bond, —O—, —S—, —NR^{a}—, —C(R^{a})₂— —C(O)NR^{a}—;
X₁ is CH₂, CO, CH═CH (when X₂═CO), or N═CH (when X₂═CO);
X₂ is CH₂, CO, CH═CH (when X₁═CO), or N═CH (when X₁═CO);
R¹² is H, D, optionally substituted C₁₋₄ alkyl, C₁₋₄ alkoxyl, C₁₋₄haloalkyl, —CN, —OR^{a}, —OR^{b} or —SR^{b};
each R²⁵ is independently H, halogen, oxo, —OH, —CN, —NO₂, —C₁-C₆alkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{d})(OR^{c}) or —S(O)₂R^{b};
each R^{a} is independently H, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)R^{c}R^{b}, —P(O)OR^{c}OR^{b}, —S(O)R^{b}, —S(O)NR^{c}R^{d}, —S(O)₂R^{b}, —S(O)₂NR^{c}R^{d}, SiR^{b}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl;
each R^{b}, is independently H, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl;
each R^{c} or R^{d} is independently H, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; or
R^{c} and R^{d}, together with the atom to which they are both attached, form a monocyclic or multicyclic heterocycloalkyl, or a monocyclic or multicyclic heterocyclo-alkenyl group; and
o is 1, 2, 3, 4, or 5.

64. The compound according to claim 63, wherein PTM-Y-ULM is a compound of Formula VIIIa or Formula VIIIb: 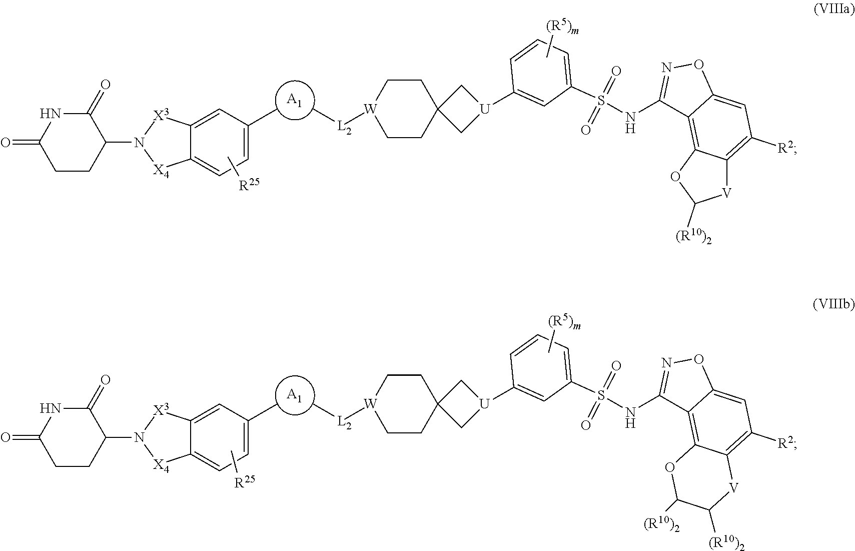
or a pharmaceutically acceptable salt thereof; wherein
X₃ is CH₂, CO, CH═CH (when X₄═CO), or N═CH (when X₄═CO); and
X₄ is CH₂, CO, CH═CH (when X₃═CO), or N═CH (when X₃═CO).

65. The compound according to claim 63, wherein PTM-Y-ULM is a compound of Formula IXa or Formula IXb: 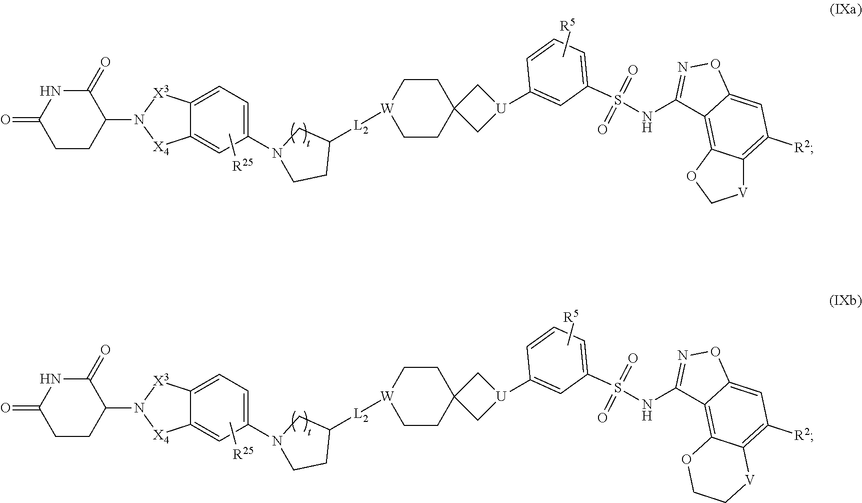
or a pharmaceutically acceptable salt thereof; wherein
t is 1 or 2.

66. The compound according to claim 58, wherein L₂ is a methylene group.

67. The compound according to claim 58, wherein PTM-Y-ULM is a compound of Formula Xa: 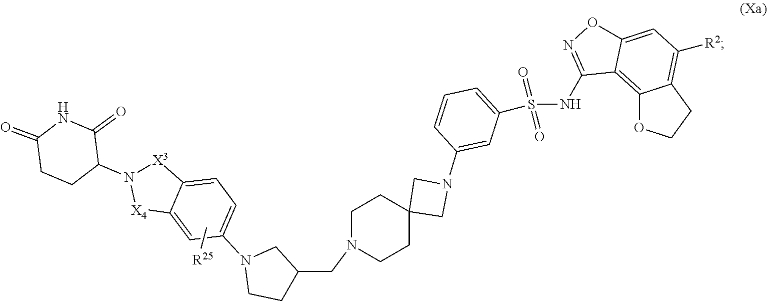
or a pharmaceutically acceptable salt thereof.

68. The compound according to claim 1, wherein each R⁵ is independently halogen, cyano, C₁-C₄ alkyl, haloalkyl, cyclopropyl, C₁-C₄ alkoxy, haloalkoxy, —O-cyclopropyl, —CH₂—O—CH₃, —C(O)OCH₃, or —C(O)N(H)CH₃.

69. The compound according to claim 1 that is:
N-(4-((1H-Pyrazol-1-yl)methyl)-2,3-dihydrobenzofuro[7,6-d]isoxazol-8-yl)-3-(7-(((S)-1-(2-((S)-2,6-dioxopiperidin-3-yl)-1-oxoisoindolin-5-yl)pyrrolidin-3-yl)methyl)-2,7-diazaspiro [3.5]nonan-2-yl)benzenesulfonamide;
N-(4-((1H-Pyrazol-1-yl)methyl)-2,3-dihydrobenzofuro[7,6-d]isoxazol-8-yl)-3-((3aS,6aS)-5-(((S)-1-(2-((S)-2,6-dioxopiperidin-3-yl)-1-oxoisoindolin-5-yl)pyrrolidin-3-yl)methyl)hexahydropyrrolo[3,4-c]pyrrol-2(1H)-yl)benzenesulfonamide;
N-(4-((1H-Pyrazol-1-yl)methyl)-2,3-dihydrobenzofuro[7,6-d]isoxazol-8-yl)-3-(9-(((S)-1-(2-((S)-2,6-dioxopiperidin-3-yl)-1-oxoisoindolin-5-yl)pyrrolidin-3-yl)methyl)-3, 9-diazaspiro [5.5]undecan-3-yl)benzenesulfonamide;
N-(4-((1H-Pyrazol-1-yl)methyl)-2,3-dihydrobenzofuro[7,6-d]isoxazol-8-yl)-3-(2-(((S)-1-(2-((S)-2,6-dioxopiperidin-3-yl)-1-oxoisoindolin-5-yl)pyrrolidin-3-yl)methyl)-2,7-diazaspiro [3.5]nonan-7-yl)benzenesulfonamide;
N-(4-((1H-Pyrazol-1-yl)methyl)-2,3-dihydrobenzofuro[7,6-d]isoxazol-8-yl)-3-(4-((((S)-1-(2-((S)-2,6-dioxopiperidin-3-yl)-1-oxoisoindolin-5-yl)pyrrolidin-3-yl)methyl)amino) piperidin-1-yl)benzenesulfonamide;
N-(4-((1H-Pyrazol-1-yl)methyl)-2,3-dihydrobenzofuro[7,6-d]isoxazol-8-yl)-3-(4-(7-(2-(2,6-dioxopiperidin-3-yl)-1,3-dioxoisoindolin-5-yl)-7-azaspiro[3.5]nonan-2-yl)piperazin-1-yl)benzenesulfonamide;
N-(4-((1H-Pyrazol-1-yl)methyl)-2,3-dihydrobenzofuro[7,6-d]isoxazol-8-yl)-3-(4-((1-(2-(2,6-dioxopiperidin-3-yl)-1,3-dioxoisoindolin-4-yl)piperidin-4-yl)methyl)piperazin-1-yl)benzenesulfonamide;
N-(4-((1H-Pyrazol-1-yl)methyl)-2,3-dihydrobenzofuro[7,6-d]isoxazol-8-yl)-3-(4-((1-((1-(2-(2,6-dioxopiperidin-3-yl)-1,3-dioxoisoindolin-5-yl)pyrrolidin-3-yl)methyl)piperidin-4-yl)methyl)piperazin-1-yl)benzenesulfonamide;
N-(4-((1H-Pyrazol-1-yl)methyl)-2,3-dihydrobenzofuro[7,6-d]isoxazol-8-yl)-3-(4-((1-((1-(2-(2,6-dioxopiperidin-3-yl)-1,3-dioxoisoindolin-5-yl)piperidin-4-yl)methyl)piperidin-4-yl)methyl)piperazin-1-yl)benzenesulfonamide;
or a pharmaceutically acceptable salt thereof.

70. The compound according to claim 1 that is:
N-(4-((1H-Pyrazol-1-yl)methyl)-2,3-dihydrobenzofuro[7,6-d]isoxazol-8-yl)-3-(7-(((S)-1-(2-((S)-2,6-dioxopiperidin-3-yl)-1-oxoisoindolin-5-yl)pyrrolidin-3-yl)methyl)-2,7-diazaspiro[3.5]nonan-2-yl)-2,6-dimethoxybenzenesulfonamide;
N-(4-((1H-Pyrazol-1-yl)methyl)-2,3-dihydrobenzofuro[7,6-d]isoxazol-8-yl)-3-(7-((1-(2-(2,6-dioxopiperidin-3-yl)-1,3-dioxoisoindolin-5-yl)piperidin-4-yl)methyl)-2,7-diazaspiro[3.5]nonan-2-yl)-2,6-dimethoxybenzenesulfonamide;
N-(4-((1H-Pyrazol-1-yl)methyl)-2,3-dihydrobenzofuro[7,6-d]isoxazol-8-yl)-3-(7-((1-(2-(2,6-dioxopiperidin-3-yl)-1,3-dioxoisoindolin-5-yl)piperidin-4-yl)methyl)-2,7-diazaspiro[3.5]nonan-2-yl)-2-methoxybenzenesulfonamide;
N-(4-((1H-Pyrazol-1-yl)methyl)-2,3-dihydrobenzofuro[7,6-d]isoxazol-8-yl)-3-(7-(((S)-1-(2-((S)-2,6-dioxopiperidin-3-yl)-1-oxoisoindolin-5-yl)pyrrolidin-3-yl)methyl)-2,7-diazaspiro[3.5]nonan-2-yl)-2-methoxybenzenesulfonamide;
N-(4-((1H-Pyrazol-1-yl)methyl)-2,3-dihydrobenzofuro[7,6-d]isoxazol-8-yl)-3-(7-((1-(2-(2,6-dioxopiperidin-3-yl)-1,3-dioxoisoindolin-4-yl)piperidin-4-yl)methyl)-2,7-diazaspiro[3.5]nonan-2-yl)-2-methoxybenzenesulfonamide;
N-(4-((1H-Pyrazol-1-yl)methyl)-2,3-dihydrobenzofuro[7,6-d]isoxazol-8-yl)-4-(7-(((S)-1-(2-((S)-2,6-dioxopiperidin-3-yl)-1-oxoisoindolin-5-yl)pyrrolidin-3-yl)methyl)-2,7-diazaspiro[3.5]nonan-2-yl)benzenesulfonamide;
or a pharmaceutically acceptable salt thereof.

71. The compound according to claim 1, in the form of a pharmaceutically acceptable salt.

72. A pharmaceutical composition comprising a compound according to claim 1, or a pharmaceutically acceptable salt thereof, and a pharmaceutically acceptable excipient.

73. A method of treating cancer or solid tumors in a subject in need thereof comprising administering to the subject a compound of claim 1 or a pharmaceutical composition comprising the compound.

74. The method of claim 73, wherein the cancer is selected from the group consisting of lung cancer, mesothelioma, bone cancer, pancreatic cancer, skin cancer, cancer of the head or neck, cutaneous or intraocular melanoma, uterine cancer, ovarian cancer, rectal cancer, cancer of the anal region, stomach cancer, hepatic carcinoma, colon cancer, breast cancer, uterine cancer, carcinoma of the fallopian tubes, carcinoma of the endometrium, carcinoma of the cervix, carcinoma of the vagina, carcinoma of the vulva, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, hematology malignancy, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, glioblastoma, brain stem glioma, pituitary adenoma, and a combination of two or more of the foregoing cancers.

75. The method of claim 73, wherein the solid tumors are selected from the group consisting of breast, lung, colon, brain, prostate, stomach, pancreatic, ovarian, melanoma, endocrine, uterine, testicular, hematologic and bladder.

76. The method of claim 75, wherein the solid tumors are selected from the group consisting of breast, lung, prostate, pancreatic, hematologic and ovarian.

77. The method of claim 73, wherein the cancer is breast cancer.

78. The method of claim 77, wherein the breast cancer is ER+ breast cancer.

79. The method of claim 77, wherein the breast cancer is ER+ HER2−breast cancer.

80. The method of claim 77, wherein the breast cancer is locally advanced or metastatic ER-+ HER2−breast cancer.

81. The method of claim 73, wherein the cancer is lung cancer.

82. The method of claim 81, wherein the lung cancer is non-small cell lung cancer.

83. The method of claim 81, wherein the lung cancer is locally advanced or metastatic non-small cell lung cancer.

84. The method of claim 73, wherein the cancer is prostate cancer.

85. The method of claim 84, wherein the prostate cancer is castration resistant prostate cancer.

86. The method of claim 84, wherein the prostate cancer is locally advanced or metastatic castration resistant prostate cancer.

87. A method of degrading a KAT6 protein comprising contacting the KAT6 protein with a compound of claim 1 or a pharmaceutical composition comprising the compound.
